SEQUENCE LISTING (67 sequences)

SEQ ID NO: 1 - protein, 4 aa, synthetic construct
PSAP

SEQ ID NO: 2 - skipped

SEQ ID NO: 3 - protein, 238 aa, synthetic construct
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 4 - protein, 922 aa, synthetic construct
MATASPRSDTSNNHSGRLQLQVTVSSAKLKRKKNWFGTAIYTEVVVDGEITKTAKSSSSSNPKWDEQLTVNVTPQTTLEFQVWSHRTLKADALLGKATIDLKQALLIHNRKLERVKEQLKLSLENKNGIAQTGELTVVLDGLVIEQENITNCSSSPTIEIQENGDALHENGEPSARTTARLAVEGTNGIDNHVPTSTLVQNSCCSYVVNGDNTPSSPSQVAARPKNTPAPKPLASEPADDTVNGESSSFAPTDNASVTGTPVVSEENALSPNCTSTTVEDPPVQEILTSSENNECIPSTSAELESEARSILEPDTSNSRSSSAFEAAKSRQPDGCMDPVRQQSGNANTETLPSGWEQRKDPHGRTYYVDHNTRTTTWERPQPLPPGWERRVDDRRRVYYVDHNTRTTTWQRPTMESVRNFEQWQSQRNQLQGAMQQFNQRYLYSASMLAAENDPYGPLPPGWEKRVDSTDRVYFVNHNTKTTQWEDPRTQGLQNEEPLPEGWEIRYTREGVRYFVDHNTRTTTFKDPRNGKSSVTKGGPQIAYERGFRWKLAHFRYLCQSNALPSHVKINVSRQTLFEDSFQQIMALKPYDLRRRLYVIFRGEEGLDYGGLAREWFFLLSHEVLNPMYCLFEYAGKNNYCLQINPASTINPDHLSYFCFIGRFIAMALFHGKFIDTGFSLPFYKRMLSKKLTIKDLESIDTEFYNSLIWIRDNNIEECGLEMYFSVDMEILGKVTSHDLKLGGSNILVTEENKDEYIGLMTEWRFSRGVQEQTKAFLDGFNEVVPLQWLQYFDEKELEVMLCGMQEVDLADWQRNTVYRHYTRNSKQIIWFWQFVKETDNEVRMRLLQFVTGTCRLPLGGFAELMGSNGPQKFCIEKVGKDTWLPRSHTCFNRLDLPPYKSYEQLKEKLLFAIEETEGFGQE

SEQ ID NO: 5 - protein, 34 aa, synthetic construct
ETLPSGWEQRKDPHGRTYYVDHNTRTTTWERPQP

SEQ ID NO: 6 - protein, 34 aa, synthetic construct
QPLPPGWERRVDDRRRVYYVDHNTRTTTWQRPTM

SEQ ID NO: 7 - protein, 39 aa, synthetic construct
ENDPYGPLPPGWEKRVDSTDRVYFVNHNTKTTQWEDPRT

SEQ ID NO: 8 - protein, 34 aa, synthetic construct
EPLPEGWEIRYTREGVRYFVDHNTRTTTFKDPRN

SEQ ID NO: 9 - protein, 870 aa, synthetic construct
MASASSSRAGVALPFEKSQLTLKVVSAKPKVHNRQPRINSYVEVAVDGLPSETKKTGKRIGSSELLWNEIIILNVTAQSHLDLKVWSCHTLRNELLGTASVNLSNVLKNNGGKMENMQLTLNLQTENKGSVVSGGELTIFLDGPTVDLGNVPNGSALTDGSQLPSRDSSGTAVAPENRHQPPSTNCFGGRSRTHRHSGASARTTPATGEQSPGARSRHRQPVKNSGHSGLANGTVNDEPTTATDPEEPSVVGVTSPPAAPLSVTPNPNTTSLPAPATPAEGEEPSTSGTQQLPAAAQAPDALPAGWEQRELPNGRVYYVDHNTKTTTWERPLPPGWEKRTDPRGRFYYVDHNTRTTTWQRPTAEYVRNYEQWQSQRNQLQGAMQHFSQRFLYQSSSASTDHDPLGPLPPGWEKRQDNGRVYYVNHNTRTTQWEDPRTQGMIQEPALPPGWEMKYTSEGVRYFVDHNTRTTTFKDPRPGFESGTKQGSPGAYDRSFRWKYHQFRFLCHSNALPSHVKISVSRQTLFEDSFQQIMNMKPYDLRRRLYIIMRGEEGLDYGGIAREWFFLLSHEVLNPMYCLFEYAGKNNYCLQINPASSINPDHLTYFRFIGRFIAMALYHGKFIDTGFTLPFYKRMLNKRPTLKDLESIDPEFYNSIVWIKENNLEECGLELYFIQDMEILGKVTTHELKEGGESIRVTEENKEEYIMLLTDWRFTRGVEEQTKAFLDGFNEVAPLEWLRYFDEKELELMLCGMQEIDMSDWQKSTIYRHYTKNSKQIQWFWQVVKEMDNEKRIRLLQFVTGTCRLPVGGFAELIGSNGPQKFCIDKVGKETWLPRSHTCFNRLDLPPYKSYEQLREKLLYAIEETEGFGQE

SEQ ID NO: 10 - protein, 34 aa, synthetic construct
DALPAGWEQRELPNGRVYYVDHNTKTTTWERPLP

SEQ ID NO: 11 - protein, 34 aa, synthetic construct; identical to SEQ ID NO: 10

SEQ ID NO: 12 - protein, 37 aa, synthetic construct
HDPLGPLPPGWEKRQDNGRVYYVNHNTRTTQWEDPRT

SEQ ID NO: 13 - protein, 34 aa, synthetic construct
PALPPGWEMKYTSEGVRYFVDHNTRTTTFKDPRP

SEQ ID NO: 14 - protein, 1319 aa, synthetic construct
MAQSLRLHFAARRSNTYPLSETSGDDLDSHVHMCFKRPTRISTSNVVQMKLTPRQTALAPLIKENVQSQERSSVPSSENVNKKSSCLQISLQPTRYSGYLQSSNVLADSDDASFTCILKDGIYSSAVVDNELNAVNDGHLVSSPAICSGSLSNFSTSDNGSYSSNGSDFGSCASITSGGSYTNSVISDSSSYTFPPSDDTFLGGNLPSDSTSNRSVPNRNTTPCEIFSRSTSTDPFVQDDLEHGLEIMKLPVSRNTKIPLKRYSSLVIFPRSPSTTRPTSPTSLCTLLSKGSYQTSHQFIISPSEIAHNEDGTSAKGFLSTAVNGLRLSKTICTPGEVRDIRPLHRKGSLQKKIVLSNNTPRQTVCEKSSEGYSCVSVHFTQRKAATLDCETTNGDCKPEMSEIKLNSDSEYIKLMHRTSACLPSSQNVDCQININGELERPHSQMNKNHGILRRSISLGGAYPNISCLSSLKHNCSKGGPSQLLIKFASGNEGKVDNLSRDSNRDCTNELSNSCKTRDDFLGQVDVPLYPLPTENPRLERPYTFKDFVLHPRSHKSRVKGYLRLKMTYLPKTSGSEDDNAEQAEELEPGWVVLDQPDAACHLQQQQEPSPLPPGWEERQDILGRTYYVNHESRRTQWKRPTPQDNLTDAENGNIQLQAQRAFTTRRQISEETESVDNRESSENWEIIREDEATMYSNQAFPSPPPSSNLDVPTHLAEELNARLTIFGNSAVSQPASSSNHSSRRGSLQAYTFEEQPTLPVLLPTSSGLPPGWEEKQDERGRSYYVDHNSRTTTWTKPTVQATVETSQLTSSQSSAGPQSQASTSDSGQQVTQPSEIEQGFLPKGWEVRHAPNGRPFFIDHNTKTTTWEDPRLKIPAHLRGKTSLDTSNDLGPLPPGWEERTHTDGRIFYINHNIKRTQWEDPRLENVAITGPAVPYSRDYKRKYEFFRRKLKKQNDIPNKFEMKLRRATVLEDSYRRIMGVKRADFLKARLWIEFDGEKGLDYGGVAREWFFLISKEMFNPYYGLFEYSATDNYTLQINPNSGLCNEDHLSYFKFIGRVAGMAVYHGKLLDGFFIRPFYKMMLHKPITLHDMESVDSEYYNSLRWILENDPTELDLRFIIDEELFGQTHQHELKNGGSEIVVTNKNKKEYIYLVIQWRFVNRIQKQMAAFKEGFFELIPQDLIKIFDENELELLMCGLGDVDVNDWREHTKYKNGYSANHQVIQWFWKAVLMMDSEKRIRLLQFVTGTSRVPMNGFAELYGSNGPQSFTVEQWGTPEKLPRAHTCFNRLDLPPYESFEELWDKLQMAIENTQGFDGVD

SEQ ID NO: 15 - protein, 34 aa, synthetic construct
SPLPPGWEERQDILGRTYYVNHESRRTQWKRPTP

SEQ ID NO: 16 - protein, 34 aa, synthetic construct
SGLPPGWEEKQDERGRSYYVDHNSRTTTWTKPTV

SEQ ID NO: 17 - protein, 34 aa, synthetic construct
GFLPKGWEVRHAPNGRPFFIDHNTKTTTWEDPRL

SEQ ID NO: 18 - protein, 34 aa, synthetic construct
GPLPPGWEERTHTDGRIFYINHNIKRTQWEDPRL

SEQ ID NO: 19 - protein, 955 aa, synthetic construct
MATGLGEPVYGLSEDEGESRILRVKVVSGIDLAKKDIFGASDPYVKLSLYVADENRELALVQTKTIKKTLNPKWNEEFYFRVNPSNHRLLFEVFDENRLTRDDFLGQVDVPLSHLPTEDPTMERPYTFKDFLLRPRSHKSRVKGFLRLKMAYMPKNGGQDEENSDQRDDMEHGWEVVDSNDSASQHQEELPPPPLPPGWEEKVDNLGRTYYVNHNNRTTQWHRPSLMDVSSESDNNIRQINQEAAHRRFRSRRHISEDLEPEPSEGGDVPEPWETISEEVNIAGDSLGLALPPPPASPGSRTSPQELSEELSRRLQITPDSNGEQFSSLIQREPSSRLRSCSVTDAVAEQGHLPPPSVAYVHTTPGLPSGWEERKDAKGRTYYVNHNNRTTTWTRPIMQLAEDGASGSATNSNNHLIEPQIRRPRSLSSPTVTLSAPLEGAKDSPVRRAVKDTLSNPQSPQPSPYNSPKPQHKVTQSFLPPGWEMRIAPNGRPFFIDHNTKTTTWEDPRLKFPVHMRSKTSLNPNDLGPLPPGWEERIHLDGRTFYIDHNSKITQWEDPRLQNPAITGPAVPYSREFKQKYDYFRKKLKKPADIPNRFEMKLHRNNIFEESYRRIMSVKRPDVLKARLWIEFESEKGLDYGGVAREWFFLLSKEMFNPYYGLFEYSATDNYTLQINPNSGLCNEDHLSYFTFIGRVAGLAVFHGKLLDGFFIRPFYKMMLGKQITLNDMESVDSEYYNSLKWILENDPTELDLMFCIDEENFGQTYQVDLKPNGSEIMVTNENKREYIDLVIQWRFVNRVQKQMNAFLEGFTELLPIDLIKIFDENELELLMCGLGDVDVNDWRQHSIYKNGYCPNHPVIQWFWKAVLLMDAEKRIRLLQFVTGTSRVPMNGFAELYGSNGPQLFTIEQWGSPEKLPRAHTCFNRLDLPPYETFEDLREKLLMAVENAQGFEGVD

SEQ ID NO: 20 - protein, 27 aa, synthetic construct
GWEEKVDNLGRTYYVNHNNRTTQWHRP

SEQ ID NO: 21 - protein, 29 aa, synthetic construct
PSGWEERKDAKGRTYYVNHNNRTTTWTRP

SEQ ID NO: 22 - protein, 31 aa, synthetic construct
PPGWEMRIAPNGRPFFIDHNTKTTTWEDPRL

SEQ ID NO: 23 - protein, 31 aa, synthetic construct
PPGWEERIHLDGRTFYIDHNSKITQWEDPRL

SEQ ID NO: 24 - protein, 756 aa, synthetic construct
MSNPGTRRNGSSIKIRLTVLCAKNLAKKDFFRLPDPFAKIVVDGSGQCHSTDTVKNTLDPKWNQHYDLYVGKTDSITISVWNHKKIHKKQGAGFLGCVRLLSNAISRLKDTGYQRLDLCKLNPSDTDAVRGQIVVSLQTRDRIGTGGSVDCRGLLENEGTVYEDSGPGRPLSCFMEEPAPYTDSTGAAAGGGNCRFVESPSQDQRLQAQRLRNPDVRGSLQTPQNRPHGHQSPELPEGYEQRTTVQGQVYFLHTQTGVSTWHDPRIPSPSGTIPGGDAAFLYEFLLQGHTSEPRDLNSVNCDELGPLPPGWEVRSTVSGRIYFVDHNNRTTQFTDPRLHHIMNHQCQLKEPSQPLPLPSEGSLEDEELPAQRYERDLVQKLKVLRHELSLQQPQAGHCRIEVSREEIFEESYRQIMKMRPKDLKKRLMVKFRGEEGLDYGGVAREWLYLLCHEMLNPYYGLFQYSTDNIYMLQINPDSSINPDHLSYFHFVGRIMGLAVFHGHYINGGFTVPFYKQLLGKPIQLSDLESVDPELHKSLVWILENDITPVLDHTFCVEHNAFGRILQHELKPNGRNVPVTEENKKEYVRLYVNWRFMRGIEAQFLALQKGFNELIPQHLLKPFDQKELELIIGGLDKIDLNDWKSNTRLKHCVADSNIVRWFWQAVETFDEERRARLLQFVTGSTRVPLQGFKALQGSTGAAGPRLFTIHLIDANTDNLPKAHTCFNRIDIPPYESYEKLYEKLLTAVEETCGFAVE

SEQ ID NO: 25 - protein, 34 aa, synthetic construct
PELPEGYEQRTTVQGQVYFLHTQTGVSTWHDPRI

SEQ ID NO: 26 - protein, 34 aa, synthetic construct
GPLPPGWEVRSTVSGRIYFVDHNNRTTQFTDPRL

SEQ ID NO: 27 - protein, 748 aa, synthetic construct
MSNPGGRRNGPVKLRLTVLCAKNLVKKDFFRLPDPFAKVVVDGSGQCHSTDTVKNTLDPKWNQHYDLYIGKSDSVTISVWNHKKIHKKQGAGFLGCVRLLSNAINRLKDTGYQRLDLCKLGPNDNDTVRGQIVVSLQSRDRIGTGGQVVDCSRLFDNDLPDGWEERRTASGRIQYLNHITRTTQWERPTRPASEYSSPGRPLSCFVDENTPISGTNGATCGQSSDPRLAERRVRSQRHRNYMSRTHLHTPPDLPEGYEQRTTQQGQVYFLHTQTGVSTWHDPRVPRDLSNINCEELGPLPPGWEIRNTATGRVYFVDHNNRTTQFTDPRLSANLHLVLNRQNQLKDQQQQQVVSLCPDDTECLTVPRYKRDLVQKLKILRQELSQQQPQAGHCRIEVSREEIFEESYRQVMKMRPKDLWKRLMIKFRGEEGLDYGGVAREWLYLLSHEMLNPYYGLFQYSRDDIYTLQINPDSAVNPEHLSYFHFVGRIMGMAVFHGHYIDGGFTLPFYKQLLGKSITLDDMELVDPDLHNSLVWILENDITGVLDHTFCVEHNAYGEIIQHELKPNGKSIPVNEENKKEYVRLYVNWRFLRGIEAQFLALQKGFNEVIPQHLLKTFDEKELELIICGLGKIDVNDWKVNTRLKHCTPDSNIVKWFWKAVEFFDEERRARLLQFVTGSSRVPLQGFKALQGAAGPRLFTIHQIDACTNNLPKAHTCFNRIDIPPYESYEKLYEKLLTAIEETCGFAVE

SEQ ID NO: 28 - protein, 34 aa, synthetic construct
NDLPDGWEERRTASGRIQYLNHITRTTQWERPTR

SEQ ID NO: 29 - protein, 34 aa, synthetic construct
PDLPEGYEQRTTQQGQVYFLHTQTGVSTWHDPRV

SEQ ID NO: 30 - protein, 34 aa, synthetic construct
GPLPPGWEIRNTATGRVYFVDHNNRTTQFTDPRL

SEQ ID NO: 31 - protein, 903 aa, synthetic construct
MSDSGSQLGSMGSLTMKSQLQITVISAKLKENKKNWFGPSPYVEVTVDGQSKKTEKCNNTNSPKWKQPLTVIVTPVSKLHFRVWSHQTLKSDVLLGTAALDIYETLKSNNMKLEEVVVTLQLGGDKEPTETIGDLSICLDGLQLESEVVTNGETTCSENGVSLCLPRLECNSAISAHCNLCLPGLSDSPISASRVAGFTGASQNDDGSRSKDETRVSTNGSDDPEDAGAGENRRVSGNNSPSLSNGGFKPSRPPRPSRPPPPTPRRPASVNGSPSATSESDGSSTGSLPPTNTNTNTSEGATSGLIIPLTISGGSGPRPLNPVTQAPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQSKEFDPLGPLPPGWEKRTDSNGRVYFVNHNTRITQWEDPRSQGQLNEKPLPEGWEMRFTVDGIPYFVDHNRRTTTYIDPRTGKSALDNGPQIAYVRDFKAKVQYFRFWCQQLAMPQHIKITVTRKTLFEDSFQQIMSFSPQDLRRRLWVIFPGEEGLDYGGVAREWFFLLSHEVLNPMYCLFEYAGKDNYCLQINPASYINPDHLKYFRFIGRFIAMALFHGKFIDTGFSLPFYKRILNKPVGLKDLESIDPEFYNSLIWVKENNIEECDLEMYFSVDKEILGEIKSHDLKPNGGNILVTEENKEEYIRMVAEWRLSRGVEEQTQAFFEGFNEILPQQYLQYFDAKELEVLLCGMQEIDLNDWQRHAIYRHYARTSKQIMWFWQFVKEIDNEKRMRLLQFVTGTCRLPVGGFADLMGSNGPQKFCIEKVGKENWLPRSHTCFNRLDLPPYKSYEQLKEKLLFAIEETEGFGQE

SEQ ID NO: 32 - protein, 34 aa, synthetic construct
APLPPGWEQRVDQHGRVYYVDHVEKRTTWDRPEP

SEQ ID NO: 33 - protein, 34 aa, synthetic construct
EPLPPGWERRVDNMGRIYYVDHFTRTTTWQRPTL

SEQ ID NO: 34 - protein, 34 aa, synthetic construct
GPLPPGWEKRTDSNGRVYFVNHNTRITQWEDPRS

SEQ ID NO: 35 - protein, 34 aa, synthetic construct
KPLPEGWEMRFTVDGIPYFVDHNRRTTTYIDPRT

SEQ ID NO: 36 - protein, 1606 aa, synthetic construct
MLLHLCSVKNLYQNRFLGLAAMASPSRNSQSRRRCKEPLRYSYNPDQFHNMDLRGGPHDGVTIPRSTSDTDLVTSDSRSTLMVSSSYYSIGHSQDLVIHWDIKEEVDAGDWIGMYLIDEVLSENFLDYKNRGVNGSHRGQIIWKIDASSYFVEPETKICFKYYHGVSGALRATTPSVTVKNSAAPIFKSIGADETVQGQGSRRLISFSLSDFQAMGLKKGMFFNPDPYLKISIQPGKHSIFPALPHHGQERRSKIIGNTVNPIWQAEQFSFVSLPTDVLEIEVKDKFAKSRPIIKRFLGKLSMPVQRLLERHAIGDRVVSYTLGRRLPTDHVSGQLQFRFEITSSIHPDDEEISLSTEPESAQIQDSPMNNLMESGSGEPRSEAPESSESWKPEQLGEGSVPDGPGNQSIELSRPAEEAAVITEAGDQGMVSVGPEGAGELLAQVQKDIQPAPSAEELAEQLDLGEEASALLLEDGEAPASTKEEPLEEEATTQSRAGREEEEKEQEEEGDVSTLEQGEGRLQLRASVKRKSRPCSLPVSELETVIASACGDPETPRTHYIRIHTLLHSMPSAQGGSAAEEEDGAEEESTLKDSSEKDGLSEVDTVAADPSALEEDREEPEGATPGTAHPGHSGGHFPSLANGAAQDGDTHPSTGSESDSSPRQGGDHSCEGCDASCCSPSCYSSSCYSTSCYSSSCYSASCYSPSCYNGNRFASHTRFSSVDSAKISESTVFSSQDDEEEENSAFESVPDSMQSPELDPESTNGAGPWQDELAAPSGHVERSPEGLESPVAGPSNRREGECPILHNSQPVSQLPSLRPEHHHYPTIDEPLPPNWEARIDSHGRVFYVDHVNRTTTWQRPTAAATPDGMRRSGSIQQMEQLNRRYQNIQRTIATERSEEDSGSQSCEQAPAGGGGGGGSDSEAESSQSSLDLRREGSLSPVNSQKITLLLQSPAVKFITNPEFFTVLHANYSAYRVFTSSTCLKHMILKVRRDARNFERYQHNRDLVNFINMFADTRLELPRGWEIKTDQQGKSFFVDHNSRATTFIDPRIPLQNGRLPNHLTHRQHLQRLRSYSAGEASEVSRNRGASLLARPGHSLVAAIRSQHQHESLPLAYNDKIVAFLRQPNIFEMLQERQPSLARNHTLREKIHYIRTEGNHGLEKLSCDADLVILLSLFEEEIMSYVPLQAAFHPGYSFSPRCSPCSSPQNSPGLQRASARAPSPYRRDFEAKLRNFYRKLEAKGFGQGPGKIKLIIRRDHLLEGTFNQVMAYSRKELQRNKLYVTFVGEEGLDYSGPSREFFFLLSQELFNPYYGLFEYSANDTYTVQISPMSAFVENHLEWFRFSGRILGLALIHQYLLDAFFTRPFYKALLRLPCDLSDLEYLDEEFHQSLQWMKDNNITDILDLTFTVNEEVFGQVTERELKSGGANTQVTEKNKKEYIERMVKWRVERGVVQQTEALVRGFYEVVDSRLVSVFDARELELVIAGTAEIDLNDWRNNTEYRGGYHDGHLVIRWFWAAVERFNNEQRLRLLQFVTGTSSVPYEGFAALRGSNGLRRFCIEKWGKITSLPRAHTCFNRLDLPPYPSYSMLYEKLLTAVEETSTFGLE

SEQ ID NO: 37 - protein, 33 aa, synthetic construct
PLPPNWEARIDSHGRVFYVDHVNRTTTWQRPTA

SEQ ID NO: 38 - protein, 34 aa, synthetic construct
LELPRGWEIKTDQQGKSFFVDHNSRATTFIDPRI

SEQ ID NO: 39 - protein, 1572 aa, synthetic construct
MASSAREHLLFVRRRNPQMRYTLSPENLQSLAAQSSMPENMTLQRANSDTDLVTSESRSSLTASMYEYTLGQAQNLIIFWDIKEEVDPSDWIGLYHIDENSPANFWDSKNRGVTGTQKGQIVWRIEPGPYFMEPEIKICFKYYHGISGALRATTPCITVKNPAVMMGAEGMEGGASGNLHSRKLVSFTLSDLRAVGLKKGMFFNPDPYLKMSIQPGKKSSFPTCAHHGQERRSTIISNTTNPIWHREKYSFFALLTDVLEIEIKDKFAKSRPIIKRFLGKLTIPVQRLLERQAIGDQMLSYNLGRRLPADHVSGYLQFKVEVTSSVHEDASPEAVGTILGVNSVNGDLGSPSDDEDMPGSHHDSQVCSNGPVSEDSAADGTPKHSFRTSSTLEIDTEELTSTSSRTSPPRGRQDSLNDYLDAIEHNGHSRPGTATCSERSMGASPKLRSSFPTDTRLNAMLHIDSDEEDHEFQQDLGYPSSLEEEGGLIMFSRASRADDGSLTSQTKLEDNPVENEEASTHEAASFEDKPENLPELAESSLPAGPAPEEGEGGPEPQPSADQGSAELCGSQEVDQPTSGADTGTSDASGGSRRAVSETESLDQGSEPSQVSSETEPSDPARTESVSEASTRPEGESDLECADSSCNESVTTQLSSVDTRCSSLESARFPETPAFSSQEEEDGACAAEPTSSGPAEGSQESVCTAGSLPVVQVPSGEDEGPGAESATVPDQEELGEVWQRRGSLEGAAAAAESPPQEEGSAGEAQGTCEGATAQEEGATGGSQANGHQPLRSLPSVRQDVSRYQRVDEALPPNWEARIDSHGRIFYVDHVNRTTTWQRPTAPPAPQVLQRSNSIQQMEQLNRRYQSIRRTMTNERPEENTNAIDGAGEEADFHQASADFRRENILPHSTSRSRITLLLQSPPVKFLISPEFFTVLHSNPSAYRMFTNNTCLKHMITKVRRDTHHFERYQHNRDLVGFLNMFANKQLELPRGWEMKHDHQGKAFFVDHNSRTTTFIDPRLPLQSSRPTSALVHRQHLTRQRSHSAGEVGEDSRHAGPPVLPRPSSTFNTVSRPQYQDMVPVAYNDKIVAFLRQPNIFEILQERQPDLTRNHSLREKIQFIRTEGTPGLVRLSSDADLVMLLSLFEEEIMSYVPPHALLHPSYCQSPRGSPVSSPQNSPGTQRANARAPAPYKRDFEAKLRNFYRKLETKGYGQGPGKLKLIIRRDHLLEDAFNQIMGYSRKDLQRNKLYVTFVGEEGLDYSGPSREFFFLVSRELFNPYYGLFEYSANDTYTVQISPMSAFVDNHHEWFRFSGRILGLALIHQYLLDAFFTRPFYKALLRILCDLSDLEYLDEEFHQSLQWMKDNDIHDILDLTFTVNEEVFGQITERELKPGGANIPVTEKNKKEYIERMVKWRIERGVVQQTESLVRGFYEVVDARLVSVFDARELELVIAGTAEIDLSDWRNNTEYRGGYHDNHIVIRWFWAAVERFNNEQRLRLLQFVTGTSSIPYEGFASLRGSNGPRRFCVEKWGKITALPRAHTCFNRLDLPPYPSFSMLYEKLLTAVEETSTFGLE

SEQ ID NO: 40 - protein, 34 aa, synthetic construct
EALPPNWEARIDSHGRIFYVDHVNRTTTWQRPTA

SEQ ID NO: 41 - protein, 34 aa, synthetic construct
LELPRGWEMKHDHQGKAFFVDHNSRTTTFIDPRL

SEQ ID NO: 42 - protein, 433 aa, Homo sapiens
MGRVQLFEISLSHGRVVYSPGEPLAGTVRVRLGAPLPFRAIRVTCIGSCGVSNKANDTAWVVEEGYFNSSLSLADKGSLPAGEHSFPFQFLLPATAPTSFEGPFGKIVHQVRAAIHTPRFSKDHKCSLVFYILSPLNLNSIPDIEQPNVASATKKFSYKLVKTGSVVLTASTDLRGYVVGQALQLHADVENQSGKDTSPVVASLLQKVSYKAKRWIHDVRTIAEVEGAGVKAWRRAQWHEQILVPALPQSALPGCSLIHIDYYLQVSLKAPEATVTLPVFIGNIAVNHAPVSPRPGLGLPPGAPPLVVPSAPPQEEAEAEAAAGGPHFLDPVFLSTKSHSQRQPLLATLSSVPGAPEPCPQDGSPASHPLHPPLCISTGATVPYFAEGSGGPVPTTSTLILPPEYSSWGYPYEAPPSYEQSCGGVEPSLTPES

SEQ ID NO: 43 - protein, 434 aa, Mus musculus
MGRVQLFEIRLSQGRVVYGPGEPLAGTVHLRLGAPLPFRAIRVTCMGSCGVSTKANDGAWVVEESYFNSSLSLADKGSLPAGEHNFPFQFLLPATAPTSFEGPFGKIVHQVRASIDTPRFSKDHKCSLVFYILSPLNLNSIPDIEQPNVASTTKKFSYKLVKTGNVVLTASTDLRGYVVGQVLRLQADIENQSGKDTSPVVASLLQKVSYKAKRWIYDVRTIAEVEGTGVKAWRRAQWQEQILVPALPQSALPGCSLIHIDYYLQVSMKAPEATVTLPLFVGNIAVNQTPLSPCPGRESSPGTLSLVVPSAPPQEEAEAVASGPHFSDPVSLSTKSHSQQQPLSAPLGSVSVTTTEPWVQVGSPARHSLHPPLCISIGATVPYFAEGSAGPVPTTSALILPPEYSSWGYPYEAPPSYEQSCGAAGTDLGLIPGS

SEQ ID NO: 44 - protein, 433 aa, Mus musculus
MGRVQLFEIRLSQGRVVYGPGEPLAGTVHLRLGAPLPFRAIRVTCMGSCGVSTKANDGAWVVEESYFNSSLSLADKGSLPAGEHNFPFQFLLPATAPTSFEGPFGKIVHQVRASIDTPRFSKDHKCSLVFYILSPLNLNSIPDIEQPNVASTTKKFSYKLVKTGNVVLTASTDLRGYVVGQVLRLQADIENQSGKDTSPVVASLLQVSYKAKRWIYDVRTIAEVEGTGVKAWRRAQWQEQILVPALPQSALPGCSLIHIDYYLQVSMKAPEATVTLPLFVGNIAVNQTPLSPCPGRESSPGTLSLVVPSAPPQEEAEAVASGPHFSDPVSLSTKSHSQQQPLSAPLGSVSVTTTEPWVQVGSPARHSLHPPLCISIGATVPYFAEGSAGPVPTTSALILPPEYSSWGYPYEAPPSYEQSCGAAGTDLGLIPGS

SEQ ID NO: 45 - protein, 390 aa, Homo sapiens
MAVSESQLKKMVSKYKYRDLTVRETVNVITLYKDLKPVLDSYVFNDGSSRELMNLTGTIPVPYRGNTYNIPICLWLLDTYPYNPPICFVKPTSSMTIKTGKHVDANGKIYLPYLHEWKHPQSDLLGLIQVMIVVFGDEPPVFSRPISASYPPYQATGPPNTSYMPGMPGGISPYPSGYPPNPSGYPGCPYPPGGPYPATTSSQYPSQPPVTTVGPSRDGTISEDTIRASLISAVSDKLRWRMKEEMDRAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 46 - protein, 391 aa, Mus musculus
MAVSESQLKKMMSKYKYRDLTVRQTVNVIAMYKDLKPVLDSYVFNDGSSRELVNLTGTIPVRYRGNIYNIPICLWLLDTYPYNPPICFVKPTSSMTIKTGKHVDANGKIYLPYLHDWKHPRSELLELIQIMIVIFGEEPPVFSRPTVSASYPPYTATGPPNTSYMPGMPSGISAYPSGYPPNPSGYPGCPYPPAGPYPATTSSQYPSQPPVTTVGPSRDGTISEDTIRASLISAVSDKLRWRMKEEMDGAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 47 - protein, 391 aa, Rattus norvegicus
MAVSESQLKKMMSKYKYRDLTVRQTVNVIAMYKDLKPVLDSYVFNDGSSRELVNLTGTIPVRYRGNIYNIPICLWLLDTYPYNPPICFVKPTSSMTIKTGKHVDANGKIYLPYLHDWKHPRSELLELIQIMIVIFGEEPPVFSRPTVSASYPPYTAAGPPNTSYLPSMPSGISAYPSGYPPNPSGYPGCPYPPAGPYPATTSSQYPSQPPVTTAGPSRDGTISEDTIRASLISAVSDKLRWRMKEEMDGAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 48 - DNA, 3475 bp, Homo sapiens
  source 1..3475: mol_type = unassigned DNA
gaattcgcggccgcgtcgaccgcttctgtggccacggcagatgaaacagaaaggctaaagagggctggagtcaggggacttctcttccaccagcttcacggtgatgatatggcatctgccagctctagccgggcaggagtggccctgccttttgagaagtctcagctcactttgaaagtggtgtccgcaaagcccaaggtgcataatcgtcaacctcgaattaactcctacgtggaggtggcggtggatggactccccagtgagaccaagaagactgggaagcgcattgggagctctgagcttctctggaatgagatcatcattttgaatgtcacggcacagagtcatttagatttaaaggtctggagctgccataccttgagaaatgaactgctaggcaccgcatctgtcaacctctccaacgtcttgaagaacaatgggggcaaaatggagaacatgcagctgaccctgaacctgcagacggagaacaaaggcagcgttgtctcaggcggaaaactgacaattttcctggacgggccaactgttgatctgggaaatgtgcctaatggcagtgccctgacagatggatcacagctgccttcgagagactccagtggaacagcagtagctccagagaaccggcaccagccccccagcacaaactgctttggtggaagatcccggacgcacagacattcgggtgcttcagccagaacaaccccagcaaccggcgagcaaagccccggtgctcggagccggcaccgccagcccgtcaagaactcaggccacagtggcttggccaatggcacagtgaatgatgaacccacaacagccactgatcccgaagaaccttccgttgttggtgtgacgtccccacctgctgcacccttgagtgtgaccccgaatcccaacacgacttctctccctgccccagccacaccggctgaaggagaggaacccagcacttcgggtacacagcagctcccagcggctgcccaggcccccgacgctctgcctgctggatgggaacagcgagagctgcccaacggacgtgtctattatgttgaccacaataccaagaccaccacctgggagcggccccttcctccaggctgggaaaaacgcacagatccccgaggcaggttttactatgtggatcacaatactcggaccaccacctggcagcgtccgaccgcggagtacgtgcgcaactatgagcagtggcagtcgcagcggaatcagctccagggggccatgcagcacttcagccaaagattcctataccagttttggagtgcttcgactgaccatgatcccctgggccccctccctcctggttgggagaaaagacaggacaatggacgggtgtattacgtgaaccataacactcgcacgacccagtgggaggatccccggacccaggggatgatccaggaaccagctttgcccccaggatgggagatgaaatacaccagcgagggggtgcgatactttgtggaccacaatacccgcaccaccacctttaaggatcctcgcccggggtttgagtcggggacgaagcaaggttcccctggtgcttatgaccgcagttttcggtggaagtatcaccagttccgtttcctctgccattcaaatgccctacctagccacgtgaagatcagcgtttccaggcagacgcttttcgaagattccttccaacagatcatgaacatgaaaccctatgacctgcgccgccggctttacatcatcatgcgtggcgaggagggcctggactatgggggcatcgccagagagtggtttttcctcctgtctcacgaggtgctcaaccctatgtattgtttatttgaatatgccggaaagaacaattactgcctgcagatcaaccccgcctcctccatcaacccggaccacctcacctactttcgctttataggcagattcatcgccatggcgctgtaccatggaaagttcatcgacacgggcttcaccctccctttctacaagcggatgctcaataagagaccaaccctgaaagacctggagtccattgaccctgagttctacaactccattgtctggatcaaagagaacaacctggaagaatgtggcctggagctgtacttcatccaggacatggagatactgggcaaggtgacgacccacgagctgaaggagggcggcgagagcatccgggtcacggaggagaacaaggaagagtacatcatgctgctgactgactggcgtttcacccgaggcgtggaagagcagaccaaagccttcctggatggcttcaacgaggtggccccgctggagtggctgcgctactttgacgagaaagagctggagctgatgctgtgcggcatgcaggagatagacatgagcgactggcagaagagcaccatctaccggcactacaccaagaacagcaagcagatccagtggttctggcaggtggtgaaggagatggacaacgagaagaggatccggctgctgcagtttgtcaccggtacctgccgcctgcccgtcgggggatttgccgaactcatcggtagcaacggaccacagaagttttgcattgacaaagttggcaaggaaacctggctgcccagaagccacacctgcttcaaccgtctggatcttccaccctacaagagctacgaacagctgagagagaagctgctgtatgccattgaggagaccgagggctttggacaggagtaaccgaggccgcccctcccacgccccccagcgcacatgtagtcctgagtcctccctgcctgagaggccactggccccgcagcccttgggaggcccccgtggatgtggccctgtgtgggaccacactgtcatctcgctgctggcagaaaagcctgatcccaggaggccctgcagttcccccgacccgcggatggcagtctggaataaagccccctagttgcctttggccccacctttgcaaagttccagagggctgaccctctctgcaaaactctcccctgtcctctagaccccaccctgggtgtatgtgagtgtgcaagggaaggtgttgcatccccaggggctgccgcagaggccggagacctcctggactagttcggcgaggagactggccactgggggtggctgttcgggactgagagcgccaagggtctttgccagcaaaggaggttctgcctgtaattgagcctctctgatgatggagatgaagtgaaggtctgagggacgggccctggggctaggccatctctgcctgcctccctagcaggcgccagcggtggaggctgagtcgcaggacacatgccggccagttaattcattctcagcaaatgaaggtttgtctaagctgcctgggtatccacgggacaaaaacagcaaactccctccagactttgtccatgttataaacttgaaagttggttgttgtttgttaggtttgccaggtttttttgtttacgcctgctgtcactttcctgtc

SEQ ID NO: 49 - DNA, 3475 bp, Homo sapiens; identical to SEQ ID NO: 48
  source 1..3475: mol_type = unassigned DNA

SEQ ID NO: 50 - DNA, 2914 bp, Homo sapiens
  source 1..2914: mol_type = unassigned DNA
acagttgcctgccctgggcgggggcgagcgcgtccggtttgctggaagcgttcggaaatggcaacttgcgcggtggaggtgttcgggctcctggaggacgaggaaaattcacgaattgtgagagtaagagttatagccggaataggccttgccaagaaggatatattgggagctagtgatccttacgtgagagtgacgttatatgacccaatgaatggagttcttacaagtgtgcaaacaaaaaccattaaaaagagtttgaatccaaagtggaatgaagaaatattattcagagttcatcctcagcagcaccggcttctttttgaagtgtttgacgaaaaccgattgacaagagatgatttcctaggtcaagtggatgttccactttatccattaccgacagaaaatccaagattggagagaccatatacatttaaggattttgttcttcatccaagaagtcacaaatcaagagttaaaggttatctgagactaaaaatgacttatttacctaaaaccagtggctcagaagatgataatgcagaacaggctgaggaattagagcctggctgggttgttttggaccaaccagatgctgcttgccatttgcagcaacaacaagaaccttctcctctacctccagggtgggaagagaggcaggatatccttggaaggacctattatgtaaaccatgaatctagaagaacacagtggaaaagaccaacccctcaggacaacctaacagatgctgagaatggcaacattcaactgcaagcacaacgtgcatttaccaccaggcggcagatatccgaggaaacagaaagtgttgacaaccaagagtcttccgagaactgggaaattataagagaagatgaagccaccatgtatagcagccaggccttcccatcacctccaccgtcaagtaacttggatgttccaactcatcttgcagaagaattgaatgccagactcaccatttttggaaattcagccgtgagccagccagcatcgagctcaaatcattccagcagaagaggcagcttacaagcctatacttttgaggaacaacctacacttcctgtgcttttgcctacttcatctggattaccaccaggttgggaagaaaaacaagatgaaagaggaagatcatattatgtagatcacaattccagaacgactacttggacaaagcccactgtacaggccacagtggagaccagtcagctgacctcaagccagagttctgcaggccctcaatcacaagcctccaccagtgattcaggccagcaggtgacccagccatctgaaattgagcaaggattccttcctaaaggctgggaagtccggcatgcaccaaatgggaggcctttctttattgaccacaacactaaaaccaccacctgggaagatccaagattgaaaattccagcccatctgagaggaaagacatcacttgatacttccaatgatctagggcctttacctccaggatgggaagagagaactcacacagatggaagaatcttctacataaatcacaatataaaaagaacacaatgggaagatcctcggttggagaatgtagcaataactggaccagcagtgccctactccagggattacaaaagaaagtatgagttcttccgaagaaagttgaagaagcagaatgacattccaaacaaatttgaaatgaaacttcgccgagcaactgttcttgaagactcttaccggagaattatgggtgtcaagagagcagacttcctgaaggctcgactgtggattgagtttgatggtgaaaagggattggattatggaggagttgccagagaatggttcttcctgatctcaaaggaaatgtttaacccttattatgggttgtttgaatattctgctacggacaattataccctacagataaatccaaactctggattgtgtaacgaagatcacctctcttacttcaagtttattggtcgggtagctggaatggcagtttatcatggcaaactgttggatggttttttcatccgcccattttacaagatgatgcttcacaaaccaataacccttcatgatatggaatctgtggatagtgaatattacaattccctaagatggattcttgaaaatgacccaacagaattggacctcaggtttatcatagatgaagaactttttggacagacacatcaacatgagctgaaaaatggtggatcagaaatagttgtcaccaataagaacaaaaaggaatatatttatcttgtaatacaatggcgatttgtaaaccgaatccagaagcaaatggctgcttttaaagagggattctttgaactaataccacaggatctcatcaaaatttttgatgaaaatgaactagagcttcttatgtgtggaccgggagatgttgatgtgaatgactggagggaacatacaaagtataaaaatggctacagtgcaaatcatcaggttatacagtggttttggaaggctgttttaatgatggattcagaaaaaagaataagattacttcagtttgtcactggcacatctcgggtgcctatgaatggatttgctgaactatacggttcaaatggaccacagtcatttacagttgaacagtggggtactcctgaaaagctgccaagagctcatacctgttttaatcgcctggacttgccaccttatgaatcatttgaagaattatgggataaacttcagatggcaattgaaaacacccagggctttgatggagttgattagattacaaataacaatctgtagtgtttttactgccatagttttataaccaaaatcttgacttaaaattttccggggaactactaaaatgtggccactgagtcttcccagatcttgaagaaaatcatataaaaagcatttgaagaaatagtacgac

SEQ ID NO: 51 - DNA, 2868 bp, Homo sapiens
  source 1..2868: mol_type = unassigned DNA
atggcgaccgggctcggggagccggtctatggactttccgaagacgagggagagtcccgtattctcagagtaaaagttgtttctggaattgatctcgccaaaaaggacatctttggagccagtgatccgtatgtgaaactttcattgtacgtagcggatgagaatagagaacttgctttggtccagacaaaaacaattaaaaagacactgaacccaaaatggaatgaagaattttatttcagggtaaacccatctaatcacagactcctatttgaagtatttgacgaaaatagactgacacgagacgacttcctgggccaggtggacgtgccccttagtcaccttccgacagaagatccaaccatggagcgaccctatacatttaaggactttctcctcagaccaagaagtcataagtctcgagttaagggatttttgcgattgaaaatggcctatatgccaaaaaatggaggtcaagatgaagaaaacagtgaccagagggatgacatggagcatggatgggaagttgttgactcaaatgactcggcttctcagcaccaagaggaacttcctcctcctcctctgcctcccgggtgggaagaaaaagtggacaatttaggccgaacttactatgtcaaccacaacaaccggaccactcagtggcacagaccaagcctgatggacgtgtcctcggagtcggacaataacatcagacagatcaaccaggaggcagcacaccggcgcttccgctcccgcaggcacatcagcgaagacttggagcccgagccctcggagggcggggatgtccccgagccttgggagaccatttcagaggaagtgaatatcgctggagactctctcggtctggctctgcccccaccaccggcctccccaggatctcggaccagccctcaggagctgtcagaggaactaagcagaaggcttcagatcactccagactccaatggggaacagttcagctctttgattcaaagagaaccctcctcaaggttgaggtcatgcagtgtcaccgacgcagttgcagaacagggccatctaccaccgccatcagtggcctatgtacataccacgccgggtctgccttcaggctgggaagaaagaaaagatgctaaggggcgcacatactatgtcaatcataacaatcgaaccacaacttggactcgacctatcatgcagcttgcagaagatggtgcgtccggatcagccacaaacagtaacaaccatctaatcgagcctcagatccgccggcctcgtagcctcagctcgccaacagtaactttatctgccccgctggagggtgccaaggactcacccgtacgtcgggctgtgaaagacaccctttccaacccacagtccccacagccatcaccttacaactcccccaaaccacaacacaaagtcacacagagcttcttgccacccggctgggaaatgaggatagcgccaaacggccggcccttcttcattgatcataacacaaagactacaacctgggaagatccacgtttgaaatttccagtacatatgcggtcaaagacatctttaaaccccaatgaccttggcccccttcctcctggctgggaagaaagaattcacttggatggccgaacgttttatattgatcataatagcaaaattactcagtgggaagacccaagactgcagaacccagctattactggtccggctgtcccttactccagagaatttaagcagaaatatgactacttcaggaagaaattaaagaaacctgctgatatccccaataggtttgaaatgaaacttcacagaaataacatatttgaagagtcctatcggagaattatgtccgtgaaaagaccagatgtcctaaaagctagactgtggattgagtttgaatcagagaaaggtcttgactatgggggtgtggccagagaatggttcttcttactgtccaaagagatgttcaacccctactacggcctctttgagtactctgccacggacaactacacccttcagatcaaccctaattcaggcctctgtaatgaggatcatttgtcctacttcacttttattggaagagttgctggtctggccgtatttcatgggaagctcttagatggtttcttcattagaccattttacaagatgatgttgggaaagcagataaccctgaatgacatggaatctgtggatagtgaatattacaactctttgaaatggatcctggagaatgaccctactgagctggacctcatgttctgcatagacgaagaaaactttggacagacatatcaagtggatttgaagcccaatgggtcagaaataatggtcacaaatgaaaacaaaagggaatatatcgacttagtcatccagtggagatttgtgaacagggtccagaagcagatgaacgccttcttggagggattcacagaactacttcctattgatttgattaaaatttttgatgaaaatgagctggagttgctcatgtgcggcctcggtgatgtggatgtgaatgactggagacagcattctatttacaagaacggctactgcccaaaccaccccgtcattcagtggttctggaaggctgtgctactcatggacgccgaaaagcgtatccggttactgcagtttgtcacagggacatcgcgagtacctatgaatggatttgccgaactttatggttccaatggtcctcagctgtttacaatagagcaatggggcagtcctgagaaactgcccagagctcacacatgctttaatcgccttgacttacctccatatgaaacctttgaagatttacgagagaaacttctcatggccgtggaaaatgctcaaggatttgaaggggtggattaa

SEQ ID NO: 52 - DNA, 2274 bp, Homo sapiens
  source 1..2274: mol_type = unassigned DNA
atgtcgaaccccgggacacgcaggaacggctccagcatcaagatccgtctgacagtgttatgtgccaagaaccttgcaaagaaagacttcttcaggctccctgacccttttgcaaagattgtcgtggatgggtctgggcagtgccactcaaccgacactgtgaaaaacacattggacccaaagtggaaccagcactatgatctatatgttgggaaaacggattcgataaccattagcgtgtggaaccataagaaaattcacaagaaacagggagctggcttcctgggctgtgtgcggctgctctccaatgccatcagcagattaaaagataccggataccagcgtttggatctatgcaaactaaacccctcagatactgatgcagttcgtggccagatagtggtcagtttacagacacgagacagaataggaaccggcggctcggtggtggactgcagaggactgttagaaaatgaaggaacggtgtatgaagactccgggcctgggaggccgctcagctgcttcatggaggaaccagccccttacacagatagcaccggtgctgctgctggaggagggaattgcaggttcgtggagtccccaagtcaagatcaaagacttcaggcacagcggcttcgaaaccctgatgtgcgaggttcactacagacgccccagaaccgaccacacggccaccagtccccggaactgcccgaaggctacgaacaaagaacaacagtccagggccaagtttactttttgcatacacagactggagttagcacgtggcacgaccccaggataccaagtccctcggggaccattcctgggggagatgcagcttttctatacgaattccttctacaaggccatacatctgagcccagagaccttaacagtgtgaactgtgatgaacttggaccactgccgccaggctgggaagtcagaagtacagtttctgggaggatatattttgtagatcataataaccgaacaacccagtttacagacccaaggttacaccacatcatgaatcaccagtgccaactcaaggagcccagccagccgctgccactgcccagtgagggctctctggaggacgaggagcttcctgcccagagatacgaaagagatctagtccagaagctgaaagtcctcagacacgaactgtcgcttcagcagccccaagctggtcattgccgcatcgaagtgtccagagaagaaatctttgaggagtcttaccgccagataatgaagatgcgaccgaaagacttgaaaaaacggctgatggtgaaattccgtggggaagaaggtttggattacggtggtgtggccagggagtggctttacttgctgtgccatgaaatgctgaatccttattacgggctcttccagtattctacggacaatatttacatgttgcaaataaatccggattcttcaatcaaccccgaccacttgtcttatttccactttgtggggcggatcatggggctggctgtgttccatggacactacatcaacgggggcttcacagtgcccttctacaagcagctgctggggaagcccatccagctctcagatctggaatctgtggacccagagctgcataagagcttggtgtggatcctagagaacgacatcacgcctgtactggaccacaccttctgcgtggaacacaacgccttcgggcggatcctgcagcatgaactgaaacccaatggcagaaatgtgccagtcacagaggagaataagaaagaatacgtccggttgtatgtaaactggaggtttatgagaggaatcgaagcccagttcttagctctgcagaaggggttcaatgagctcatccctcaacatctgctgaagccttttgaccagaaggaactggagctgatcataggcggcctggataaaatagacttgaacgactggaagtcgaacacgcggctgaagcactgtgtggccgacagcaacatcgtgcggtggttctggcaagcggtggagacgttcgatgaagaaaggagggccaggctcctgcagtttgtgactgggtccacgcgagtcccgctccaaggcttcaaggctttgcaaggttctacaggcgcggcagggccccggctgttcaccatccacctgatagacgcgaacacagacaaccttccgaaggcccatacctgctttaaccggatcgacattccaccatatgagtcctatgagaagctctacgagaagctgctgacagccgtggaggagacctgcgggtttgctgtggagtga

SEQ ID NO: 53 - DNA, 2247 bp, Homo sapiens
  source 1..2247: mol_type = unassigned DNA
atgtctaaccccggacgccggaggaacgggcccgtcaagctgcgcctgacagtactctgtgcaaaaaacctggtgaaaaaggattttttccgacttcctgatccatttgctaaggtggtggttgatggatctgggcaatgccattctacagatactgtgaagaatacgcttgatccaaagtggaatcagcattatgacctgtatattggaaagtctgattcagttacgatcagtgtatggaatcacaagaagatccataagaaacaaggtgctggatttctcggttgtgttcgtcttctttccaatgccatcaaccgcctcaaagacactggttatcagaggttggatttatgcaaactcgggccaaatgacaatgatacagttagaggacagatagtagtaagtcttcagtccagagaccgaataggcacaggaggacaagttgtggactgcagtcgtttatttgataacgatttaccagacggctgggaagaaaggagaaccgcctctggaagaatccagtatctaaaccatataacaagaactacgcaatgggagcgcccaacacgaccggcatccgaatattctagccctggcagacctcttagctgctttgttgatgagaacactccaattagtggaacaaatggtgcaacatgtggacagtcttcagatcccaggctggcagagaggagagtcaggtcacaacgacatagaaattacatgagcagaacacatttacatactcctccagacctaccagaaggctatgaacagaggacaacgcaacaaggccaggtgtatttcttacatacacagactggtgtgagcacatggcatgatccaagagtgcccagggatcttagcaacatcaattgtgaagagcttggtccattgcctcctggatgggagatccgtaatacggcaacaggcagagtttatttcgttgaccataacaacagaacaacacaatttacagatcctcggctgtctgctaacttgcatttagttttaaatcggcagaaccaattgaaagaccaacagcaacagcaagtggtatcgttatgtcctgatgacacagaatgcctgacagtcccaaggtacaagcgagacctggttcagaaactaaaaattttgcggcaagaactttcccaacaacagcctcaggcaggtcattgccgcattgaggtttccagggaagagatttttgaggaatcatatcgacaggtcatgaaaatgagaccaaaagatctctggaagcgattaatgataaaatttcgtggagaagaaggccttgactatggaggcgttgccagggaatggttgtatctcttgtcacatgaaatgttgaatccatactatggcctcttccagtattcaagagatgatatttatacattgcagatcaatcctgattctgcagttaatccggaacatttatcctatttccactttgttggacgaataatgggaatggctgtgtttcatggacattatattgatggtggtttcacattgcctttttataagcaattgcttgggaagtcaattaccttggatgacatggagttagtagatccggatcttcacaacagtttagtgtggatacttgagaatgatattacaggtgttttggaccataccttctgtgttgaacataatgcatatggtgaaattattcagcatgaacttaaaccaaatggcaaaagtatccctgttaatgaagaaaataaaaaagaatatgtcaggctctatgtgaactggagatttttacgaggcattgaggctcaattcttggctctgcagaaaggatttaatgaagtaattccacaacatctgctgaagacatttgatgagaaggagttagagctcattatttgtggacttggaaagatagatgttaatgactggaaggtaaacacccggttaaaacactgtacaccagacagcaacattgtcaaatggttctggaaagctgtggagttttttgatgaagagcgacgagcaagattgcttcagtttgtgacaggatcctctcgagtgcctctgcagggcttcaaagcattgcaaggtgctgcaggcccgagactctttaccatacaccagattgatgcctgcactaacaacctgccgaaagcccacacttgcttcaatcgaatagacattccaccctatgaaagctatgaaaagctatatgaaaagctgctaacagccattgaagaaacatgtggatttgctgtggaatga

SEQ ID NO: 54 - DNA, 2869 bp, Homo sapiens
  source 1..2869: mol_type = unassigned DNA
ggagtcgccgccgccccgagttccggtaccatgcatttcacggtggccttgtggagacaacgccttaacccaaggaagtgactcaaactgtgagaactccaggttttccaacctattggtggtatgtctgacagtggatcacaacttggttcaatgggtagcctcaccatgaaatcacagcttcagatcactgtcatctcagcaaaacttaaggaaaataagaagaattggtttggaccaagtccttacgtagaggtcacagtagatggacagtcaaagaagacagaaaaatgcaacaacacaaacagtcccaagtggaagcaaccccttacagttatcgttacccctgtgagtaaattacattttcgtgtgtggagtcaccagacactgaaatctgatgttttgttgggaactgctgcattagatatttatgaaacattaaagtcaaacaatatgaaacttgaagaagtagttgtgactttgcagcttggaggtgacaaagagccaacagagacaataggagacttgtcaatttgtcttgatgggctacagttagagtctgaagttgttaccaatggtgaaactacatgttcagaaagtgcttctcagaatgatgatggctccagatccaaggatgaaacaagagtgagcacaaatggatcagatgaccctgaagatgcaggagctggtgaaaataggagagtcagtgggaataattctccatcactctcaaatggtggttttaaaccttctagacctccaagaccttcacgaccaccaccacccaccccacgtagaccagcatctgtcaatggttcaccatctgccacttctgaaagtgatgggtctagtacaggctctctgccgccgacaaatacaaatacaaatacatctgaaggagcaacatctggattaataattcctcttactatatctggaggctcaggccctaggccattaaatcctgtaactcaagctcccttgccacctggttgggagcagagagtggaccagcacgggcgagtttactatgtagatcatgttgagaaaagaacaacatgggatagaccagaacctctacctcctggctgggaacggcgggttgacaacatgggacgtatttattatgttgaccatttcacaagaacaacaacgtggcagaggccaacactggaatccgtccggaactatgaacaatggcagctacagcgtagtcagcttcaaggagcaatgcagcagtttaaccagagattcatttatgggaatcaagatttatttgctacatcacaaagtaaagaatttgatcctcttggtccattgccacctggatgggagaagagaacagacagcaatggcagagtatatttcgtcaaccacaacacacgaattacacaatgggaagaccccagaagtcaaggtcaattaaatgaaaagcccttacctgaaggttgggaaatgagattcacagtggatggaattccatattttgtggaccacaatagaagaactaccacctatatagatccccgcacaggaaaatctgccctagacaatggacctcagatagcctatgttcgggacttcaaagcaaaggttcagtatttccggttctggtgtcagcaactggccatgccacagcacataaagattacagtgacaagaaaaacattgtttgaggattcctttcaacagataatgagcttcagtccccaagatctgcgaagacgtttgtgggtgatttttccaggagaagaaggtttagattatggaggtgtagcaagagaatggttctttcttttgtcacatgaagtgttgaacccaatgtattgcctgtttgaatatgcagggaaggataactactgcttgcagataaaccccgcttcttacatcaatccagatcacctgaaatattttcgttttattggcagatttattgccatggctctgttccatgggaaattcatagacacgggtttttctttaccattctataagcgtatcttgaacaaaccagttggactcaaggatttagaatctattgatccagaattttacaattctctcatctgggttaaggaaaacaatattgaggaatgtgatttggaaatgtacttctccgttgacaaagaaattctaggtgaaattaagagtcatgatctgaaacctaatggtggcaatattcttgtaacagaagaaaataaagaggaatacatcagaatggtagctgagtggaggttgtctcgaggtgttgaagaacagacacaagctttctttgaaggctttaatgaaattcttccccagcaatatttgcaatactttgatgcaaaggaattagaggtccttttatgtggaatgcaagagattgatttgaatgactggcaaagacatgccatctaccgtcattatgcaaggaccagcaaacaaatcatgtggttttggcagtttgttaaagaaattgataatgagaagagaatgagacttctgcagtttgttactggaacctgccgattgccagtaggaggatttgctgatctcatggggagcaatggaccacagaaattctgcattgaaaaagttgggaaagaaaattggctacccagaagtcatacctgttttaatcgcctggacctgccaccatacaagagctatgagcaactgaaggaaaagctgttgtttgccatagaagaaacagaaggatttggacaagagtaacttctgagaacttgcaccatgaatgggcaagaacttatttgcaatgtttgtccttctctgcctgttgcacatcttgtaaaattggacaatggctctttagagagttatctgagtgtaagtaaattaatgttctcatttaaaaaaaaaaaaaaaaaaa

SEQ ID NO: 55 - DNA, 5169 bp, Homo sapiens
  source 1..5169: mol_type = unassigned DNA
gcgcatcaggcgctgttgttggagccggaacaccgtgcgactctgaccgaaccggccccctcctcgcgcacacactcgccgagccgcgcgcgcccctccgccgtgacagtggccgtggcctccgctctctcggggcacccggcagccagagcgcagcgagagcgggcggtcgccagggtcccctccccagccagtcccaggcgcccggtgcactatgcggggcacgtgcgccccccagctctaatctgcgcgctgacaggagcatgatctgtgcccaggccagggctgccaaggaattgatgcgcgtacacgtggtgggtcattatgctgctacacctgtgtagtgtgaagaatctgtaccagaacaggtttttaggcctggccgccatggcgtctccttctagaaactcccagagccgacgccggtgcaaggagccgctccgatacagctacaaccccgaccagttccacaacatggacctcaggggcggcccccacgatggcgtcaccattccccgctccaccagcgacactgacctggtcacctcggacagccgctccacgctcatggtcagcagctcctactattccatcgggcactctcaggacctggtcatccactgggacataaaggaggaagtggacgctggggactggattggcatgtacctcattgatgaggtcttgtccgaaaactttctggactataaaaaccgtggagtcaatggttctcatcggggccagatcatctggaagatcgatgccagctcgtactttgtggaacctgaaactaagatctgcttcaaatactaccatggagtgagtggggccctgcgagcaaccacccccagtgtcacggtcaaaaactcggcagctcctatttttaaaagcattggtgctgatgagaccgtccaaggacaaggaagtcggaggctgatcagcttctctctctcagatttccaagccatggggttgaagaaagggatgtttttcaacccagacccttatctgaagatttccattcagcctgggaaacacagcatcttccccgccctccctcaccatggacaggagaggagatccaagatcataggcaacaccgtgaaccccatctggcaggccgagcaattcagttttgtgtccttgcccactgacgtgctggaaattgaggtgaaggacaagtttgccaagagccgccccatcatcaagcgcttcttgggaaagctgtcgatgcccgttcaaagactcctggagagacacgccataggggatagggtggtcagctacacacttggccgcaggcttccaacagatcatgtgagtggacagctgcaattccgatttgagatcacttcctccatccacccagatgatgaggagatttccctgagtaccgagcctgagtcagcccaaattcaggacagccccatgaacaacctgatggaaagcggcagtggggaacctcggtctgaggcaccagagtcctctgagagctggaagccagagcagctgggtgagggcagtgtccccgatggtccagggaaccaaagcatagagctttccagaccagctgaggaagcagcagtcatcacggaggcaggagaccagggcatggtctctgtgggacctgaaggggctggggagctcctggcccaggtgcaaaaggacatccagcctgcccccagtgcagaagagctggccgagcagctggacctgggtgaggaggcatcagcactgctgctggaagacggtgaagccccagccagcaccaaggaggagcccttggaggaggaagcaacgacccagagccgggctggaagggaagaagaggagaaggagcaggaggaggagggagatgtgtctaccctggagcagggagagggcaggctgcagctgcgggcctcggtgaagagaaaaagcaggccctgctccttgcctgtgtccgagctggagacggtgatcgcgtcagcctgcggggaccccgagaccccgcggacacactacatccgcatccacaccctgctgcacagcatgccctccgcccagggcggcagcgcggcagaggaggaggacggcgcggaggaggagtccaccctcaaggactcctcggagaaggatgggctcagcgaggtggacacggtggccgctgacccgtctgccctggaagaggacagagaagagcccgagggggctactccaggcacggcgcaccctggccactccgggggccacttccccagcctggccaatggcgcggcccaggatggcgacacgcaccccagcaccgggagcgagagcgactccagccccaggcaaggcggggaccacagttgcgagggctgtgacgcgtcctgctgcagcccctcgtgctacagctcctcgtgctacagcacgtcctgctacagcagctcgtgctacagcgcctcgtgctacagcccctcctgctacaacggcaacaggttcgccagccacacgcgcttctcctccgtggacagcgccaagatctccgagagcacggtcttctcctcgcaagacgacgaggaggaggagaacagcgcgttcgagtcggtacccgactccatgcagagccctgagctggacccggagtccacgaacggcgctgggccgtggcaagacgagctggccgcccctagcgggcacgtggaaagaagcccggaaggtctggaatcccccgtggcaggtccaagcaatcggagagaagactgggaagctcgaattgacagccacgggcgggtcttttatgtggaccacgtgaaccgcacaaccacctggcagcgtccgacggcagcagccaccccggatggcatgcggagatcggggtccatccagcagatggagcaactcaacaggcggtatcaaaacattcagcgaaccattgcaacagagaggtccgaagaagattctggcagccaaagctgcgagcaagccccagcaggaggaggcggaggtggagggagtgactcagaagccgaatcttcccagtccagcttagatctaaggagagaggggtcactttctccagtgaactcacaaaaaatcaccttgctgctgcagtccccagcggtcaagttcatcaccaaccccgagttcttcactgtgctacacgccaattatagtgcctaccgagtcttcaccagtagcacctgcttaaagcacatgattctgaaagtccgacgggatgctcgcaattttgaacgctaccagcacaaccgggacttggtgaatttcatcaacatgttcgcagacactcggctggaactgccccggggctgggagatcaaaacggaccagcagggaaagtcttttttcgtggaccacaacagtcgagctaccactttcattgacccccgaatccctcttcagaacggtcgtcttcccaatcatctaactcaccgacagcacctccagaggctccgaagttacagcgccggagaggcctcagaagtttctagaaacagaggagcctctttactggccaggccaggacacagcttagtagctgctattcgaagccaacatcaacatgagtcattgccactggcatataatgacaagattgtggcatttcttcgccagccaaacatttttgaaatgctgcaagagcgtcagccaagcttagcaagaaaccacacactcagggagaaaatccattacattcggactgagggtaatcacgggcttgagaagttgtcctgtgatgcggatctggtcattttgctgagtctctttgaagaagagattatgtcctacgtccccctgcaggctgccttccaccctgggtatagcttctctccccgatgttcaccctgttcttcacctcagaactccccaggtttacagagagccagtgcaagagccccttccccctaccgaagagactttgaggccaagctccgcaatttctacagaaaactggaagccaaaggatttggtcagggtccggggaaaattaagctcattattcgccgggatcatttgttggagggaaccttcaatcaggtgatggcctattcgcggaaagagctccagcgaaacaagctctacgtcacctttgttggagaggagggcctggactacagtggcccctcgcgggagttcttcttccttctgtctcaggagctcttcaacccttactatggactctttgagtactcggcaaatgatacttacacggtgcagatcagccccatgtccgcatttgtagaaaaccatcttgagtggttcaggtttagcggtcgcatcctgggtctggctctgatccatcagtaccttcttgacgctttcttcacgaggcccttctacaaggcactcctgagactgccctgtgatttgagtgacctggaatatttggatgaggaattccaccagagtttgcagtggatgaaggacaacaacatcacagacatcttagacctcactttcactgttaatgaagaggtttttggacaggtcacggaaagggagttgaagtctggaggagccaacacacaggtgacggagaaaaacaagaaggagtacatcgagcgcatggtgaagtggcgggtggagcgcggcgtggtacagcagaccgaggcgctggtgcgcggcttctacgaggttgtagactcgaggctggtgtccgtgtttgatgccagggagctggagctggtgatagctggcaccgcggaaatcgacctaaatgactggcggaataacactgagtaccggggaggttaccacgatgggcatcttgtgatccgctggttctgggctgcggtggagcgcttcaataatgagcagaggctgagattactgcagtttgtcacgggaacatccagcgtgccctacgaaggcttcgcagccctccgtgggagcaatgggcttcggcgcttctgcatagagaaatgggggaaaattacttctctccccagggcacacacatgcttcaaccgactggatcttccaccgtatccctcgtactccatgttgtatgaaaagctgttaacagcagtagaggaaaccagcacctttggacttgagtgaggacatggaacctcgcctgacattttcctggccagtgacatcacccttcctgggatgatccccttttccctttcccttaatcaactctcctttgattttggtattccatgatttttattttcaaac

SEQ ID NO: 56 - DNA, 2715 bp, Homo sapiens
  source 1..2715: mol_type = unassigned DNA
agagttccatcagagcctgcagtggatgaaagacaatgatatccatgacatcctagacctcacgttcactgtgaacgaagaagtatttgggcagataactgaacgagaattaaagccagggggtgccaatatcccagttacagagaagaacaagaaggagtacatcgagaggatggtgaagtggaggattgagaggggtgttgtacagcaaacagagagcttagtgcgtggcttctatgaggtggtggatgccaggctggtatctgtttttgatgcaagagaactggaattggtcatcgcaggcacagctgaaatagacctaagtgattggagaaacaacacagaatatagaggaggataccatgacaatcatattgtaattcggtggttctgggctgcagtggaaagattcaacaatgaacaacgactaaggttgttacagtttgttacaggcacatccagcattccctatgaaggatttgcttcactccgagggagtaacggcccaagaagattctgtgtggagaaatgggggaaaatcactgctcttcccagagcgcatacatgttttaaccgtctggatctgcctccctacccatccttttccatgctttatgaaaaactgttgacagcagttgaagaaaccagtacttttggacttgagtgacctggaagctgaatgcccatctctgtggacaggcagtttcagaagctgccttctagaagaatgattgaacattggaagtttcaagaggatgcttcctttaggataaagctacgtgctgttgttttccaggaacaagtgctctgtcacatttggggactggagatgagtcctcttggaaggatttgggtgagcttgatgcccagggaacaacccaaccgtctttcaatcaacagttcttgactgccaaactttttccatttgttatgttccaagacaaagatgaacccatacatgatcagctccacggtaatttttagggactcaggagaatcttgaaacttacccttgaacgtggttcaagccaaactggcagcatttggcccaatctccaaattagagcaagttaaataatataataaaagtaaatatatttcctgaaagtacattcatttaagccctaagttataacagaatattcatttcttgcttatgagtgcctgcatggtgtgcaccataggtttccgctttcatgggacatgagtgaaaatgaaaccaagtcaatatgaggtacctttacagatttgcaataagatggtctgtgacaatgtatatgcaagtggtatgtgtgtaattatggctaaagacaaaccattattcagtgaattactaatgacagattttatgctttataatgcatgaaaacaattttaaaataactagcaattaatcacagcatatcaggaaaaagtacacagtgagttctgtttattttttgtaggctcattatgtttatgttctttaagatgtatataagaacctacttatcatgctgtatgtatcactcattccattttcatgttccatgcatactcgggcatcatgctaatatgtatccttttaagcactctcaaggaaacaaaagggccttttatttttataaaggtaaaaaaaattccccaaatattttgcactgaatgtaccaaaggtgaagggacattacaatatgactaacagcaactccatcacttgagaagtataatagaaaatagcttctaaatcaaacttccttcacagtgccgtgtctaccactacaaggactgtgcatctaagtaataattttttaagattcactatatgtgatagtatgatatgcatttatttaaaatgcattagactctcttccatccatcaaatactttacaggatggcatttaatacagatatttcgtatttcccccactgctttttatttgtacagcatcattaaacactaagctcagttaaggagccatcagcaacactgaagagatcagtagtaagaattccattttccctcatcagtgaagacaccacaaattgaaactcagaactatatttctaagcctgcattttcactgatgcataattttcttattaatattaagagacagtttttctatggcatctccaaaactgcatgacatcactagtcttacttctgcttaattttatgagaaggtattcttcattttaattgcttttgggattactccacatctttgtttatttcttgactaatcagattttcaatagagtgaagttaaattgggggtcataaaagcattggattgacatatggtttgccagcctatgggtttacaggcattgcccaaacatttctttgagatctatatttataagcagccatggaattcctattatgggatgttggcaatcttacattttatagaggtcatatgcatagttttcataggtgttttgtaagaactgattgctctcctgtgagttaagctatgtttactactgggaccctcaagaggaataccacttatgttacactcctgcactaaaggcacgtactgcagtgtgaagaaatgttctgaaaaagggttatagaaatctggaaataagaaaggaagagctctctgtattctataattggaagagaaaaaaagaaaaacttttaactggaaatgttagtttgtacttattgatcatgaatacaagtatatatttaattttgcaaaaaaaaaaaaaaaaaaaaaaag

SEQ ID NO: 57 - DNA, 2403 bp, synthetic construct
  source 1..2403: mol_type = other DNA
atggggcgagtgcagctcttcgagatcagcctgagccacggccgcgtcgtctacagccccggggagccgttggctgggaccgtgcgcgtgcgcctgggggcaccgctgccgttccgagccatccgggtgacctgcataggttcctgcggggtctccaacaaggctaatgacacagcgtgggtagtggaggagggttacttcaacagttccctgtcgctggcagacaaggggagcctgcccgctggagagcacagcttccccttccagttcctgcttcctgccactgcacccacgtcctttgagggtcctttcgggaagatcgtgcaccaggtgagggccgccatccacacgccacggttttccaaggatcacaagtgcagcctcgtgttctatatcttgagccccttgaacctgaacagcatcccagacattgagcaacccaacgtggcctctgccaccaagaagttctcctacaagctggtgaagacgggcagcgtggtcctcacagccagcactgatctccgcggctatgtggtggggcaggcactgcagctgcatgccgacgttgagaaccagtcaggcaaggacaccagccctgtggtggccagtctgctgcagaaagtgtcctataaggccaagcgctggatccacgacgtacggaccattgcggaggtggagggtgcgggcgtcaaggcctggcggcgggcgcagtggcacgagcagatcctggtgcctgccttgccccagtcggccctgccgggctgcagcctcatccacatcgactactacttacaggtctctctgaaggcgccggaagctactgtgaccctcccggtcttcattggcaatattgctgtgaaccatgccccagtgagcccccggccaggcctggggctgcctcctggggccccacccctggtggtgccttccgcaccaccccaggaggaggctgaggctgaggctgcggctggcggcccccacttcttggaccccgtcttcctctccaccaagagccattcgcagcggcagcccctgctggccaccttgagttctgtgcctggtgcgccggagccctgccctcaggatggcagccctgcctcacacccgctgcaccctcccttgtgcatttcaacaggtgccactgtcccctactttgcagagggctccggggggccagtgcccactaccagcaccttgattcttcctccagagtacagttcttggggctacccctatgaggccccaccgtcttatgagcagagctgcggcggcgtggaacccagcctgacccctgagagctctggtggcggaggctcgggcggaggtgggtcgggtggcggcggatcaatgggcccaaagaagaagagaaaggtttcgaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 58 - protein, 1536 aa, synthetic construct
ATGAAGTGCCTTTTGTACTTAGCCTTTTTATTCATTGGGGTGAATTGCAAGTTCACCATAGTTTTTCCACACAACCAAAAAGGAAACTGGAAAAATGTTCCTTCTAATTACCATTATTGCCCGTCAAGCTCAGATTTAAATTGGCATAATGACTTAATAGGCACAGCCTTACAAGTCAAAATGCCCAAGAGTCACAAGGCTATTCAAGCAGACGGTTGGATGTGTCATGCTTCCAAATGGGTCACTACTTGTGATTTCCGCTGGTATGGACCGAAGTATATAACACATTCCATCCGATCCTTCACTCCATCTGTAGAACAATGCAAGGAAAGCATTGAACAAACGAAACAAGGAACTTGGCTGAATCCAGGCTTCCCTCCTCAAAGTTGTGGATATGCAACTGTGACGGATGCCGAAGCAGTGATTGTCCAGGTGACTCCTCACCATGTGCTGGTTGATGAATACACAGGAGAATGGGTTGATTCACAGTTCATCAACGGAAAATGCAGCAATTACATATGCCCCACTGTCCATAACTCTACAACCTGGCATTCTGACTATAAGGTCAAAGGGCTATGTGATTCTAACCTCATTTCCATGGACATCACCTTCTTCTCAGAGGACGGAGAGCTATCATCCCTGGGAAAGGAGGGCACAGGGTTCAGAAGTAACTACTTTGCTTATGAAACTGGAGGCAAGGCCTGCAAAATGCAATACTGCAAGCATTGGGGAGTCAGACTCCCATCAGGTGTCTGGTTCGAGATGGCTGATAAGGATCTCTTTGCTGCAGCCAGATTCCCTGAATGCCCAGAAGGGTCAAGTATCTCTGCTCCATCTCAGACCTCAGTGGATGTAAGTCTAATTCAGGACGTTGAGAGGATCTTGGATTATTCCCTCTGCCAAGAAACCTGGAGCAAAATCAGAGCGGGTCTTCCAATCTCTCCAGTGGATCTCAGCTATCTTGCTCCTAAAAACCCAGGAACCGGTCCTGCTTTCACCATAATCAATGGTACCCTAAAATACTTTGAGACCAGATACATCAGAGTCGATATTGCTGCTCCAATCCTCTCAAGAATGGTCGGAATGATCAGTGGAACTACCACAGAAAGGGAACTGTGGGATGACTGGGCACCATATGAAGACGTGGAAATTGGACCCAATGGAGTTCTGAGGACCAGTTCAGGATATAAGTTTCCTTTATACATGATTGGACATGGTATGTTGGACTCCGATCTTCATCTTAGCTCAAAGGCTCAGGTGTTCGAACATCCTCACATTCAAGACGCTGCTTCGCAACTTCCTGATGATGAGAGTTTATTTTTTGGTGATACTGGGCTATCCAAAAATCCAATCGAGCTTGTAGAAGGTTGGTTCAGTAGTTGGAAAAGCTCTATTGCCTCTTTTTTCTTTATCATAGGGTTAATCATTGGACTATTCTTGGTTCTCCGAGTTGGTATCCATCTTTGCATTAAATTAAAGCACACCAAGAAAAGACAGATTTATACAGACATAGAGATGAACCGACTTGGAAAGTAA

SEQ ID NO: 59 - DNA, 2034 bp, synthetic construct
  source 1..2034: mol_type = other DNA
atggggcgagtgcagctcttcgagatcagcctgagccacggccgcgtcgtctacagccccggggagccgttggctgggaccgtgcgcgtgcgcctgggggcaccgctgccgttccgagccatccgggtgacctgcataggttcctgcggggtctccaacaaggctaatgacacagcgtgggtagtggaggagggttacttcaacagttccctgtcgctggcagacaaggggagcctgcccgctggagagcacagcttccccttccagttcctgcttcctgccactgcacccacgtcctttgagggtcctttcgggaagatcgtgcaccaggtgagggccgccatccacacgccacggttttccaaggatcacaagtgcagcctcgtgttctatatcttgagccccttgaacctgaacagcatcccagacattgagcaacccaacgtggcctctgccaccaagaagttctcctacaagctggtgaagacgggcagcgtggtcctcacagccagcactgatctccgcggctatgtggtggggcaggcactgcagctgcatgccgacgttgagaaccagtcaggcaaggacaccagccctgtggtggccagtctgctgcagaaagtgtcctataaggccaagcgctggatccacgacgtacggaccattgcggaggtggagggtgcgggcgtcaaggcctggcggcgggcgcagtggcacgagcagatcctggtgcctgccttgccccagtcggccctgccgggctgcagcctcatccacatcgactactacttacaggtctctctgaaggcgccggaagctactgtgaccctcccggtcttcattggcaatattgctgtgaaccatgccccagtgagcccccggccaggcctggggctgcctcctggggccccacccctggtggtgccttccgcaccaccccaggaggaggctgaggctgaggctgcggctggcggcccccacttcttggaccccgtcttcctctccaccaagagccattcgcagcggcagcccctgctggccaccttgagttctgtgcctggtgcgccggagccctgccctcaggatggcagccctgcctcacacccgctgcaccctcccttgtgcatttcaacaggtgccactgtcccctactttgcagagggctccggggggccagtgcccactaccagcaccttgattcttcctccagagtacagttcttggggctacccctatgaggccccaccgtcttatgagcagagctgcggcggcgtggaacccagcctgacccctgagagcggcggcagcagcggcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 60 - protein, 677 aa, synthetic construct
MGRVQLFEISLSHGRVVYSPGEPLAGTVRVRLGAPLPFRAIRVTCIGSCGVSNKANDTAWVVEEGYFNSSLSLADKGSLPAGEHSFPFQFLLPATAPTSFEGPFGKIVHQVRAAIHTPRFSKDHKCSLVFYILSPLNLNSIPDIEQPNVASATKKFSYKLVKTGSVVLTASTDLRGYVVGQALQLHADVENQSGKDTSPVVASLLQKVSYKAKRWIHDVRTIAEVEGAGVKAWRRAQWHEQILVPALPQSALPGCSLIHIDYYLQVSLKAPEATVTLPVFIGNIAVNHAPVSPRPGLGLPPGAPPLVVPSAPPQEEAEAEAAAGGPHFLDPVFLSTKSHSQRQPLLATLSSVPGAPEPCPQDGSPASHPLHPPLCISTGATVPYFAEGSGGPVPTTSTLILPPEYSSWGYPYEAPPSYEQSCGGVEPSLTPESGGSSGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 61 - DNA, 2025 bp, synthetic construct
  source 1..2025: mol_type = other DNA
atggggcgagtgcagctcttcgagatcagcctgagccacggccgcgtcgtctacagccccggggagccgttggctgggaccgtgcgcgtgcgcctgggggcaccgctgccgttccgagccatccgggtgacctgcataggttcctgcggggtctccaacaaggctaatgacacagcgtgggtagtggaggagggttacttcaacagttccctgtcgctggcagacaaggggagcctgcccgctggagagcacagcttccccttccagttcctgcttcctgccactgcacccacgtcctttgagggtcctttcgggaagatcgtgcaccaggtgagggccgccatccacacgccacggttttccaaggatcacaagtgcagcctcgtgttctatatcttgagccccttgaacctgaacagcatcccagacattgagcaacccaacgtggcctctgccaccaagaagttctcctacaagctggtgaagacgggcagcgtggtcctcacagccagcactgatctccgcggctatgtggtggggcaggcactgcagctgcatgccgacgttgagaaccagtcaggcaaggacaccagccctgtggtggccagtctgctgcagaaagtgtcctataaggccaagcgctggatccacgacgtacggaccattgcggaggtggagggtgcgggcgtcaaggcctggcggcgggcgcagtggcacgagcagatcctggtgcctgccttgccccagtcggccctgccgggctgcagcctcatccacatcgactactacttacaggtctctctgaaggcgccggaagctactgtgaccctcccggtcttcattggcaatattgctgtgaaccatgccccagtgagcccccggccaggcctggggctgcctcctggggccccacccctggtggtgccttccgcaccaccccaggaggaggctgaggctgaggctgcggctggcggcccccacttcttggaccccgtcttcctctccaccaagagccattcgcagcggcagcccctgctggccaccttgagttctgtgcctggtgcgccggagccctgccctcaggatggcagccctgcctcacacccgctgcaccctcccttgtgcatttcaacaggtgccactgtcccctactttgcagagggctccggggggccagtgcccactaccagcaccttgattcttcctccagagtacagttcttggggctacccctatgaggccccaccgtcttatgagcagagctgcggcggcgtggaacccagcctgacccctgagagcggcggcagcagcggcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 62 - protein, 674 aa, synthetic construct
MGRVQLFEISLSHGRVVYSPGEPLAGTVRVRLGAPLPFRAIRVTCIGSCGVSNKANDTAWVVEEGYFNSSLSLADKGSLPAGEHSFPFQFLLPATAPTSFEGPFGKIVHQVRAAIHTPRFSKDHKCSLVFYILSPLNLNSIPDIEQPNVASATKKFSYKLVKTGSVVLTASTDLRGYVVGQALQLHADVENQSGKDTSPVVASLLQKVSYKAKRWIHDVRTIAEVEGAGVKAWRRAQWHEQILVPALPQSALPGCSLIHIDYYLQVSLKAPEATVTLPVFIGNIAVNHAPVSPRPGLGLPPGAPPLVVPSAPPQEEAEAEAAAGGPHFLDPVFLSTKSHSQRQPLLATLSSVPGAPEPCPQDGSPASHPLHPPLCISTGATVPYFAEGSGGPVPTTSTLILPPEYSSWGYPYEAPPSYEQSCGGVEPSLTPESGGSSGMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 63 - DNA, 4104 bp, Streptococcus pyogenes
  source 1..4104: mol_type = unassigned DNA
atggataagaaatactcaataggcttagatatcggcacaaatagcgtcggatgggcggtgatcactgatgattataaggttccgtctaaaaagttcaaggttctgggaaatacagaccgccacagtatcaaaaaaaatcttataggggctcttttatttggcagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggcagattctactgataaagcggatttgcgcttaatctatttggccttagcgcatatgattaagtttcgtggtcattttttgattgagggagatttaaatcctgataatagtgatgtggacaaactatttatccagttggtacaaatctacaatcaattatttgaagaaaaccctattaacgcaagtagagtagatgctaaagcgattctttctgcacgattgagtaaatcaagacgattagaaaatctcattgctcagctccccggtgagaagagaaatggcttgtttgggaatctcattgctttgtcattgggattgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatagtgaaataactaaggctcccctatcagcttcaatgattaagcgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgagggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggcgcctaccatgatttgctaaaaattattaaagataaagattttttggataatgaagaaaatgaagatatcttagaggatattgttttaacattgaccttatttgaagatagggggatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagatattcaaaaagcacaggtgtctggacaaggccatagtttacatgaacagattgctaacttagctggcagtcctgctattaaaaaaggtattttacagactgtaaaaattgttgatgaactggtcaaagtaatggggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctacaaaatggaagagacatgtatgtggaccaagaattagatattaatcgtttaagtgattatgatgtcgatcacattgttccacaaagtttcattaaagacgattcaatagacaataaggtactaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 64 - protein, 1367 aa, Streptococcus pyogenes
MDKKYSIGLDIGTNSVGWAVITDDYKVPSKKFKVLGNTDRHSIKKNLIGALLFGSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQIYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGAYHDLLKIIKDKDFLDNEENEDILEDIVLTLTLFEDRGMIEERLKTYAHLFDDKVMKQLKRRRYTGWGRLSRKLINGIRDKQSGKTILDFLKSDGFANRNFMQLIHDDSLTFKEDIQKAQVSGQGHSLHEQIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 65 - DNA, 4212 bp, Streptococcus pyogenes
  source 1..4212: mol_type = unassigned DNA
atggataaaaagtattctattggtttagacatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggatcccccaagaagaagaggaaagtctcgagcgactacaaagaccatgacggtgattataaagatcatgacatcgattacaaggatgacgatgacaaggctgcagga

SEQ ID NO: 66 - protein, 1368 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 67 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

(19) United States
(12) Patent Application Publication
(10) Pub. No.: US 2026/0102508 A1
(43) Pub. Date: Apr. 16, 2026

(54) ARRDC1-Mediated Microvesicle-Based Delivery of Therapeutic Agents to Cells and Tissues of the Eye

(71) Applicant: Turn Biotechnologies, Inc., Mountain View, CA (US)

(72) Inventor: Yashodhan Chinchore, Newton, MA (US)

(21) Appl. No.: 19/116,655

(22) PCT Filed: Sep. 29, 2023
(86) PCT No.: PCT/US2023/034197

§ 371 Date: Mar. 28, 2025

Related U.S. Application Data
(60) Provisional application No. 63/412,161, filed on Sep. 30, 2022.
(60) Provisional application No. 63/533,841, filed on Aug. 21, 2023.
(60) Provisional application No. 63/538,152, filed on Sep. 13, 2023.

(51) Int. Cl. A61K 47/69; A61K 47/64

(52) U.S. Cl. CPC A61K 47/6901; A61K 47/64

(57) ABSTRACT

The present invention provides methods, systems, and compositions for ARMM-mediated delivery of molecules of interest (e.g., therapeutic agents) to cells and tissues of the eye. The present invention further relates to compositions and methods of producing, testing, and administering ARRDC1-mediated microvesicles (“ARMMs”) to internal structures of the eye. More particularly, the present invention provides compositions and methods of producing, testing, and administering ARMMs particles comprising one or more therapeutic agents (e.g., biological molecules including, but not limited to, CRISPR/Cas9 and other similar endonucleases, base editors, small molecules, proteins, and nucleic acids (e.g., DNA, RNA, siRNA, mRNA, miRNA, and the like)). Also provided are methods of administering therapeutic agents associated with ARMMs, including, but not limited to, methods of treating or contacting cells and tissues of the eye in one or more dosing regimens. In particular, the present invention provides methods of administering therapeutic agents via ARMMs to the cells and tissues that comprise the retina or into the subretinal space. Additionally, the present invention relates to methods of manufacturing (e.g., culturing, clarifying, separating, and concentrating) the inventive compositions from stable producer cell lines and from cell cultures.

### CLAIM OF PRIORITY

This application claims the benefit of U.S. Provisional Application Ser. No. 63/412,161, filed on Sep. 30, 2022, 63/533,841, filed on Aug. 21, 2023, and 63/538,152, filed on Sep. 13, 2023. The entire contents of the foregoing are incorporated herein by reference.

### FIELD OF THE INVENTION

The present invention provides methods, systems, and compositions for ARMM-mediated delivery of molecules of interest (e.g., therapeutic agents) to cells and tissues of the eye. The present invention further relates to compositions and methods of producing, testing, and administering ARRDC1-mediated microvesicles (“ARMMs”) to internal structures of the eye. More particularly, the present invention provides compositions and methods of producing, testing, and administering ARMMs particles comprising one or more therapeutic agents (e.g., biological molecules including, but not limited to, CRISPR/Cas9 and other similar endonucleases, base editors, small molecules, proteins, and nucleic acids (e.g., DNA. RNA, siRNA, mRNA, miRNA, and the like)). Also provided are methods of administering therapeutic agents associated with ARMMs, including, but not limited to, methods of treating or contacting cells and tissues of the eye in one or more dosing regimens. In particular, the present invention provides methods of administering therapeutic agents via ARMMs to the cells and tissues that comprise the retina or into the subretinal space. Additionally, the present invention relates to methods of manufacturing (e.g., culturing, clarifying, separating, and concentrating) the inventive compositions from stable producer cell lines and from cell cultures.

### BACKGROUND OF THE INVENTION

Vision is unarguably an important human sense. It is perhaps the most important of the human senses based on the portion of the brain devoted to processing visual information. Against this backdrop, it is important to consider that approximately 12 million people 40 years and over in the United States have vision impairment, including about one million who are blind, three million who have vision impairment after correction, and eight million who have vision impairment due to uncorrected refractive error. (Flaxman A. D., et al., “Prevalence of visual acuity loss or blindness in the US,” JAMA Ophthalmology, 139(7):717-723 (2021)). More particularly, statistics from 2012 in the United States, indicate that about 4.2 million people aged 40 years and older suffer from presently uncorrectable vision impairment (i.e., best-corrected visual acuity in the better seeing-eye ≤20/40), out of which 1.02 million are blind (i.e., best-corrected visual acuity in the better seeing-eye ≤20/200). The number of people predicted to suffer from blindness will more than double by 2050 to approximately 8.96 million due to the increasing prevalence of diabetes and other chronic diseases and a rapidly aging population.

Additionally, approximately 6.8% of children younger than 18 years in the United States have a diagnosed eye and vision condition. Nearly three percent of U.S. children younger than 18 years are blind or visually impaired, defined as having trouble seeing even when wearing glasses or contact lenses. Vision disability in the United States is one of the top 10 disabilities among adults 18 years and older and one of the most prevalent disabling conditions among children.

More than 70% of survey respondents from the U.S. National Eye Health Education Program (“NEHEP”) 2005 Public Knowledge. Attitudes, and Practices Survey said that loss of their eyesight would have the greatest impact on their day-to-day life; however, less than 11% of respondents were aware that there are no widely accepted early warning signs of glaucoma and diabetic retinopathy.

Vision loss causes a substantial social and economic toll for millions of people including significant suffering, disability, loss of productivity, and diminished quality of life. The annual economic impact of major vision problems among the adult population 40 years and older is estimated to be more than $145 billion.

While the causes of vision-related diseases and blindness are numerous and varied, many ophthalmic diseases are presently known or suspected to result from one or more genetic abnormalities. Research indicates that roughly ten percent of the 10,000 purported human genetic disorders appear to have ocular manifestations. Genetic ocular diseases can be transmitted according to various modes of transmission, including autosomal dominant, autosomal recessive, X-linked dominant (rarely) or recessive, multifactorial inheritance, and cytoplasmic inheritance. Research into the molecular basis of ophthalmic diseases has increased in recent years, and consequently, many important discoveries in molecular ophthalmology and the genetic basis of many ophthalmological disorders have been characterized. Research into retinal diseases has revealed many genes and signal transduction pathways as promising targets for gene therapy or other therapeutics.

Many of these discoveries have contributed to more general advancements in molecular genetics as well as the art's understanding of the molecular mechanism of certain non-ophthalmological diseases. For example, the identification and cloning of the retinoblastoma gene (RB1) led to the discovery of tumor-suppressor genes. In another example, research into the cause of retinitis pigmentosa led to the identification of point mutations that encode altered photoreceptor proteins that degrade the retina, and which provided promising targets for future therapies.

Additionally, certain of these discoveries have been incorporated into gene therapy efforts directed at ameliorating ocular diseases. For instance, in gene supplementation, the so-called classical gene therapy technique, a functional copy of a gene is delivered to a target cell, usually by a non-integrating adeno-associated virus (“AAV”) vector, to supply the missing gene or restore defective gene function. Ideally, gene supplementation strategies are preferably employed to counteract autosomal recessive and X-linked forms of genetic diseases (e.g., certain types of inherited retinal dystrophies (“IRDs”)).

Briefly, IRDs can result from one or more of a wide variety of genetic mutations. Indeed, there are some 250 known genetic mutations that contribute to the various types of IRDs. The genes responsible for IRDs are mainly expressed in photoreceptor cells, and to a lesser extent, in retinal pigment epithelial cells (“RPEs”). Many IRDs result from the dysregulation of genes that encode structural proteins. The expression of these genes must be carefully regulated to maintain a proper ratio with the other cellular components to restore function. Designing successful gene supplementation compositions and methods thus requires careful consideration of the intended expression in the target cells.

In the only existing successful application of AAV-mediated gene supplementation to date, the agent voretigene neparvovec-rzyl has been approved in the United States and Europe for the treatment of Leber Congenital Amaurosis type 2 (“LCA2”). LCA2 is considered the most severe type of IRD.

Despite the success of this AAV-mediated gene supplementation therapy in ameliorating LCA2 disease, the general use of gene supplementation for other types of IRDs and other genetic retinal disorders remains a challenge. One significant challenge concerns the effectiveness of gene supplementation strategies for counteracting gain-of-function mutations in autosomal dominant IRDs compared to loss-of-function associated diseases (e.g., LCA2) since in gain-of-function mutations, the pathogenic mutant protein continues to be expressed.

Furthermore, the limited packaging capacity of the AAV-mediated vectors (˜4.5 kb) can constrain gene supplementation therapies that seek to employ these vectors. Others are exploring alternative packaging systems such as lentiviruses, non-viral vectors (e.g., plasmids), DNA nanoparticles, delivery of antisense oligonucleotides, and nucleic acid sequences for gene supplementation.

Despite advances in understanding the molecular underpinnings of many ocular diseases, there remains a need in the art for additional therapeutic agents and accompanying delivery platforms for treating diseases of the eye, in particular, diseases of the retina, and more particularly, diseases affecting the ganglion cell layer, photoreceptors, and retinal pigment epithelium (“RPE”) thereof. Nevertheless, pursuing treatments for ocular diseases can be challenging considering the unique anatomical structures and natural defense mechanisms of the eye (e.g., lack of efferent lymphatics, incomplete ocular immune privilege, tight blood-ocular barriers in retinal cells, and the like).

### SUMMARY OF THE INVENTION

This invention relates to the discovery that molecules, such as proteins and nucleic acids, including ribonucleic acids (RNAs), as well as small molecules, can be loaded into microvesicles, specifically ARRDC1-mediated microvesicles (ARMMs), for delivery to the eye, specifically cells of the retina, and more specifically, to ganglion cells, and retinal pigment epithelium cells (“RPEs”).

In certain embodiments, the ARMMs can incorporate viral envelope proteins to allow for the delivery of molecules to the retina. For example, vesicular stomatitis virus G protein (VSV-G) or rabies virus glycoprotein (RVG) can be co-expressed in, and appear on, the surface of ARMMS to target cells of the retina (e.g., RPE cells). These proteins normally function to aid viral attachment and entry of viruses into cells. For example, VSV-G mediates viral attachment to LDL receptors (“LDLR”) or LDLR family members, and RVG is known to use the nicotinic acetylcholine receptor and the low-affinity nerve growth factor receptor for viral entry. It has been found that these proteins can also aid ARMMs to attach to cells, including cells of the retina.

In addition, the ARMM delivery systems, described herein, address many limitations of current delivery systems that prevent the safe and efficient delivery of proteins and nucleic acids (e.g., RNAs (including both RNA coding for proteins and non-coding RNA) to the retina. As ARMMs are derived from an endogenous budding pathway, they are unlikely to elicit a strong immune response, unlike viral delivery systems, which are known to trigger inflammatory responses. (See, Sen et al., “Cellular unfolded protein response against viruses used in gene therapy,” Front Microbiology, 5:250, 1-16 (2014)). Additionally, ARMMs allow for the specific packaging of many types and classes of potentially therapeutic molecules (e.g., biological molecules, such as a protein or nucleic acid (e.g., DNA plasmid, mRNA, miRNA, or shRNA), or small molecules). While the present invention is not intended to be limited to any particular mechanism(s), it is contemplated, in certain embodiments, ARMMs can be delivered by fusion with or uptake by, specific recipient cells and tissues by incorporating antibodies or other types of targeting or tropism determinant molecules into or onto the ARMMs so as to recognize tissue-specific markers.

ARMMs are microvesicles that are distinct from exosomes which, like budding viruses, are produced by direct plasma membrane budding (“DPMB”). DPMB is driven by a specific interaction of TSG101 with a tetrapeptide PSAP (SEQ ID NO: 1) motif of the arrestin-domain-containing protein ARRDC1 accessory protein, which is localized to the plasma membrane through its arrestin domain. ARMMs have been described in detail, for example, in PCT application number PCT/US2013/024839, filed Feb. 6, 2013 (published as WO 2013/119602 A1 on Aug. 15, 2013) by Lu, Q., el al., and entitled “Arrdc1-Mediated Microvesicles (ARAMs) and Uses Thereof,” as well as in U.S. Pat. Nos. 9,737,480; 9,816,080; 10,260,055; and PCT Publication WO2018/067546; the entire contents of which are hereby incorporated by reference in their entirety. The ARRDC1/TSG101 interaction results in the relocation of TSG101 from endosomes to the plasma membrane and mediates the release of microvesicles that contain TSG101, ARRDC1, and other cellular components as well as the molecule of interest.

Molecules of interest, whether naturally or non-naturally, occurring include, but are not limited to, proteins, nucleic acids, and small molecules that can preferably associate with one or more ARMM related proteins (e.g., ARRDC1), or can be modified to associate with more specifically. TSG101 or ARRDC1 or specific motif(s) therein. These associations facilitate the incorporation of the molecules into ARMMs, which in turn can be used to deliver the desired payload (molecule of interest) into a targeted cell. By way of example, but not limitation, a payload RNA can be fused to a trans-activation response (TAR) element, thereby allowing it to associate with an ARRDC1 protein that is fused to an RNA binding protein, such as a Tat protein (e.g., bovine TAT protein). Alternatively, a payload protein can be fused to one or more WW domains, which associate with the PPXY (SEQ ID NO: 2) motif of ARRDC1. The association of the molecule to an ARMM-related protein (e.g., ARRDC1), facilitates the loading of the molecule into the ARRDC1-containing ARMM. Alternatively, the molecule can be fused to an ARMM protein (e.g., TSG101 or ARRDC1) to load the payload into the ARMM. The molecule can be fused to the ARMM protein (e.g., TSG101 or ARRDC1) via a linker that may be cleaved upon delivery to a target cell.

As an example, the delivery platform of ARMMs will enable multiple cis-acting structural elements of mRNAs to perform in the context of intracellular and secreted therapeutics for target cells, where these structural elements include, but are not limited to: (i) 5′ cap structure; (ii) 5′ untranslated region (UTR); (iii) the codon optimized coding sequence; (iv) 3′ UTR; (v) a 3′ poly-A tail consisting of a stretch of repeated adenine nucleotides; and (vi) inclusion of cis-acting zip code elements within RNA transcripts that are recognized by specific RNA binding proteins to cause specific cellular localization (e.g., to synapse of neurons). (See, e.g., Chin A., Lécuyer E., “RNA localization: Making its way to the center stage,” Biochim. Biophys. Acta. Gen Subj., 1861(11 Pt B):2956-2970 (2017)).

As another example, the delivery platform for ARMMs will enable multiple classes of protein and mRNA-based therapeutics to be directed to target cells (e.g., cells of the retina). Suitable therapeutic agents for use in the compositions and methods of the present invention include, but are not limited to, transmembrane proteins, cytoplasmic proteins, nuclear proteins, mitochondrial proteins, endoplasmic reticulum proteins, Golgi proteins, peroxisome proteins, lysosome proteins, and secreted proteins.

Also contemplated herein in the context of therapeutics for vision-related disorders are the targeted expression of single-chain variable fragment (scFv) antibodies composed of a fusion protein of the variable regions of the heavy (VH) and light chains (VL) of immunoglobulin connected with a short linker peptide. These scFv antibodies can bind selectively to a specific antigen or they can be engineered to be multifunctional by appending to the fusion protein- or nucleic acid-biding domains, such as for example, in the case of bispecific scFvs. Alternatively, mRNA encoding both VH and VL chains may be used. Additionally, a single-domain antibody (sdAb), consisting of a single monomeric variable domain, can be delivered to the cells and tissues of the eye as mRNAs. In additional embodiments, various other truncated antibodies and functional fragments thereof find use.

Also contemplated in the context of therapeutics for vision disorders is the targeted expression of antigenic peptides, or neoantigens, which can occur using ARMM-mediated delivery of an mRNA. The delivered mRNA is translated by the ribosome to produce a neoantigen protein chain which can be processed by the proteasome to produce a neoantigen. This neoantigen can associate with other membrane-bound proteins to display itself, thereby allowing it to be recognized by T-cell receptors on T-cells or other cells of the immune system.

In some aspects of this invention, arrestin domain-containing protein 1 (ARRDC1)-mediated microvesicles (ARMMs) containing a lipid bilayer and an ARRDC1 protein, a molecule (e.g., therapeutic agent(s)), and optionally a viral envelope protein are provided. In some aspects, the viral envelope protein is vesicular stomatitis virus G (VSV-G) or rabies virus glycoprotein (RVG).

In some aspects of the invention, microvesicle-producing cells containing a recombinant expression construct encoding an ARRDC1 protein or a variant thereof under the control of a heterologous promoter, and optionally, a viral envelope protein are provided. In other aspects, the viral envelope protein is vesicular stomatitis virus G (VSV-G) or rabies virus glycoprotein (RVG).

In some additional aspects of the invention, methods of delivering a molecule (e.g., one or more therapeutic agents) to a target cell type, tissue, or structure by contacting the target with a microvesicle, as described herein, are provided. In other aspects, the tissues and structures include the retina. In some aspects, the tissue or structure comprises an element of the retina, such as the Retinal Pigment Epithelium and the retinal nerve layer (e.g., retina ganglion cells, amacrine cells, bipolar cells, horizontal cells, photoreceptor cells, and the like).

In some aspects of the invention, methods of treating a disorder in a patient by administering to the patient a microvesicle or a microvesicle-producing cell as described herein are provided. In yet other aspects, the disorder impacts the function of the eye. In still other aspects, the disorder impacts the function of one or more of the retinae, sclera, choroid, iris, lens, cornea, vitreous humor, macula, optic nerve, and like structures, tissues, and cells, of the eye. In other aspects of the invention, the disorder is either a gain-of-function disorder, a loss-of-function disorder, or a repeat expansion. In still other aspects of the invention, the disorder results from one or more substitutions (e.g., missense or nonsense), insertions, deletions, deletion-insertions, duplications, inversions, frameshifts, or repeat expansions.

In some embodiments, time-dependent uptake of ARMMs in neural retina are obtained. In some embodiments, ocular related ARMMs compositions are effectively taken up by one or more of the subject's rods, cones, and retinal pigment epithelium. In certain other embodiments, the ocular related ARMMs compositions of the present invention do not cause deleterious cytotoxicity, leukocyte or macrophage infiltration, or cell death in the cells, tissues, and structures of the recipient's eye.

Other advantages, features, and uses of the invention will be apparent from the detailed description of certain exemplary, non-limiting embodiments, the drawings, the non-limiting working examples, and the claims.

### BRIEF DESCRIPTION OF THE DRAWINGS

FIG. 1 illustrates one representative embodiment of functional delivery of Cre recombinase enzyme to the retinal pigment epithelium and photoreceptors after subretinal injections in Ai14 mice. More particularly, FIG. 1A provides a schematic diagram of Cre activity report by a tdTomato-based expression cassette in the Rosa26 locus of Ai14 mice.

FIG. 1B illustrates the archetypal laminar organization of the vertebrate eye evident in a cross section. Distinct cell types occupy specific layers as depicted. In preferred methods, ARMMs compositions administered using subretinal injections are placed between one or more of the retinal pigmented epithelial cell layer, photoreceptor cells, ganglion cell layer, inner nuclear layer, and/or outer nuclear layer. FIG. 1C show representative confocal micrographs of cross sections from an approximately two month old uninjected Ai14 mouse (top row panels) and littermates subretinally injected with ARMMs payloaded with Cre as a fusion protein with ARRDC1 (A1-Cre) and harvested 10 days after injection (bottom row panels). The sections were stained with Hoechst to mark the nuclear layers and with a primary antibody against CRALBP to mark Mueller glia in the neural retina and the RPE cell layer. The arrows in the inset image, represent tdTomato-positive photoreceptor nuclei in the outer nuclear layer. Furthermore, regarding the inset image, herein is provided a detailed zoom of the outer nuclear layer and the retinal pigmented epithelial layer. Further, provided is a representative image selected from sections from five independent animals from two different litters that received two different lots of A1-cre test article on separate days. (Scale 50 microns.)

FIG. 2 illustrates results from conducting Example 2 wherein Göttingen minipigs were subretinally injected with ARMMs that were loaded with green fluorescent protein (ARRDC1-GFP-ARMMs) (SEQ ID NO: 50 and SEQ ID NO: 51).

FIG. 3 illustrates a cross section from a globe obtained from an animal in Example 2.

FIG. 4 illustrates results from conducting Example 3 wherein nonhuman primates, specifically, St. Kitt's African Green monkeys (Chlorocebus sabaeus) were subretinally injected with ARMMs that were loaded with mcherry (ARRDC1-mCherry) (SEQ ID NO: 52 and SEQ ID NO: 53).

FIG. 5 illustrates a cross section from a globe obtained from an animal in Example 3.

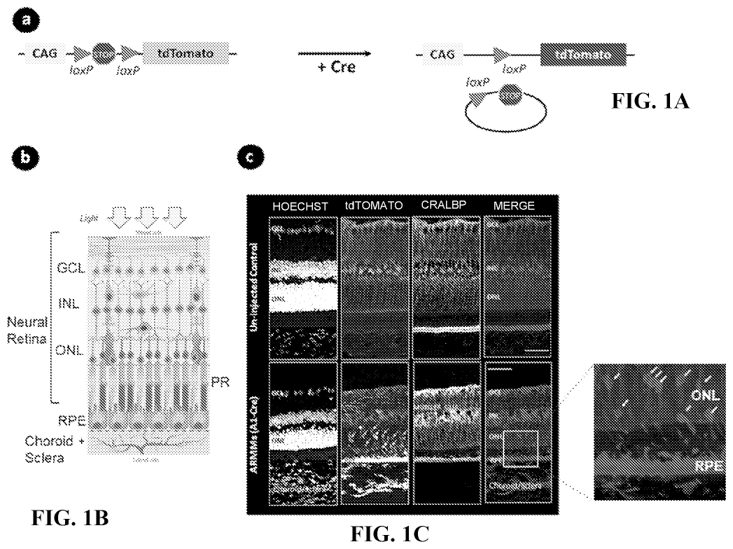

### DEFINITIONS

As used herein, the term “eye” refers to the organ of sight. The eye has several components including, but not limited to, the cornea, the iris, the pupil, the lens, the retina, the macula, the optic nerve, the choroid, and the vitreous.

As used herein, “ocular” can refer to the retina, the fundus, optic disc, macula, iris, pupil, lens, vessels, vitreous, or other eye-related anatomical components.

As used herein, the term “retina” refers to the classically-defined “neural retina” a nerve layer that lines the back of the eye, including cells (e.g., photoreceptors, amacrine cells, bipolar cells, horizontal cells, and ganglion cells) that are sensitive to light and that trigger nerve impulses that pass via the optic nerve to the brain, where a visual image is formed. The retina receives light and converts the light into neural signals. The support cells such as the Retinal Pigmented Epithelium (“RPE”), that directly participate in visual perception and provide support function to the neural retina are also included in the term “retina.”

The term “central retina,” as used herein, refers to the outer macula and/or inner macula and/or the fovea. The term “central retina cell types” as used herein refers to cell types of the central retina, such as, the cone photoreceptors, rod photoreceptors, retinal ganglion cells etc., and also the cells that directly support visual perception, for example, Retinal Pigment Epithelium that is anatomically juxtaposed to the photoreceptors of central retina.

As used herein, the term “Macula lutea” (“macula”) refers to a region of the central retina that contains a higher relative concentration of cone photoreceptor cells, relative to rod photoreceptors, compared to the peripheral retina. The term “outer macula” as used herein may also be referred to as the “peripheral macula.” The term “inner macula” as used herein may also be referred to as the “central macula.”

As used herein, the term “fovea” refers to a small region in the central retina of primates identifiable as a small depression of approximately equal to or less than 0.5 mm in diameter that exclusively contains cones, when compared to the peripheral retina and the macula.

As used herein, the term “subretinal space” refers to the location in the retina between the photoreceptor cells and the retinal pigment epithelium cells. The subretinal space may be a potential space, such as prior to any subretinal injection. In some embodiments, an amount (e.g., a therapeutic amount) of an ARRM-mediated composition of the present invention can be posited (e.g., via subretinal injection) into the subretinal space. In this case, the ARMMs-mediated composition is “in contact with the subretinal space.” Cells that are “in contact with the subretinal space” include the cells that border the subretinal space, such as RPE and photoreceptor cells.

As used herein, the term “bleb” refers to a fluid space within the subretinal space of an eve. A bleb of the invention may be created by a single injection of fluid into a single space, by multiple injections of one or more fluids into the same space, or by multiple injections into multiple spaces, which when repositioned create a total fluid space useful for achieving a therapeutic effect over the desired portion of the subretinal space.

As used herein, the term “optic nerve” refers to a connection between an eye and the brain. Each optic nerve is part of the second pair of cranial nerves. The optic nerve transmits impulses that are formed by the retina to the visual cortex of the brain, which interprets the impulses as images.

As used herein, the term “choroid” refers to the vascular layer of the eye, containing connective tissues, and lying between the retinal pigmented epithelium and the sclera.

The terms “ARRDC1-mediated microvesicle” or “ARMM,” as used herein, refers to a microvesicle comprising an ARRDC1 protein or variant thereof, and/or TSG101 protein, or variant thereof. ARMMs have been described in detail, for example, in PCT application number PCT/US2013/024839, filed Feb. 6, 2013 (published as WO 2013/119602 A1 on Aug. 15, 2013) by Lu et al., and entitled “Arrdc1-Mediated Microvesicles (ARMMs) and Uses Thereof,” as well as in U.S. Pat. Nos. 9,737,480; 9,816,080; 10,260,055, 10,945,954, 11,001,817, and PCT Publications WO2018/067546, WO2021/0662196, and WO2021/252924; the entire contents of which are hereby incorporated by reference in their entirety. In some embodiments, the ARMM is shed from a cell (e.g., producer cell), and comprises an agent (payload), for example, a nucleic acid, protein, or small molecule, present in the cytoplasm or associated with the membrane of the cell. Exemplary payloads include, but are not limited to a nucleic acid, protein, or small molecule, present in the cytoplasm or associated with the membrane of the cell. In some embodiments, the ARMM is shed from a cell (e.g., a transgenic cell), and comprises an agent, for example, a nucleic acid, protein, or small molecule, present in the cytoplasm or associated with the membrane of the cell. In some embodiments, the ARMM is shed from a transgenic cell comprising a recombinant expression construct that includes a transgene, and the ARMM comprises a gene product, for example, an RNA transcript and/or a protein (e.g., an ARRDC1-Tat fusion protein and a TAR-payload RNA) encoded by the expression construct. In some embodiments, the ARMM is produced synthetically, for example, by contacting a lipid bilayer with an ARRDC1 protein or a variant thereof, or a variant thereof, in a cell-free system in the presence of TSG101, or a variant thereof. In other embodiments, the ARMM is synthetically produced by contacting a lipid bilayer with HECT domain ligase, and VPS4a. In some embodiments, an ARMM lacks a late endosomal marker. Some of the ARMMs provided herein do not include, or are negative for, one or more exosomal biomarkers. Exosomal biomarkers are known to those of skill in the art and include, but are not limited to, CD63, Lamp-1, Lamp-2, CD9, HSPA8, GAPDH, CD81, SDCBP, PDCD6IP, ENO1, ANXA2, ACTB, YWHAZ, HSP90AA1, ANXA5, EEF1A1, YWHAE, PPIA, MSN, CFL1, ALDOA, PGK1, EEF2, ANXA1, PKM2, HLA-DRA, and YWHAB. Certain ARMMs provided herein may include an exosomal biomarker. Accordingly, some ARMMs may be negative for one or more other exosomal biomarkers, but positive for one or more different exosomal biomarkers. For example, such an ARMM may be negative for CD63 and Lamp-1 but may include PGK1 or GAPDH: or may be negative for CD63, Lamp-1, CD9, and CD81, but may be positive for HLA-DRA. In some embodiments, ARMMs include an exosomal biomarker, but at a lower level than the level found in exosomes. For example, some ARMMs include one or more exosomal biomarkers at a level of less than about 1%, less than about 5%, less than about 10%, less than about 20%, less than about 30%, less than about 40%, or less than about 50% of the level of that biomarker found in exosomes. To give a non-limiting example, in some embodiments, an ARMM may be negative for CD63 and Lamp-1, include CD9 at a level of less than about 5% of the level of CD9 typically found in exosomes, and be positive for ACTB. Exosomal biomarkers in addition to those listed above are known in the art, and the invention is not limited in this regard.

The term “cargo protein,” as used herein, refers to a protein that may be incorporated in an ARMM, for example, into the liquid phase of the ARMM or into the lipid bilayer of an ARMM. The term “cargo protein to be delivered” refers to any protein that can be delivered via its association with or inclusion in an ARMM to a subject, organ, tissue, or cell. In some embodiments, the cargo protein is to be delivered to a target cell in vitro, in vivo, or ex vivo. In some embodiments, the cargo protein to be delivered is a biologically active agent, i.e., it has activity in a cell, organ, tissue, and/or subject. For instance, a protein that, when administered to a subject, has a biological effect on that subject, is considered biologically active. In certain embodiments, the cargo protein is a nuclease or variant thereof (e.g., a Cas9 protein or variant thereof). In certain embodiments, the nuclease may be a Cas9 nuclease, a TALE nuclease, a zinc finger nuclease, or any variant thereof. Nucleases, including Cas9 proteins and their variants, are described in more detail elsewhere herein. In some embodiments, the Cas9 protein or variant thereof is associated with nucleic acid. For example, the cargo protein may be a Cas9 protein associated with a gRNA. In some embodiments, a cargo protein to be delivered is a therapeutic agent.

As used herein, the term “therapeutic agent” refers to any agent that, when administered to a subject has a beneficial effect. In some embodiments, the therapeutic agent comprises a small molecule, a protein (or peptide), one or more nucleic acids, or an agent associated with a small molecule. In some embodiments, the payload to be delivered is a diagnostic agent. In some embodiments, the agent to be delivered is a prophylactic agent. In some embodiments, the agent to be delivered is useful as an imaging agent. In some of these embodiments, the diagnostic or imaging agent is, and in others, it is not, biologically active. In some embodiments, the therapeutic agent comprises an agent that reduces (knocks down) the expression of one or more genes in an organism (e.g., a subject). In other embodiments, the therapeutic agent comprises an agent that inactivates or removes (knocks out) one or more specific genes in an organism (e.g., a subject). In some embodiments, the therapeutic agent to be delivered to a cell is a transcription factor, a tumor suppressor, a developmental regulator, a growth factor, a metastasis suppressor, a pro-apoptotic protein, a nuclease, or a recombinase.

As used herein, the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

As used herein, the term “transcription factor” refers to a DNA-binding protein that regulates the transcription of DNA into RNA, for example, by activation or repression of transcription. Some transcription factors affect the regulation of transcription alone, while others act in concert with other proteins. Some transcription factors can both activate and repress transcription under certain conditions. In general, transcription factors bind a specific target sequence or sequences highly similar to a specific consensus sequence in a regulatory region of a target gene. Transcription factors may regulate the transcription of a target gene alone or in a complex with other molecules. Examples of transcription factors include, but are not limited to, Sp1, NF1, CCAAT, GATA, HNF, PIT-1, MyoD, Myf5, Hox, Winged Helix, SREBP, p53, CREB, AP-1, Mef2, STAT, R-SMAD, NF-Kβ, Notch, TUBBY, and NFAT.

The term “binding RNA,” as used herein, refers to a ribonucleic acid (RNA) that binds to an RNA binding protein, for example, any of the RNA binding proteins known in the art and/or described herein. In some embodiments, a binding RNA is an RNA that specifically binds to an RNA binding protein. A binding RNA that “specifically binds” to an RNA binding protein, binds to the RNA binding protein with greater affinity, avidity, more readily, and/or with greater duration than it binds to another protein, such as a protein that does not bind the RNA or a protein that weakly binds to the binding RNA. In some embodiments, the binding RNA is a naturally occurring RNA, or non-naturally occurring variant thereof, that binds to a specific RNA binding protein. For example, the binding RNA may be a TAR element, a Rev response element, an MS2 RNA, or any variant thereof that specifically binds an RNA binding protein. In some embodiments, the binding RNA may be a trans-activating response element (TAR element), or variant thereof, which is an RNA stem-loop structure that is found at the 5′-ends of nascent HIV-1 transcripts and specifically binds to the trans-activator of transcription (Tat) protein. In some embodiments, the binding RNA is a Rev response element (RRE), or variant thereof, that specifically binds to the accessory protein Rev (e.g., Rev from HIV-1). In some embodiments, the binding RNA is an MS2 RNA that specifically binds to a MS2 phage coat protein. The binding RNAs of the present disclosure may be designed to specifically bind a protein (e.g., an RNA binding protein fused to ARRDC1) to facilitate loading of the binding RNA (e.g., a binding RNA fused to a payload RNA) into an ARMM.

The term “aptamer,” as used herein, refers to nucleic acids (e.g., RNA, DNA) that bind to a specific target molecule, e.g., an RNA binding protein. In some embodiments, nucleic acid (e.g., DNA or RNA) aptamers are engineered through repeated rounds of in vitro selection or alternatively, SELEX (systematic evolution of ligands by exponential enrichment) methodology, to bind to various molecular targets, for example, proteins, small molecules, macromolecules, metabolites, carbohydrates, metals, nucleic acids, cells, tissues, and organisms. Methods for engineering aptamers to bind to various molecular targets, such as proteins, are known in the art and include those described in U.S. Pat. Nos. 6,376,19; and 9,061,043; Shui, B., et al., “RNA aptamers that functionally interact with green fluorescent protein and its derivatives,” Nucleic Acids Res., Mar; 40(5): e39 (2012); Trujillo, U. H., et al., “DNA and RNA aptamers: from tools for basic research towards therapeutic applications.” Comb. Chem. High Throughput Screen, 9(8):619-32 (2006); Srisawat, C., et al., “Streptavidin aptamers: Affinity tags for the study of RNAs and ribonucleoproteins,” RNA, 7:632-641 (2001); and Tuerk and Gold, “Systematic evolution of ligands by exponential enrichment: RNA ligands to bacteriophage T4 DNA polymerase,” Science, (1990); the entire contents of each of which are hereby incorporated by reference in their entirety.

The term “RNA binding protein,” as used herein, refers to a polypeptide molecule that binds to a binding RNA, for example, any of the binding RNAs known in the art and/or described herein. In some embodiments, an RNA binding protein is a protein that specifically binds to a binding RNA. An RNA binding protein that “specifically binds” to a binding RNA, binds to the binding RNA with greater affinity, avidity, more readily, and/or with greater duration than it binds to another RNA, such as a control RNA (e.g., an RNA having a random nucleic acid sequence) or an RNA that weakly binds to the RNA binding protein. In some embodiments, the RNA binding protein is a naturally occurring protein or a non-naturally occurring variant thereof, that binds to a specific RNA. For example, in some embodiments, the RNA binding protein may be a trans-activator of transcription (Tat) protein that specifically binds a trans-activating response element (TAR element). In some embodiments, the Tat protein is from a bovine. In some embodiments, the RNA binding protein is a regulator of virion expression (Rev) protein (e.g., Rev from HIV-1) or variant thereof, that specifically binds to a Rev response element (RRE). In some embodiments, the RNA binding protein is a coat protein of an MS2 bacteriophage that specifically binds to an MS2 RNA. The RNA binding proteins useful in the present disclosure (e.g., a binding protein fused to ARRDC1) may be designed to specifically bind a binding RNA (e.g., a binding RNA fused to a payload RNA) to facilitate loading of the binding RNA into an ARMM.

The term “payload,” “payload protein,” “payload nucleic acid,” “payload DNA,” “payload RNA,” or “payload small molecule,” as used herein, refers to a protein, nucleic acid, including DNA or RNA, or a small molecule, respectively, that may be incorporated into an ARMM, for example, into the liquid phase of the ARMM or into the lipid bilayer of an ARMM. Types of payload protein, payload nucleic acid, payload DNA, payload RNA, and payload small molecule are known in the art and include those described in U.S. Pat. Nos. 9,737,480; 9,816,080; 10,260,055; and PCT Publication WO2018/067546; the entire contents of each of which are hereby incorporated by reference in their entirety.

The payload can be delivered via its association with or inclusion in an ARMM to a subject, organ, tissue, or cell. In some embodiments, the payload is to be delivered to a targeted cell in vitro, in vivo, or ex vivo. In some embodiments, the payload to be delivered is a biologically active agent, i.e., it has activity in a cell, organ, tissue, and/or subject. For instance, a protein, nucleic acid (e.g., DNA or RNA), or small molecule that, when administered to a subject, has a biological effect on that subject or is biologically active. In some embodiments, a payload to be delivered is a therapeutic agent.

The term “viral envelope proteins” refers to proteins that normally function to aid viral attachment and entry into cells. In some embodiments, viral envelope proteins can be incorporated into ARMMS to allow for the targeting of cells of the eve. Non-limiting examples of viral envelope proteins include vesicular stomatitis virus G protein (VSV-G) or rabies virus glycoprotein (RVG). VSV-G mediates viral attachment to LDL receptors (LDLR) or LDLR family members, and RVG is known to use the nicotinic acetylcholine receptor and the low affinity nerve growth factor receptor for viral entry.

The term “linker,” as used herein, refers to a chemical moiety linking two molecules or moieties, e.g., an ARRDC1 protein and a Tat protein, a WW domain, and a Tat protein, or an ARRDC1 protein and a Cas9 nuclease. Typically, the linker is positioned between, or flanked by, two groups, molecules, or other moieties and connected to each one via a covalent bond, thus connecting the two. In some embodiments, the linker comprises an amino acid or a plurality of amino acids (e.g., a peptide or protein). In some embodiments, the linker comprises a nucleotide (e.g., DNA or RNA) or a plurality of nucleotides (e.g., a nucleic acid). In some embodiments, the linker is an organic molecule, functional group, polymer, or other chemical moiety/moieties. In some embodiments, the linker is a cleavable linker, e.g., the linker comprises a bond that can be cleaved upon exposure to, for example, UV light or a hydrolytic enzyme, such as a protease or esterase. In some embodiments, the linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids). In other embodiments, the linker is a chemical bond (e.g., a covalent bond, amide bond, disulfide bond, ester bond, carbon-carbon bond, carbon-heteroatom bond, and the like).

As used herein, the term “animal” refers to any member of the animal kingdom. In some embodiments, the term “animal” refers to a human of either sex at any stage of development. In some embodiments, the term “animal” refers to a non-human animal at any stage of development. In certain embodiments, the non-human animal is a mammal (e.g., a rodent, a mouse, a rat, a rabbit, a monkey, a dog, a cat, a sheep, cattle, a primate, or a pig). Animals include, but are not limited to, mammals, birds, reptiles, amphibians, fish, and worms. In some embodiments, the animal is a transgenic animal, a genetically engineered animal, or a clone. In some embodiments, the animal is a transgenic non-human animal, a genetically engineered non-human animal, or a non-human clone.

As used herein, the terms “associated with,” “conjugated,” “linked,” “attached,” and the like, when used with respect to two or more entities, for example, with chemical moieties, molecules, and/or ARMMs, means that the entities are physically associated or connected with one another, either directly or via one or more additional moieties that serve as a linker, to form a structure that is sufficiently stable so that the entities remain physically associated under the conditions in which the structure is used, e.g., under physiological conditions. An ARMM microvesicle is typically associated with an agent, for example, a nucleic acid, protein, or small molecule, by a mechanism that involves a covalent (e.g., via an amide bond) or non-covalent association (e.g., between ARRDC1 and a WW domain, or between a Tat protein and a TAR element). In certain embodiments, the agent (e.g., a therapeutic agent, a payload protein, payload nucleic acid, or payload small molecule) is covalently bound to a molecule that associates non-covalently with a part of the ARMM that is fused to an ARRCD1 protein, a TSG01 protein or variant thereof, or a lipid bilayer-associated protein by a covalent bond (e.g., an amide bond), or variant thereof. In some embodiments, the association is via a linker, for example, a cleavable linker. In some embodiments, an entity (e.g., a payload protein, payload nucleic acid, or payload small molecule) is associated with an ARMM by inclusion in the ARMM, for example, by encapsulation of the molecule within the ARMM. For example, in some embodiments, a molecule (e.g., a therapeutic agent, a payload protein, payload nucleic acid, or payload small molecule) present in the cytoplasm of an ARMM-producing cell is associated with an ARMM by encapsulation of the cytoplasm with the agent in the ARMM upon ARMM budding. Similarly, a membrane protein or other molecule associated with the cell membrane of an ARMM producing cell may be associated with an ARMM produced by the cell by inclusion into the ARMM's membrane upon budding.

As used herein, the phrase “biologically active” refers to a characteristic of any substance that has activity in a cell, organ, tissue, and/or subject. For instance, a substance that when administered to an organism produces or elicits a biological effect on that organism is biologically active. As one example, a payload RNA may be considered biologically active if it increases or decreases the expression of a gene product when administered to a subject or cell. As another example, a nuclease payload protein may be considered biologically active if it increases or decreases the expression of a gene product when administered to a subject.

As used herein, the term “conserved” refers to nucleotides or amino acid residues of a polynucleotide sequence or amino acid sequence, respectively, that are those that occur unaltered in the same position of two or more related sequences being compared. Nucleotides or amino acids that are relatively conserved are those that are conserved amongst more related sequences than nucleotides or amino acids appearing elsewhere in the sequences. In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95% identical, about 98% identical, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another.

The term “engineered,” as used herein, refers to a protein, nucleic acid, complex, substance, or entity that has been designed, produced, prepared, synthesized, and/or manufactured by a human. Accordingly, an engineered product is a product that does not occur in nature. In some embodiments, an engineered protein or nucleic acid is a protein or nucleic acid that has been designed to meet requirements or to have desired features. For example, a payload RNA may be engineered to associate with the ARRDC1 by fusing one or more WW domains to a Tat protein and fusing the payload RNA to a TAR element to facilitate loading of the payload RNA into an ARMM. As another example, a payload RNA may be engineered to associate with the ARRDC1 by fusing a Tat protein to the ARRDC1 and by fusing the payload RNA to a TAR element to facilitate loading of the payload RNA into an ARMM. As another example, a payload protein may be engineered to associate with the ARRDC1 by fusing one or more WW domains to the payload protein to facilitate the loading of the payload protein into an ARMM.

As used herein, the term “expression” of a nucleic acid sequence refers to one or more of the following events: (1) production of an RNA transcript from a DNA sequence (e.g., by transcription); (2) processing of an RNA transcript (e.g., by splicing, editing, 5′ cap formation, and/or 3′ end processing); (3) translation of an RNA transcript into a poly peptide or protein; and (4) post-translational modification of a polypeptide or protein.

The term “operably linked.” as used herein, refers to an arrangement of sequences or regions wherein the components are configured to perform their usual or intended function. Thus, a regulatory or control sequence operably linked to a coding sequence is capable of affecting the expression of the coding sequence. The regulatory or control sequences need not be contiguous with the coding sequence, so long as they function to direct the proper expression or polypeptide production. Thus, for example, intervening untranslated but transcribed sequences can be present between a promoter sequence and the coding sequence and the promoter sequence can still be considered operably linked to the coding sequence. A promoter sequence, as described herein, is a DNA regulatory region a short distance from the 5′ end of a gene that acts as the binding site for RNA polymerase. The promoter sequence may bind RNA polymerase in a cell and/or initiate transcription of a downstream (3′ direction) coding sequence. The promoter sequence may be a promoter capable of initiating transcription in prokaryotes or eukaryotes. Some non-limiting examples of eukaryotic promoters include the cytomegalovirus (CMV) promoter, the chicken f-actin (CBA) promoter, and a hybrid form of the CBA promoter (CBh).

As used herein, a “fusion protein” includes a first protein moiety, e.g., an ARRCD1 protein or variant thereof, or a TSG101 protein or variant thereof, associated with a second protein moiety, for example, a protein to be delivered to a target cell through a peptide linkage. In certain embodiments, the fusion protein is encoded by a single fusion gene.

As used herein, the term “gene” has its meaning as understood in the art. It will be appreciated by those of ordinary skill in the art that the term “gene” may include gene regulatory sequences (e.g., promoters, enhancers, etc.) and/or intron sequences. It will further be appreciated that the definition of a gene includes references to nucleic acids that do not encode proteins but rather encode functional RNA molecules, such as gRNAs, RNAi agents, ribozymes, tRNAs, etc. It should be noted that, as used in the present application, the term “gene” generally refers to a portion of a nucleic acid that encodes a protein: the term may optionally encompass regulatory sequences, as will be clear from context to those of ordinary skill in the art. This definition is not intended to exclude the application of the term “gene” to non-protein-coding expression units but rather to clarify that, in most cases, the term as used herein refers to a protein-coding nucleic acid.

As used herein, the term “gene product” or “expression product” generally refers to an RNA transcribed from the gene (pre-and/or post-processing) or a polypeptide (pre- and/or post-modification) encoded by an RNA transcribed from the gene.

As used herein, the term “green fluorescent protein” (“GFP”) refers to a protein originally isolated from the jellyfish Aequorea victoria that fluoresces green when exposed to blue light or a derivative of such a protein (e.g., an enhanced or wavelength-shifted version of the protein). The amino acid sequence of wild-type GFP is as follows:

(SEQ ID NO: 3)
MSKGEELFTGVVPILVELDGDVNGHKESVSGEGEGDATYGKLTLKFICTT
GKLPVPWPTLVTTFSYGVQCFSRYPDHMKQHDFFKSAMPEGYVQERTIFFK
DDGNYKTRAEVKFEGDTLVNRIELKGIDFKEDGNILGHKLEYNYNSHNVYI
MADKQKNGIKVNFKIRHNIEDGSVQLADHYQQNTPIGDGPVLLPDNHYLST
QSALSKDPNEKRDHMVLLEFVTAAGITHGMDELYK

Proteins that are at least 70%, at least 75%, at least 80%, at least 85%, at least 90%, at least 95%, at least 98%, or at least 99% homologous to SEQ ID NO: 3 are also considered to be green fluorescent proteins.

As used herein, the term “homology” refers to the overall relatedness between nucleic acids (e.g., DNA molecules and/or RNA molecules) or polypeptides. In some embodiments, nucleic acids or proteins are considered to be “homologous” to one another if their sequences are at least 50%, at least 55%, at least 60%, at least 65%, at least 70%, at least 75%, at least 80%, at least 85%, at least 90%, at least 95%, or at least 99% identical. In some embodiments, nucleic acids or proteins are considered “homologous” to one another if their sequences are at least 50%, at least 55%, at least 60%, at least 65%, at least 70%, at least 75%, at least 80%, at least 85%, at least 90%, at least 95%, or at least 99% identical. The term “homologous” necessarily refers to a comparison between at least two sequences (nucleotide sequences or amino acid sequences). In accordance with the invention, two nucleotide sequences are considered homologous if the polypeptides they encode are at least about 50% identical, at least about 60% identical, at least about 70% identical, at least about 80% identical, or at least about 90% identical for at least one stretch of at least about 20 amino acids. In some embodiments, homologous nucleotide sequences are characterized by the ability to encode a stretch of at least 4-5 uniquely specified amino acids. Both the identity and the approximate spacing of these amino acids relative to one another must be considered for sequences to be considered homologous. For nucleotide sequences less than 60 nucleotides in length, homology is determined by the ability to encode a stretch of at least 4-5 uniquely specified amino acids. In accordance with the invention, two protein sequences are considered homologous if the proteins are at least about 50% identical, at least about 60% identical, at least about 70% identical, at least about 80% identical, or at least about 90% identical for at least one stretch of at least about 20 amino acids.

As used herein, the term “identity” refers to the overall relatedness between nucleic acids or proteins (e.g., DNA molecules, RNA molecules, and/or polypeptides). Calculation of the percent identity of two nucleic acid sequences, for example, can be performed by aligning the two sequences for optimal comparison purposes (e.g., gaps can be introduced in one or both of a first and second nucleic acid sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smth, D. W., ed., Academic Press, New York, 1993; Sequence Analysis in Molecular Biology, von Heinje, G., Academic Press, 1987: Computer Analysis of Sequence Data, Part I, Griffin, A. M., and Griffin, H. G., eds., Humana Press, New Jersey. 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo. H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research. 12(1), 387 (1984)), BLASTP, BLASTN, and FASTA Atschul, S. F., et al., J. Molec. Biol., 215, 403 (1990)).

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe).

As used herein, the term “ex vivo” refers to events outside of the living body and thusly is understood to refer to medical procedures in which an organ, cells, or tissue is taken from a living body for a treatment or procedure, and then returned to the same, or another, living body. In certain embodiments, ex vivo therapy comprises inducing one or more genetic modifications in a patient's cells outside of their body to produce therapeutic effects therein and the subsequent transfer (e.g., transplantation) of the cells back into the patient.

As used herein, the term “isolated” refers to a substance or entity that has been: (1) separated from at least some of the components with which it was associated when initially produced (whether in nature or in an experimental setting); and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, or more of the other components with which they were initially associated. In some embodiments, isolated substances are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

As used herein, the term “nucleic acid,” in its broadest sense, refers to a compound and/or substance that is or can be incorporated into an oligonucleotide chain via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising individual nucleotides. As used herein, the terms “oligonucleotide” and “polynucleotide” can be used interchangeably to refer to a polymer of nucleotides (e.g., a string of at least two nucleotides). In some embodiments, “nucleic acid” encompasses RNA as well as single and/or double-stranded DNA and/or complementary DNA (cDNA). Furthermore, the terms “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. The term “nucleic acid segment” is used herein to refer to a nucleic acid sequence that is a portion of a longer nucleic acid sequence. In many embodiments, a nucleic acid segment comprises at least 3, at least 4, at least 5, at least 6, at least 7, at least 8, at least 9, at least 10, or more residues. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases: biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention is specifically directed to “unmodified nucleic acids,” meaning nucleic acids (e.g., polynucleotides and residues, including nucleotides and/or nucleosides) that have not been chemically modified to facilitate or achieve delivery.

As used herein, the term “protein” refers to a string of at least two amino acids linked to one another by one or more peptide bonds. Proteins may include moieties other than amino acids (e.g., may be glycoproteins) and/or may be otherwise processed or modified. Those of ordinary skill in the art will appreciate that a “protein” can be a complete protein chain as produced by a cell (with or without a signal sequence) or can be a functional portion thereof. Those of ordinary skill will further appreciate that a protein can sometimes include more than one protein chain, for example linked by one or more disulfide bonds or associated by other means. Proteins may contain L-amino acids. D-amino acids, or both and may contain any of a variety of amino acid modifications or analogs known in the art. Useful modifications include, e.g., addition of a chemical entity such as a carbohydrate group, a phosphate group, a farnesyl group, an isofarnesyl group, a fatty acid group, an amide group, a terminal acetyl group, a linker for conjugation, functionalization, or other modification (e.g. alpha amidation), etc. In certain embodiments, the modifications of the protein lead to a more stable protein (e.g., greater half-life in vivo). These modifications may include cyclization of the protein, the incorporation of D-amino acids, etc. None of the modifications should substantially interfere with the desired biological activity of the protein. In certain embodiments, the modifications of the protein lead to a more biologically active protein. In some embodiments, proteins may comprise natural amino acids, non-natural amino acids, synthetic amino acids, amino acid analogs, and combinations thereof.

As used herein, the terms “subject,” or “patient” refer to any organism to which a composition in accordance with the invention may be administered, e.g., for experimental, diagnostic, prophylactic, and/or therapeutic purposes. Typical subjects include animals (e.g., mammals, such as mice, rats, rabbits, production and farm animals, pets, non-human primates, and humans). In some embodiments, the subject is a patient having or suspected of having a disease or disorder. In other embodiments, the subject is a healthy volunteer.

As used herein, the terms “disease,” and “disorder” refer to any condition, pathological condition, or disorder that damages or interferes with the normal function of a cell, tissue, or organ.

As used herein, the terms “retinal disease,” and “retinal disorder,” and equivalents, refer to any disease, disorder, or symptom related to the retina, examples include, but are not limited to, retinal degenerative diseases (e.g., retinitis pigmentosa, age-related macular degeneration, etc.), retinopathies (e.g., diabetic retinopathy, proliferative retinopathy, simple retinopathy, etc.), and the like. Herein, the compositions and methods of the present invention are capable of preventing, treating, or suppressing the progression of diseases or disorders including, but not limited to, retinal degenerative diseases, age-related macular degeneration, myopic maculopathy, macular dystrophy, diabetic retinopathy, uveitis, and the like. Examples of the disorder or symptom include disorders in visual acuity, contrast sensitivity, light-dark adaptation, color vision, etc., and symptoms associated therewith.

As used herein, a “retinal degenerative disease” refers to any disease caused by degeneration of the retina, and examples include, but are not limited to, retinitis pigmentosa. MERTK retinitis pigmentosa, age-related macular degeneration, and the like.

The term “improvement in vision,” as used herein, refers to improving or recovering vision-related capabilities (e.g., visual acuity, color perception, contrast sensitivity, light-dark adaptation, etc.). For example, regarding visual acuity, acuity can be measured by a Snellen chart or an E chart in addition to a visual acuity test using a Landoldt ring and can be expressed by decimal visual acuity or fractional visual acuity. These can also be displayed with log MAR visual acuity. In mice, visual acuity can be measured using visual stimuli that manipulate the spatial frequencies of light and dark stripes. The visual acuity can also be determined by measuring the visual evoked potential.

As used herein, the terms “treating,” or “treatment” refer to partially or completely preventing, altering, and/or reducing the incidence of one or more symptoms or features of a particular disease or deleterious condition. In one sense of the invention, treatments can be performed either for prophylaxis or amelioration of a pathological condition in a subject. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. Treatment may be administered to a subject who does not exhibit signs or symptoms of a disease, disorder, condition, or to a subject who exhibits only early signs or symptoms of a disease, or condition for the purpose of decreasing the risk of developing or progressing to more severe effects associated with the disease, disorder, or condition. A treatment may prevent the onset of the disorder or a symptom of the disorder in a subject. A treatment can prevent physical deterioration (e.g., loss of vision, loss of visual acuity, low vision, blindness) caused by a disorder (e.g., inherited retinal dystrophies, Stargardt Macular Dystrophy. Choroideremia, Usher Syndrome 1b or 1c, MERTK Retinitis pigmentosa, macular edema, and the like) by preventing or reversing its progression.

As used herein, the term “therapeutically effective amount” means an amount of an agent or payload to be delivered (e.g., nucleic acid, protein, drug, therapeutic agent, diagnostic agent, prophylactic agent, ARMM, or ARMM comprising a payload protein or payload RNA) that is sufficient, when administered to a subject suffering from or susceptible to a disease, disorder, and/or condition, to treat, improve symptoms thereof, diagnose, prevent, and/or delay the onset of the disease, disorder, and/or condition. A therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference in its entirety.

As used herein, the term “targeting ligand” refers to a ligand, or a function portion thereof, that binds to “a targeted receptor” that distinguishes the cell being targeted from other cells. The ligands may be capable of binding due to expression or preferential expression of a receptor for the ligand, accessible for ligand binding, on the target cells. Examples of such ligands include GE11 peptide, anti-EGFR nanobody, cRGD (cyclo (RGDfC), KE108 peptide, octreotide, prostate-specific membrane antigen (PSMA) aptamer, TRC105, chimeric monoclonal antibodies, tumor-specific monoclonal or polyclonal antibodies (e.g., Rituximab, Trastuzumab, Bevacizumab, Alemtuzumab, Panitumumab, etc., and bioequivalents and portions thereof), arginylglycylaspartic acid (“RGD”), DARPins, RNA aptamers, DNA aptamers, inteins, exteins, viral derived and nonviral derived cell-cell fusion protein (“fusogen(s)”) (e.g., VSV-G, syncytin-1, -2, HAP2, SNAREs (e.g., VAMP1, 2, 3, 4, 7, 8)), membrane proteins, such as tetraspanins (“TM4SF proteins”) (e.g., TSPAN1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, and 33, and the like), peptide ligands identified from library screens, tumor-specific peptides, tumor-specific aptamers, Fab or scFv (i.e., a single chain variable region) fragments of antibodies such as, for example, an Fab fragment of an antibody directed to EphA2 or other proteins specifically expressed or uniquely accessible on metastatic cancer cells, growth factors, such as EGF, FGF, insulin, and insulin-like growth factors, and homologous polypeptides, somatostatin and its analogs, transferrin, lipoprotein complexes, Arg-Gly-Asp containing peptides, microtubule-associated sequence (MTAS), various galectins, δ-opioid receptor ligands, cholecystokinin A receptor ligands, ligands specific for angiotensin AT1 or AT2 receptors, peroxisome proliferator-activated receptor γ ligands, and other molecules that bind specifically to a receptor preferentially expressed on the surface of targeted cells or on an infectious organism, and fragments of any of these molecules.

The term “a targeted receptor,” as used herein, refers to a receptor expressed by a cell that can bind a cell targeting ligand. The receptor may be expressed on the surface of the cell. The receptor may be a transmembrane receptor. Examples of such targeted receptors include, but are not limited to, EGFR, αᵥβ₃ integrin, somatostatin receptor, folate receptor, prostate-specific membrane antigen, CD105, mannose receptor, estrogen receptor, GM1 ganglioside, and the like.

In some embodiments, cell penetrating peptides may also be attached to one or more PEG terminal groups in place of or in addition to the targeting ligand. As used herein, the terms “cell penetrating peptide” (“CPP”), “protein transduction domain” (“PTD”), or “membrane translocating sequence,” refer to short peptides (e.g., from 4 to about 40 amino acids) that have the ability to translocate across a cellular membrane to gain access to the interior of a cell and to carry into the cells a variety of covalently and noncovalently conjugated cargoes, including proteins, and oligonucleotides. In preferred embodiments, CPPs comprise: 1) a high relative abundance of positively charged amino acids (e.g., lysine or arginine); 2) an amino acid sequence that comprises an alternating pattern of polar, charged amino acids and non-polar, hydrophobic amino acids; or 3) an amino acid sequence that comprises hydrophobic peptides (e.g., mostly apolar residues with low net charge or hydrophobic amino acid groups). (See, e.g., US Pat. Pub. No.: 20220177494; Oliveira, E. C, et al., “Predicting cell-penetrating peptides using machine learning algorithms and navigating in their chemical space,” Scientific Reports., 11(1):7628 (2021); Derakhshankhah. H., and Jafari, S., “Cell penetrating peptides: A concise review with emphasis on biomedical applications,” Biomedicine & Pharmacotherapy., 108:1090-1096 (2018); Milletti, F., “Cell-penetrating peptides: classes, origin, and current landscape,” Drug Discovery Today, 17(15-16):850-860 (2012); Stalmans, S., et al., “Chemical-functional diversity in cell-penetrating peptides.” PLOS ONE, 8(8):e71752 (2013); Wagstaff, K. M., and Jans, D. A., “Protein transduction: cell penetrating peptides and their therapeutic applications,” Current Medicinal Chemistry, 13(12):1371-1387 (2006), the entire contents of each of which are hereby incorporated by reference in their entirety). Examples of CPP peptides include, but are not limited to: TAT cell penetrating peptide; MAP; Penetratin or Antenapedia PTD; Penetratin-Arg; antitrypsin (358-374); Temporin L; Maurocalcine; pVEC (Cadherin-5); Calcitonin; Neurturin; Penetratin; TAT-HA2 Fusion Peptide; TAT (47-57); SynB1; SynB3; PTD-4; PTD-5; FHV Coat-(35-49); BMV Gag-(7-25); HTLV-II Rex-(4-16); HIV-1 Tat (48-60) or D-Tat: R9-Tat; Transportan; SBP or Human P1; FBP; MPG(δNLS); Pep-1 or Pep-1-Cysteamine; Pep-2; Periodic sequences, Polyarginines (RxN (4<N<17) chimera); Polylysines (KxN (4<N<17) chimera); (RAca)6R; (RAbu)6R; (RG)6R; (RM)6R; (RT)6R; (RS)6R; R10; (RA)6R; and R7.

As used herein, a “vector” means any nucleic acid or nucleic acid-bearing particle, cell, or organism capable of being used to transfer a nucleic acid into a host cell. The term “vector” includes both viral and nonviral products and means for introducing the nucleic acid into a cell. A “vector” can be used in vitro, ex vivo, or in vivo. Vectors capable of directing the expression of operatively linked genes are referred to herein as “expression vectors.” Non-viral vectors include plasmids, cosmids, artificial chromosomes (e.g., bacterial artificial chromosomes or yeast artificial chromosomes), liposomes, electrically charged lipids (cytofectins). DNA-protein complexes, and biopolymers, for example. Viral vectors include, but are not limited to, retroviruses, lentiviruses, adeno-associated virus, pox viruses, baculovirus, reoviruses, vaccinia viruses, herpes simplex viruses, Epstein-Barr viruses, and adenovirus vectors, for example. Vectors can also comprise the entire genome sequence or recombinant genome sequence of a virus. A vector can also comprise a portion of the genome that comprises the functional sequences to produce a virus capable of infecting, entering, or being introduced to a cell to deliver nucleic acid therein.

The term “WW domain” as used herein, refers to a protein domain having two basic residues at the C-terminus that mediates protein-protein interactions with short proline-rich or proline-containing motifs. It should be appreciated that the two basic residues (e.g., any two of: H, R, and K) of the WW domain are not required to be at the absolute C-terminal end of the WW protein domain. Rather, the two basic residues may be at a C-terminal portion of the WW protein domain (e.g., the C-terminal half of the WW protein domain). In some embodiments, the WW domain contains at least 1, 2, 3, 4, 5, 6, 7, 8, 9, or 10 tryptophan (W) residues. In some embodiments, the WW domain contains at least two W residues. In some embodiments, the at least two W residues are spaced apart by from 15-25 amino acids. In some embodiments, the at least two W residues are spaced apart by from 19-23 amino acids. In some embodiments, the at least two W residues are spaced apart by from 20-22 amino acids. The WW domain possessing the two basic C-terminal amino acid residues may have the ability to associate with short proline-rich or proline-containing motifs (e.g., a PPXY (SEQ ID NO: 2) motif). WW domains bind a variety of distinct peptide ligands including motifs with core proline-rich sequences, such as PPXY (SEQ ID NO: 2), which is found in ARRDC1. A WW domain may be a 30-40 amino acid protein interaction domain with two signature tryptophan residues spaced by 20-22 amino acids. The three-dimensional structure of WW domains shows that they generally fold into a three-stranded, antiparallel R sheet with two ligand-binding grooves.

WW domains are found in many eukaryotes and are present in approximately 50 human proteins (Bork, P. & Sudol, M., “The WW domain: a signaling site in dystrophin?” Trends Biochem Sci., 19, 531-533 (1994)). WW domains may be present together with several other interaction domains, including membrane targeting domains, such as C2 in the NEDD4 family proteins, the phosphotyrosine-binding (PTB) domain in FE65 protein, FF domains in CA150 and FBPI1, and pleckstrin homology (PH) domains in PLEKHA5. WW domains are also linked to a variety of catalytic domains, including HECT E3 protein-ubiquitin ligase domains in NEDD4 family proteins, rotomerase or peptidyl prolyisomerase domains in Pinl, and Rho GAP domains in ArhGAP9 and ArhGAP12.

The WW domain may be a WW domain that naturally possesses two basic amino acids at the C-terminus. In some embodiments, a WW domain or WW domain variant may be from the human ubiquitin ligase WWP1, WWP2, Nedd4-1, Nedd4-2. Smurf1, Smurf2. ITCH, NEDL1, or NEDL2. Exemplary amino acid sequences of WW domain containing proteins (WW domains underlined) are listed below. It should be appreciated that any of the WW domains or WW domain variants of the exemplary proteins may be used in the invention, described herein, and are not meant to be limiting.

Human WWP1 amino acid sequence (uniprot.org/uniprot/Q9HOMO). The four underlined WW domains correspond to amino acids 349-382 (WW1), 381-414 (WW2), 456-489 (WW3), and 496-529 (WW4).

(SEQ ID NO: 4)
MATASPRSDT SNNHSGRLQL QVTVSSAKLK RKKNWFGTAI YTEVVVDGEI 50
TKTAKSSSSS NPKWDEQLTV NVTPQTTLEF QVWSHRTLKA DALLGKATID 100
LKQALLIHNR KLERVKEQLK LSLENKNGIA QTGELTVVLD GLVIEQENIT 150
NCSSSPTIEI QENGDALHEN GEPSARTTAR LAVEGINGID NHVPTSTLVQ 200
NSCCSYVVNG DNTPSSPSQV AARPKNTPAP KPLASEPADD TVNGESSSFA 250
PTDNASVTGT PVVSEENALS PNCTSTTVED PPVQEILTSS ENNECIPSTS 300
AELESEARSI LEPDTSNSRS SSAFEAAKSR QPDGCMDPVR QQSGNANTET 350
LPSGWEQRKD PHGRTYYVDH NTRTTTWERP QPLPPGWERR VDDRRRVYYV 400
DHNTRTTTWQ RPTMESVRNF EQWQSQRNQL QGAMQQFNQR YLYSASMLAA 450
ENDPYGPLPP GWEKRVDSTD RVYFVNHNTK TTQWEDPRTQ GLQNEEPLPE 500
GWEIRYTREG VRYFVDHNTR TTTFKDPRNG KSSVTKGGPQ IAYERGERWK 550
LAHFRYLCQS NALPSHVKIN VSRQTLFEDS FQQIMALKPY DLRRRLYVIF 600
RGEEGLDYGG LAREWFFLLS HEVLNPMYCL FEYAGKNNYC LQINPASTIN 650
PDHLSYFCFI GRFIAMALFH GKFIDTGFSL PFYKRMLSKK LTIKDLESID 700
TEFYNSLIWI RDNNIEECGL EMYFSVDMEI LGKVTSHDLK LGGSNILVTE 750
ENKDEYIGLM TEWRFSRGVQ EQTKAFLDGF NEVVPLQWLQ YEDEKELEVM 800
LCGMQEVDLA DWQRNTVYRH YTRNSKQIIW FWQFVKETDN EVRMRLLQFV 850
TGTCRLPLGG FAELMGSNGP QKFCIEKVGK DTWLPRSHTC FNRLDLPPYK 900
SYEQLKEKLL FAIEETEGFG QE 922
WW4 (496-529):
(SEQ ID NO: 5)
EPLPEGWEIRYTREGVRYFVDHNTRTTTFKDPRN.
WW1 (349-382):
(SEQ ID NO: 6)
ETLPSGWEQRKDPHGRTYYVDHNTRTTTWERPQP.
WW2 (381-414):
(SEQ ID NO: 7)
QPLPPGWERRVDDRRRVYYVDHNTRTTTWQRPTM.
WW3 (456-489):
(SEQ ID NO: 8)
ENDPYGPLPPGWEKRVDSTDRVYFVNHNTKTTQWEDPRT.

Human WWP2 amino acid sequence (uniprot.org/uniprot/000308). The four underlined WW domains correspond to amino acids 300-333 (WW1), 330-363 (WW2), 405-437 (WW3), and 444-547 (WW4).

(SEQ ID NO: 9)
MASASSSRAG VALPFEKSQL TLKVVSAKPK VHNRQPRINS YVEVAVDGLP 50
SETKKTGKRI GSSELLWNEI IILNVTAQSH LDLKVWSCHT LRNELLGTAS 100
VNLSNVLKNN GGKMENMQLT LNLQTENKGS VVSGGELTIF LDGPTVDLGN 150
VPNGSALTDG SQLPSRDSSG TAVAPENRHQ PPSINCEGGR SRTHRHSGAS 200
ARTTPATGEQ SPGARSRHRQ PVKNSGHSGL ANGTVNDEPT TATDPEEPSV 250
VGVTSPPAAP LSVTPNPNTT SLPAPATPAE GEEPSTSGTQ QLPAAAQAPD 300
ALPAGWEQRE LPNGRVYYVD HNTKTTTWER PLPPGWEKRT DPRGRFYYVD 350
HNTRTTTWQR PTAEYVRNYE QWQSQRNQLQ GAMQHFSQRF LYQSSSASTD 400
HDPLGPLPPG WEKRQDNGRV YYVNHNTRIT QWEDPRTQGM IQEPALPPGW 450
EMKYTSEGVR YFVDHNTRTT TFKDPRPGFE SGTKQGSPGA YDRSFRWKYH 500
QFRFLCHSNA LPSHVKISVS RQTLFEDSFQ QIMNMKPYDL RRRLYIIMRG 550
EEGLDYGGIA REWFELLSHE VLNPMYCLFE YAGKNNYCLQ INPASSINPD 600
HLTYFRFIGR FIAMALYHGK FIDTGFTLPF YKRMLNKRPT LKDLESIDPE 650
FYNSIVWIKE NNLEECGLEL YFIQDMEILG KVTTHELKEG GESIRVTEEN 700
KEEYIMLLTD WRFTRGVEEQ TKAFLDGFNE VAPLEWLRYF DEKELELMLC 750
GMQEIDMSDW QKSTIYRHYT KNSKQIQWFW QVVKEMDNEK RIRLLQFVTG 800
TCRLPVGGFA ELIGSNGPQK FCIDKVGKET WLPRSHTCEN RLDLPPYKSY 850
EQLREKLLYA IEETEGFGQE 870
WW1 (300-333):
(SEQ ID NO: 10)
DALPAGWEQRELPNGRVYYVDHNTKTTTWERPLP.
WW2 (330-363):
(SEQ ID NO: 11)
PLPPGWEKRT DPRGRFYYVDHNTRTTTWQRPTA.
WW3 (405-437):
(SEQ ID NO: 12)
HDPLGPLPPGWEKRQDNGRVYYVNHNTRTTQWEDPRT.
WW4 (444-477):
(SEQ ID NO: 13)
PALPPGWEMKYTSEGVRYFVDHNTRTTTFKDPRP.

Human Nedd4-1 amino acid sequence (uniprot.org/uniprot/P46934). The four underlined WW domains correspond to amino acids 610-643 (WW1), 767-800 (WW2), 840-873 (WW3), and 892-925 (WW4).

(SEQ ID NO: 14)
MAQSLRLHFA ARRSNTYPLS ETSGDDLDSH VHMCFKRPTR ISTSNVVQMK 50
LTPRQTALAP LIKENVQSQE RSSVPSSENV NKKSSCLQIS LQPTRYSGYL 100
QSSNVLADSD DASFTCILKD GIYSSAVVDN ELNAVNDGHL VSSPAICSGS 150
LSNFSTSDNG SYSSNGSDFG SCASITSGGS YTNSVISDSS SYTFPPSDDT 200
FLGGNLPSDS TSNRSVPNRN TTPCEIFSRS TSTDPFVQDD LEHGLEIMKL 250
PVSRNTKIPL KRYSSLVIFP RSPSTTRPTS PTSLCTLLSK GSYQTSHQFI 300
ISPSEIAHNE DGTSAKGFLS TAVNGLRLSK TICTPGEVRD IRPLHRKGSL 350
QKKIVLSNNT PRQTVCEKSS EGYSCVSVHF TQRKAATLDC ETTNGDCKPE 400
MSEIKLNSDS EYIKLMHRTS ACLPSSQNVD CQININGELE RPHSQMNKNH 450
GILRRSISLG GAYPNISCLS SLKHNCSKGG PSQLLIKFAS GNEGKVDNLS 500
RDSNRDCTNE LSNSCKTRDD FLGQVDVPLY PLPTENPRLE RPYTFKDFVL 550
HPRSHKSRVK GYLRLKMTYL PKTSGSEDDN AEQAEELEPG WVVLDQPDAA 600
CHLQQQQEPS PLPPGWEERQ DILGRTYYVN HESRRTQWKR PTPQDNLTDA 650
ENGNIQLQAQ RAFTTRRQIS EETESVDNRE SSENWEIIRE DEATMYSNQA 700
FPSPPPSSNL DVPTHLAEEL NARLTIFGNS AVSQPASSSN HSSRRGSLQA 750
YTFEEQPTLP VLLPTSSGLP PGWEEKQDER GRSYYVDHNS RTTTWTKPTV 800
QATVETSQLT SSQSSAGPQS QASTSDSGQQ VTQPSEIEQG FLPKGWEVRH 850
APNGRPFFID HNTKTTTWED PRLKIPAHLR GKTSLDTSND LGPLPPGWEE 900
RTHTDGRIFY INHNIKRTQW EDPRLENVAI TGPAVPYSRD YKRKYEFFRR 950
KLKKQNDIPN KFEMKLRRAT VLEDSYRRIM GVKRADFLKA RLWIEFDGEK 1000
GLDYGGVARE WFFLISKEMF NPYYGLFEYS ATDNYTLQIN PNSGLCNEDH 1050
LSYFKFIGRV AGMAVYHGKL LDGFFIRPFY KMMLHKPITL HDMESVDSEY 1100
YNSLRWILEN DPTELDLRFI IDEELFGQTH QHELKNGGSE IVVTNKNKKE 1150
YIYLVIQWRF VNRIQKQMAA FKEGFFELIP QDLIKIFDEN ELELLMCGLG 1200
DVDVNDWREH TKYKNGYSAN HQVIQWFWKA VLMMDSEKRI RLLQFVTGTS 1250
RVPMNGFAEL YGSNGPQSFT VEQWGTPEKL PRAHTCENRL DLPPYESFEE 1300
LWDKLQMAIE NTQGEDGVD 1319
WW1(610-643):
(SEQ ID NO: 15)
SPLPPGWEERQDILGRTYYVNHESRRTQWKRPTP.
WW2 (767-800):
(SEQ ID NO: 16)
SGLPPGWEEKQDERGRSYYVDHNSRTTTWTKPTV.
WW3 (840-873):
(SEQ ID NO: 17)
GFLPKGWEVRHAPNGRPFFIDHNTKTTTWEDPRL.
WW4 (892-925):
(SEQ ID NO: 18)
GPLPPGWEERTHTDGRIFYINHNIKRTQWEDPRL.

Human Nedd4-2 amino acid sequence (>gi|21361472|ref|NP_056092.2|E3 ubiquitin-protein ligase NEDD4-like isoform 3 [Homo sapiens]). The four underlined WW domains correspond to amino acids 198-224 (WW1), 368-396 (WW2), 480-510 (WW3), and 531-561 (WW4).

(SEQ ID NO: 19)
MATGLGEPVYGLSEDEGESRILRVKVVSGIDLAKKDIFGASDPYVKLSLYV
ADENRELALVQTKTIKKTLNPKWNEEFYFRVNPSNHRLLFEVEDENRLTR
DDFLGQVDVPLSHLPTEDPTMERPYTFKDFLLRPRSHKSRVKGFLRLKMA
YMPKNGGQDEENSDQRDDMEHGWEVVDSNDSASQHQEELPPPPLPPGWEE
KVDNLGRTYYVNHNNRTTQWHRPSLMDVSSESDNNIRQINQEAAHRRFRS
RRHISEDLEPEPSEGGDVPEPWETISEEVNIAGDSLGLALPPPPASPGSR
TSPQELSEELSRRLQITPDSNGEQFSSLIQREPSSRLRSCSVTDAVAEQG
HLPPPSVAYVHTTPGLPSGWEERKDAKGRTYYVNHNNRTTTWTRPIMQLA
EDGASGSATNSNNHLIEPQIRRPRSLSSPTVTLSAPLEGAKDSPVRRAVK
DTLSNPQSPQPSPYNSPKPQHKVTQSFLPPGWEMRIAPNGRPFFIDHNTK
TTTWEDPRLKFPVHMRSKTSLNPNDLGPLPPGWEERIHLDGRTFYIDHNS
KITQWEDPRLQNPAITGPAVPYSREFKQKYDYFRKKLKKPADIPNRFEMK
LHRNNIFEESYRRIMSVKRPDVLKARLWIEFESEKGLDYGGVAREWFFLL
SKEMENPYYGLFEYSATDNYTLQINPNSGLCNEDHLSYFTFIGRVAGLAV
FHGKLLDGFFIRPFYKMMLGKQITLNDMESVDSEYYNSLKWILENDPTEL
DLMFCIDEENFGQTYQVDLKPNGSEIMVTNENKREYIDLVIQWRFVNRVQ
KQMNAFLEGFTELLPIDLIKIFDENELELLMCGLGDVDVNDWRQHSIYKN
GYCPNHPVIQWFWKAVLLMDAEKRIRLLQFVTGTSRVPMNGFAELYGSNG
PQLFTIEQWGSPEKLPRAHTCENRLDLPPYETFEDLREKLLMAVENAQGF
EGVD
WW1(198-224):
(SEQ ID NO: 20)
GWEEKVDNLGRTYYVNHNNRTTQWHRP.
WW2 (368-396):
(SEQ ID NO: 21)
PSGWEERKDAKGRTYYVNHNNRTTTWTRP.
WW3 (480-510):
(SEQ ID NO: 22)
PPGWEMRIAPNGRPFFIDHNTKTTTWEDPRL.
WW4 (531-561):
(SEQ ID NO: 23)
PPGWEERIHLDGRTFYIDHNSKITQWEDPRL.

Human Smurf1 amino acid sequence (uniprot.org/uniprot/Q9HCE7). The two underlined WW domains correspond to amino acids 234-267 (WW1) and 306-339 (WW2).

(SEQ ID NO: 24)
MSNPGTRRNG SSIKIRLTVL CAKNLAKKDF FRLPDPFAKI VVDGSGQCHS 50
TDTVKNTLDP KWNQHYDLYV GKTDSITISV WNHKKIHKKQ GAGFLGCVRL 100
LSNAISRLKD TGYQRLDLCK LNPSDTDAVR GQIVVSLQTR DRIGTGGSV 150
DCRGLLENEG TVYEDSGPGR PLSCEMEEPA PYTDSTGAAA GGGNCRFVES 200
PSQDQRLQAQ RLRNPDVRGS LQTPQNRPHG HQSPELPEGY EQRTTVQGQV 250
YFLHTQTGVS TWHDPRIPSP SGTIPGGDAA FLYEFLLQGH TSEPRDLNSV 300
NCDELGPLPP GWEVRSTVSG RIYFVDHNNR TTQFTDPRLH HIMNHQCQLK 350
EPSQPLPLPS EGSLEDEELP AQRYERDLVQ KLKVLRHELS LQQPQAGHCR 400
IEVSREEIFE ESYRQIMKMR PKDLKKRLMV KERGEEGLDY GGVAREWLYL 450
LCHEMLNPYY GLFQYSTDNI YMLQINPDSS INPDHLSYFH FVGRIMGLAV 500
FHGHYINGGF TVPFYKQLLG KPIQLSDLES VDPELHKSLV WILENDITPV 550
LDHTFCVEHN AFGRILQHEL KPNGRNVPVT EENKKEYVRL YVNWREMRGI 600
EAQFLALQKG FNELIPQHLL KPFDQKELEL IIGGLDKIDL NDWKSNTRLK 650
HCVADSNIVR WEWQAVETED EERRARLLQF VTGSTRVPLQ GFKALQGSTG 700
AAGPRLFTIH LIDANTDNLP KAHTCENRID IPPYESYEKL YEKLLTAVEE 750
TCGFAVE 757
WW1 (234-267):
(SEQ ID NO: 25)
PELPEGYEQRTTVQGQVYFLHTQTGVSTWHDPRI.
WW2 (306-339):
(SEQ ID NO: 26)
GPLPPGWEVRSTVSGRIYFVDHNNRTTQFTDPRL.

Human Smurf2 amino acid sequence (uniprot.org/uniprot/Q9HAU4). The three underlined WW domains correspond to amino acids 157-190 (WW1), 251-284 (WW2), and 297-330 (WW3).

(SEQ ID NO: 27)
MSNPGGRRNG PVKLRLTVLC AKNLVKKDFF RLPDPFAKVV VDGSGQCHST 50
DTVKNTLDPK WNQHYDLYIG KSDSVTISVW NHKKIHKKQG AGFLGCVRLL 100
SNAINRLKDT GYQRLDLCKL GPNDNDTVRG QIVVSLQSRD RIGTGGQVVD 150
CSRLFDNDLP DGWEERRTAS GRIQYLNHIT RTTQWERPTR PASEYSSPGR 200
PLSCFVDENT PISGINGATC GQSSDPRLAE RRVRSQRHRN YMSRTHLHTP 250
PDLPEGYEQR TTQQGQVYFL HTQTGVSTWH DPRVPRDLSN INCEELGPLP 300
PGWEIRNTAT GRVYFVDHNN RTTQFTDPRL SANLHLVLNR QNQLKDQQQQ 350
QVVSLCPDDT ECLTVPRYKR DLVQKLKILR QELSQQQPQA GHCRIEVSRE 400
EIFEESYRQV MKMRPKDLWK RLMIKFRGEE GLDYGGVARE WLYLLSHEML 450
NPYYGLFQYS RDDIYTLQIN PDSAVNPEHL SYFHFVGRIM GMAVFHGHYI 500
DGGFTLPFYK QLLGKSITLD DMELVDPDLH NSLVWILEND ITGVLDHTFC 550
VEHNAYGEII QHELKPNGKS IPVNEENKKE YVRLYVNWRF LRGIEAQFLA 600
LQKGENEVIP QHLLKTEDEK ELELIICGLG KIDVNDWKVN TRLKHCTPDS 650
NIVKWFWKAV EFFDEERRAR LLQFVTGSSR VPLQGFKALQ GAAGPRLFTI 700
HQIDACTNNL PKAHTCENRI DIPPYESYEK LYEKLLTAIE ETCGFAVE 748
WW1 (157-190):
(SEQ ID NO: 28)
NDLPDGWEERRTASGRIQYLNHITRTTQWERPTR.
WW2 (251-284):
(SEQ ID NO: 29)
PDLPEGYEQRTTQQGQVYFLHTQTGVSTWHDPRV.
WW3 (297-330):
(SEQ ID NO: 30)
GPLPPGWEIRNTATGRVYFVDHNNRTTQFTDPRL.

Human ITCH amino acid sequence (uniprot.org/uniprot/Q96J02). The four underlined WW domains correspond to amino acids 326-359 (WW1), 358-391 (WW2), 438-471 (WW3), and 478-511 (WW4).

(SEQ ID NO: 31)
MSDSGSQLGS MGSLTMKSQL QITVISAKLK ENKKNWFGPS PYVEVTVDGQ 50
SKKTEKCNNT NSPKWKQPLT VIVTPVSKLH FRVWSHQTLK SDVLLGTAAL 100
DIYETLKSNN MKLEEVVVTL QLGGDKEPTE TIGDLSICLD GLQLESEVVT 150
NGETTCSENG VSLCLPRLEC NSAISAHCNL CLPGLSDSPI SASRVAGFTG 200
ASQNDDGSRS KDETRVSING SDDPEDAGAG ENRRVSGNNS PSLSNGGFKP 250
SRPPRPSRPP PPTPRRPASV NGSPSATSES DGSSTGSLPP TNTNTNTSEG 300
ATSGLIIPLT ISGGSGPRPL NPVTQAPLPP GWEQRVDQHG RVYYVDHVEK 350
RTTWDRPEPL PPGWERRVDN MGRIYYVDHF TRTTTWQRPT LESVRNYEQW 400
QLQRSQLQGA MQQFNQRFIY GNQDLFATSQ SKEFDPLGPL PPGWEKRTDS 450
NGRVYFVNHN TRITQWEDPR SQGQLNEKPL PEGWEMRFTV DGIPYFVDHN 500
RRTTTYIDPR TGKSALDNGP QIAYVRDFKA KVQYFRFWCQ QLAMPQHIKI 550
TVTRKTLFED SFQQIMSESP QDLRRRLWVI FPGEEGLDYG GVAREWFFLL 600
SHEVLNPMYC LFEYAGKDNY CLQINPASYI NPDHLKYFRF IGRFIAMALF 650
HGKFIDTGES LPFYKRILNK PVGLKDLESI DPEFYNSLIW VKENNIEECD 700
LEMYFSVDKE ILGEIKSHDL KPNGGNILVT EENKEEYIRM VAEWRLSRGV 750
EEQTQAFFEG FNEILPQQYL QYFDAKELEV LLCGMQEIDL NDWQRHAIYR 800
HYARTSKQIM WFWQFVKEID NEKRMRLLQF VTGTCRLPVG GFADLMGSNG 850
PQKFCIEKVG KENWLPRSHT CFNRLDLPPY KSYEQLKEKL LFAIEETEGF 900
GQE 903
ITCH WW1 (326-359):
(SEQ ID NO: 32)
APLPPGWEQRVDQHGRVYYVDHVEKRTTWDRPEP.
ITCH WW2 (358-391):
(SEQ ID NO: 33)
EPLPPGWERRVDNMGRIYYVDHFTRITTWQRPTL.
ITCH WW3 (438-471):
(SEQ ID NO: 34)
GPLPPGWEKRTDSNGRVYFVNHNTRITQWEDPRS.
ITCH WW4 (478-511):
(SEQ ID NO: 35)
KPLPEGWEMRFTVDGIPYFVDHNRRTTTYIDPRT.

Human NEDL1 amino acid sequence (uniprot.org/uniprot/Q76N89). The two underlined WW domains correspond to amino acids 829-862 (WW1), and 1018-1051 (WW2).

(SEQ ID NO: 36)
MLLHLCSVKN LYQNRFLGLA AMASPSRNSQ SRRRCKEPLR YSYNPDQFHN 50
MDLRGGPHDG VTIPRSTSDT DLVTSDSRST LMVSSSYYSI GHSQDLVIHW 100
DIKEEVDAGD WIGMYLIDEV LSENFLDYKN RGVNGSHRGQ IIWKIDASSY 150
FVEPETKICF KYYHGVSGAL RATTPSVTVK NSAAPIFKSI GADETVQGQG 200
SRRLISESLS DFQAMGLKKG MFFNPDPYLK ISIQPGKHSI FPALPHHGQE 250
RRSKIIGNTV NPIWQAEQFS FVSLPTDVLE IEVKDKFAKS RPIIKRFLGK 300
LSMPVQRLLE RHAIGDRVVS YTLGRRLPTD HVSGQLQFRF EITSSIHPDD 350
EEISLSTEPE SAQIQDSPMN NLMESGSGEP RSEAPESSES WKPEQLGEGS 400
VPDGPGNQSI ELSRPAEEAA VITEAGDQGM VSVGPEGAGE LLAQVQKDIQ 450
PAPSAEELAE QLDLGEEASA LLLEDGEAPA STKEEPLEEE ATTQSRAGRE 500
EEEKEQEEEG DVSTLEQGEG RLQLRASVKR KSRPCSLPVS ELETVIASAC 550
GDPETPRTHY IRIHTLLHSM PSAQGGSAAE EEDGAEEEST LKDSSEKDGL 600
SEVDTVAADP SALEEDREEP EGATPGTAHP GHSGGHFPSL ANGAAQDGDT 650
HPSTGSESDS SPRQGGDHSC EGCDASCCSP SCYSSSCYST SCYSSSCYSA 700
SCYSPSCYNG NRFASHTRES SVDSAKISES TVESSQDDEE EENSAFESVP 750
DSMQSPELDP ESTNGAGPWQ DELAAPSGHV ERSPEGLESP VAGPSNRREG 800
ECPILHNSQP VSQLPSLRPE HHHYPTIDEP LPPNWEARID SHGRVFYVDH 850
VNRTTTWQRP TAAATPDGMR RSGSIQQMEQ LNRRYQNIQR TIATERSEED 900
SGSQSCEQAP AGGGGGGGSD SEAESSQSSL DLRREGSLSP VNSQKITLLL 950
QSPAVKFITN PEFFTVLHAN YSAYRVFTSS TCLKHMILKV RRDARNFERY 1000
QHNRDLVNFI NMFADTRLEL PRGWEIKTDQ QGKSFFVDHN SRATTFIDPR 1050
IPLQNGRLPN HLTHRQHLQR LRSYSAGEAS EVSRNRGASL LARPGHSLVA 1100
AIRSQHQHES LPLAYNDKIV AFLRQPNIFE MLQERQPSLA RNHTLREKIH 1150
YIRTEGNHGL EKLSCDADLV ILLSLFEEEI MSYVPLQAAF HPGYSESPRC 1200
SPCSSPQNSP GLQRASARAP SPYRRDFEAK LRNFYRKLEA KGFGQGPGKI 1250
KLIIRRDHLL EGTFNQVMAY SRKELQRNKL YVTFVGEEGL DYSGPSREFF 1300
FLLSQELFNP YYGLFEYSAN DTYTVQISPM SAFVENHLEW FRESGRILGL 1350
ALIHQYLLDA FFTRPFYKAL LRLPCDLSDL EYLDEEFHQS LQWMKDNNIT 1400
DILDLTFTVN EEVFGQVTER ELKSGGANTQ VTEKNKKEYI ERMVKWRVER 1450
GVVQQTEALV RGFYEVVDSR LVSVEDAREL ELVIAGTAEI DLNDWRNNTE 1500
YRGGYHDGHL VIRWFWAAVE RENNEQRLRL LQFVTGTSSV PYEGFAALRG 1550
SNGLRRFCIE KWGKITSLPR AHTCENRLDL PPYPSYSMLY EKLLTAVEET 1600
STFGLE 1606
WW1 (829-862):
(SEQ ID NO: 37)
PLPPNWEARIDSHGRVFYVDHVNRTTTWQRPTA.
WW2 (1018-1051):
(SEQ ID NO: 38)
LELPRGWEIKTDQQGKSFFVDHNSRATTFIDPRI.

Human NEDL2 amino acid sequence (uniprot.org/uniprot/Q9P2P5). The two underlined WW domains correspond to amino acids 807-840 (WW1) and 985-1018 (WW2).

(SEQ ID NO: 39)
MASSAREHLL FVRRRNPQMR YTLSPENLQS LAAQSSMPEN MTLQRANSDT 50
DLVTSESRSS LTASMYEYTL GQAQNLIIFW DIKEEVDPSD WIGLYHIDEN 100
SPANFWDSKN RGVTGTQKGQ IVWRIEPGPY FMEPEIKICF KYYHGISGAL 150
RATTPCITVK NPAVMMGAEG MEGGASGNLH SRKLVSFTLS DLRAVGLKKG 200
MFFNPDPYLK MSIQPGKKSS FPTCAHHGQE RRSTIISNTT NPIWHREKYS 250
FFALLTDVLE IEIKDKFAKS RPIIKRELGK LTIPVQRLLE RQAIGDQMLS 300
YNLGRRLPAD HVSGYLQFKV EVTSSVHEDA SPEAVGTILG VNSVNGDLGS 350
PSDDEDMPGS HHDSQVCSNG PVSEDSAADG TPKHSFRISS TLEIDTEELT 400
STSSRTSPPR GRQDSLNDYL DAIEHNGHSR PGTATCSERS MGASPKLRSS 450
FPTDTRLNAM LHIDSDEEDH EFQQDLGYPS SLEEEGGLIM FSRASRADDG 500
SLTSQTKLED NPVENEEAST HEAASFEDKP ENLPELAESS LPAGPAPEEG 550
EGGPEPQPSA DQGSAELCGS QEVDQPTSGA DTGTSDASGG SRRAVSETES 600
LDQGSEPSQV SSETEPSDPA RTESVSEAST RPEGESDLEC ADSSCNESVT 650
TQLSSVDTRC SSLESARFPE TPAFSSQEEE DGACAAEPTS SGPAEGSQES 700
VCTAGSLPVV QVPSGEDEGP GAESATVPDQ EELGEVWQRR GSLEGAAAAA 750
ESPPQEEGSA GEAQGTCEGA TAQEEGATGG SQANGHQPLR SLPSVRQDVS 800
RYQRVDEALP PNWEARIDSH GRIFYVDHVN RTTTWQRPTA PPAPQVLQRS 850
NSIQQMEQLN RRYQSIRRTM TNERPEENTN AIDGAGEEAD FHQASADERR 900
ENILPHSTSR SRITLLLQSP PVKFLISPEF FTVLHSNPSA YRMFTNNTCL 950
KHMITKVRRD THHFERYQHN RDLVGELNMF ANKQLELPRG WEMKHDHQGK 1000
AFFVDHNSRT TTFIDPRLPL QSSRPTSALV HRQHLTRQRS HSAGEVGEDS 1050
RHAGPPVLPR PSSTENTVSR PQYQDMVPVA YNDKIVAFLR QPNIFEILQE 1100
RQPDLTRNHS LREKIQFIRT EGTPGLVRLS SDADLVMLLS LFEEEIMSYV 1150
PPHALLHPSY CQSPRGSPVS SPQNSPGTQR ANARAPAPYK RDFEAKLRNF 1200
YRKLETKGYG QGPGKLKLII RRDHLLEDAF NQIMGYSRKD LQRNKLYVTF 1250
VGEEGLDYSG PSREFFFLVS RELFNPYYGL FEYSANDTYT VQISPMSAFV 1300
DNHHEWFRES GRILGLALIH QYLLDAFFTR PFYKALLRIL CDLSDLEYLD 1350
EEFHQSLQWM KDNDIHDILD LTFTVNEEVF GQITERELKP GGANIPVTEK 1400
NKKEYIERMV KWRIERGVVQ QTESLVRGFY EVVDARLVSV FDARELELVI 1450
AGTAEIDLSD WRNNTEYRGG YHDNHIVIRW FWAAVERENN EQRLRLLQFV 1500
TGTSSIPYEG FASLRGSNGP RRFCVEKWGK ITALPRAHTC FNRLDLPPYP 1550
SFSMLYEKLL TAVEETSTEG LE 1572
WW1 (807-840):
(SEQ ID NO: 40)
EALPPNWEARIDSHGRIFYVDHVNRTTTWQRPTA.
WW2 (985-1018):
(SEQ ID NO: 41)
LELPRGWEMKHDHQGKAFFVDHNSRTTTFIDPRL.

In some embodiments, the WW domain consists essentially of a WW domain or WW domain variant. Consists essentially of means that a domain, peptide, or polypeptide consists essentially of an amino acid sequence when such an amino acid sequence is present with only a few additional amino acid residues, for example, from about 1 to about 10 or so additional residues, typically from 1 to about 5 additional residues in the domain, peptide, or polypeptide.

Alternatively, the WW domain may be a WW domain that has been modified to include two basic amino acids at the C-terminus of the domain. Techniques are known in the art and are described in the art, for example, in Sambrook et al., Molecular Cloning: a Laboratory Manual, 3rd ed., Cold Spring Harbor Laboratory Press (2001). Thus, a skilled person could readily modify an existing WW domain that does not normally have two C-terminal basic residues so as to include two basic residues at the C-terminus.

Basic amino acids are amino acids that possess a side-chain functional group that has a pKa of greater than 7 and includes lysine, arginine, and histidine, as well as basic amino acids that are not included in the twenty α-amino acids commonly included in proteins. The two basic amino acids at the C-terminus of the WW domain may be the same basic amino acid or different basic amino acids. In one embodiment, the two basic amino acids are two arginines.

The term WW domain also includes variants of a WW domain provided that any such variant possesses two basic amino acids at its C-terminus and maintains the ability of the WW domain to associate with the PPXY (SEQ ID NO: 2) motif A variant of such a WW domain refers to a WW domain that retains the ability of the variant to associate with the PPXY (SEQ ID NO: 2) motif (i.e., the PPXY (SEQ ID NO:2) motif of ARRDC1 and that has been mutated at one or more amino acids, including point, insertion, and/or deletion mutations, but still retains the ability to associate with the PPXY (SEQ ID NO: 2) motif A variant or derivative, therefore, includes deletions, including truncations and fragments; insertions and additions, for example, conservative substitutions, site-directed mutants and allelic variants; and modifications, including one or more non-amino acyl groups (e.g., sugar, lipid, etc.) covalently linked to the peptide and post-translational modifications. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing.

The WW domain may be part of a longer protein. Thus, the protein, in various different embodiments, comprises the WW domain, consists of the WW domain, or consists essentially of the WW domain, as defined herein. The polypeptide may be a protein that includes a WW domain as a functional domain within the protein sequence.

The term “Cas9,” or “Cas9 protein,” or “Cas9 polypeptide” refers to an RNA-guided nuclease comprising a Cas9 protein as well as fusion proteins containing such Cas9 proteins and variants thereof (e.g., a protein comprising an active, inactive, or altered DNA cleavage domain of Cas9, and/or the gRNA binding domain of Cas9). In some embodiments, the fused proteins may include those that modify the epigenome or control transcriptional activity. The variants may include deletions or additions, such as e.g., addition of one, two, or more nuclear localization sequences (such as from SV40 and others known in the art), e.g., 1, 2, 3, 4, 5, 6, 7, 8, 9, 10 such sequences or a range between and including any two of the foregoing values.

In some embodiments, the Cas9 polypeptide is a Cas9 protein found in a type II CRISPR-associated system. Suitable Cas9 polypeptides that may be used in certain embodiments of the present invention include, but are not limited to, Cas9 protein from Streptococcus pyogenes (Sp. Cas9), F. novicida, S. aureus, S, thermophiles, N. meningitidis, and variants thereof.

A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (e.g., viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (mc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature. DNA-binding and cleavage typically requires protein and both RNAs. However, single guide RNAs (“sgRNA,”, or simply “gRNA”) can be engineered so as to incorporate aspects of both the crRNA and tracrRNA into a single RNA species. (See. e.g., Jinek M., et al., Science, 337:816-821 (2012), the entire contents of which is hereby incorporated by reference). Cas9 recognizes a short motif in the CRISPR repeat sequences (the PAM or protospacer adjacent motif) to help distinguish self versus non-self. Cas9 nuclease sequences and structures are well known to those of skill in the art. (See, e.g., Ferretti et al., “Complete genome sequence of an M1 strain of Streptococcus pyogenes,” Proc. Natl. Acad. Sci. U.S.A., 98:4658-4663 (2001); “CRISPR RNA maturation by trans-encoded small RNA and host factor RNase III,” Deltcheva E., et al., “CRISPR RNA maturation by trans-encoded small RNA and host factor RNase III,” Nature. 471:602-607 (2011); and Jinek, M., et al., “A programmable dual-RNA-guided DNA endonuclease in adaptive bacterial immunity,” Science. 337:816-821 (2012), the entire contents of each of which are incorporated herein by reference). Additional suitable Cas9 nucleases and sequences will be apparent to those of skill in the art based on this disclosure, and such Cas9 nucleases and sequences include Cas9 sequences from the organisms and loci disclosed in the art. (See, e.g., Chylinski, Rhun, and Charpentier, “The tracrRNA and Cas9 families of type II CRISPR-Cas immunity systems,” RNA Biology, 10:5, 726-737 (2013); Karvelis, G., et al., “Harnessing the natural diversity and in vitro evolution of Cas9 to expand the genome editing toolbox,” Current Opinion in Microbiology. 37:88-94 (2017); Komor, A. C., et al., “CRISPR-Based Technologies for the Manipulation of Eukaryotic Genomes,” Cell. 168:20-36 (2017); and Murovec, J., et al., “New variants of CRISPR RNA-guided genome editing enzymes,” Plant Biotechnol. J., 15:917-26 (2017), the entire contents of each of which are incorporated herein by reference).

In some embodiments, the Cas9 polypeptide is a wild-type Cas9, a nickase, or comprises a nuclease inactivated (“dCas9” for nuclease-“dead” Cas9) protein.

Methods for generating a Cas9 protein (or a variant thereof) having an inactive DNA cleavage domain are known (See, e.g., Jinek et al., Science. 337:816-821(2012); Qi et al., “Repurposing CRISPR as an RNA-Guided Platform for Sequence-Specific Control of Gene Expression,” Cell, 28:152(5):1173-83 (2013), the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H841A completely inactivate the nuclease activity of S. pyogenes Cas9 (Jinek et al., Science, 337:816-821 (2012); Qi et al., Cell, 28; 152(5):1173-83 (2013)). In some embodiments, proteins comprising variants of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or variants thereof are referred to as “Cas9 variants.” A Cas9 variant shares homology to Cas9, or a variant thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to wild type Cas9. In some embodiments, the Cas9 variant comprises a variant of Cas9 (e.g., a gRNA binding domain or a DNA-cleavage domain), such that the variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to the corresponding variant of wild type Cas9. In some embodiments, wild type Cas9 corresponds to Cas9 from Streptococcus pyogenes (NCBI Reference Sequence: NC_017053.1, SEQ ID NO:1 (nucleotide); SEQ ID NO:2 (amino acid)).

(SEQ ID NO: 1)
ATGGATAAGAAATACTCAATAGGCTTAGATATCGGCACAAATAGCGTCGGATGGGCGGTGAT
CACTGATGATTATAAGGTTCCGTCTAAAAAGTTCAAGGTTCTGGGAAATACAGACCGCCACA
GTATCAAAAAAAATCTTATAGGGGCTCTTTTATTTGGCAGTGGAGAGACAGCGGAAGCGACT
CGTCTCAAACGGACAGCTCGTAGAAGGTATACACGTCGGAAGAATCGTATTTGTTATCTACA
GGAGATTTTTTCAAATGAGATGGCGAAAGTAGATGATAGTTTCTTTCATCGACTTGAAGAGT
CTTTTTTGGTGGAAGAAGACAAGAAGCATGAACGTCATCCTATTTTTGGAAATATAGTAGAT
GAAGTTGCTTATCATGAGAAATATCCAACTATCTATCATCTGCGAAAAAAATTGGCAGATTC
TACTGATAAAGCGGATTTGCGCTTAATCTATTTGGCCTTAGCGCATATGATTAAGTTTCGTG
GTCATTTTTTGATTGAGGGAGATTTAAATCCTGATAATAGTGATGTGGACAAACTATTTATC
CAGTTGGTACAAATCTACAATCAATTATTTGAAGAAAACCCTATTAACGCAAGTAGAGTAGA
TGCTAAAGCGATTCTTTCTGCACGATTGAGTAAATCAAGACGATTAGAAAATCTCATTGCTC
AGCTCCCCGGTGAGAAGAGAAATGGCTTGTTTGGGAATCTCATTGCTTTGTCATTGGGATTG
ACCCCTAATTTTAAATCAAATTTTGATTTGGCAGAAGATGCTAAATTACAGCTTTCAAAAGA
TACTTACGATGATGATTTAGATAATTTATTGGCGCAAATTGGAGATCAATATGCTGATTTGT
TTTTGGCAGCTAAGAATTTATCAGATGCTATTTTACTTTCAGATATCCTAAGAGTAAATAGT
GAAATAACTAAGGCTCCCCTATCAGCTTCAATGATTAAGCGCTACGATGAACATCATCAAGA
CTTGACTCTTTTAAAAGCTTTAGTTCGACAACAACTTCCAGAAAAGTATAAAGAAATCTTTT
TTGATCAATCAAAAAACGGATATGCAGGTTATATTGATGGGGGAGCTAGCCAAGAAGAATTT
TATAAATTTATCAAACCAATTTTAGAAAAAATGGATGGTACTGAGGAATTATTGGTGAAACT
AAATCGTGAAGATTTGCTGCGCAAGCAACGGACCTTTGACAACGGCTCTATTCCCCATCAAA
TTCACTTGGGTGAGCTGCATGCTATTTTGAGAAGACAAGAAGACTTTTATCCATTTTTAAAA
GACAATCGTGAGAAGATTGAAAAAATCTTGACTTTTCGAATTCCTTATTATGTTGGTCCATT
GGCGCGTGGCAATAGTCGTTTTGCATGGATGACTCGGAAGTCTGAAGAAACAATTACCCCAT
GGAATTTTGAAGAAGTTGTCGATAAAGGTGCTTCAGCTCAATCATTTATTGAACGCATGACA
AACTTTGATAAAAATCTTCCAAATGAAAAAGTACTACCAAAACATAGTTTGCTTTATGAGTA
TTTTACGGTTTATAACGAATTGACAAAGGTCAAATATGTTACTGAGGGAATGCGAAAACCAG
CATTTCTTTCAGGTGAACAGAAGAAAGCCATTGTTGATTTACTCTTCAAAACAAATCGAAAA
GTAACCGTTAAGCAATTAAAAGAAGATTATTTCAAAAAAATAGAATGTTTTGATAGTGTTGA
AATTTCAGGAGTTGAAGATAGATTTAATGCTTCATTAGGCGCCTACCATGATTTGCTAAAAA
TTATTAAAGATAAAGATTTTTTGGATAATGAAGAAAATGAAGATATCTTAGAGGATATTGTT
TTAACATTGACCTTATTTGAAGATAGGGGGATGATTGAGGAAAGACTTAAAACATATGCTCA
CCTCTTTGATGATAAGGTGATGAAACAGCTTAAACGTCGCCGTTATACTGGTTGGGGACGTT
TGTCTCGAAAATTGATTAATGGTATTAGGGATAAGCAATCTGGCAAAACAATATTAGATTTT
TTGAAATCAGATGGTTTTGCCAATCGCAATTTTATGCAGCTGATCCATGATGATAGTTTGAC
ATTTAAAGAAGATATTCAAAAAGCACAGGTGTCTGGACAAGGCCATAGTTTACATGAACAGA
TTGCTAACTTAGCTGGCAGTCCTGCTATTAAAAAAGGTATTTTACAGACTGTAAAAATTGTT
GATGAACTGGTCAAAGTAATGGGGCATAAGCCAGAAAATATCGTTATTGAAATGGCACGTGA
AAATCAGACAACTCAAAAGGGCCAGAAAAATTCGCGAGAGCGTATGAAACGAATCGAAGAAG
GTATCAAAGAATTAGGAAGTCAGATTCTTAAAGAGCATCCTGTTGAAAATACTCAATTGCAA
AATGAAAAGCTCTATCTCTATTATCTACAAAATGGAAGAGACATGTATGTGGACCAAGAATT
AGATATTAATCGTTTAAGTGATTATGATGTCGATCACATTGTTCCACAAAGTTTCATTAAAG
ACGATTCAATAGACAATAAGGTACTAACGCGTTCTGATAAAAATCGTGGTAAATCGGATAAC
GTTCCAAGTGAAGAAGTAGTCAAAAAGATGAAAAACTATTGGAGACAACTTCTAAACGCCAA
GTTAATCACTCAACGTAAGTTTGATAATTTAACGAAAGCTGAACGTGGAGGTTTGAGTGAAC
TTGATAAAGCTGGTTTTATCAAACGCCAATTGGTTGAAACTCGCCAAATCACTAAGCATGTG
GCACAAATTTTGGATAGTCGCATGAATACTAAATACGATGAAAATGATAAACTTATTCGAGA
GGTTAAAGTGATTACCTTAAAATCTAAATTAGTTTCTGACTTCCGAAAAGATTTCCAATTCT
ATAAAGTACGTGAGATTAACAATTACCATCATGCCCATGATGCGTATCTAAATGCCGTCGTT
GGAACTGCTTTGATTAAGAAATATCCAAAACTTGAATCGGAGTTTGTCTATGGTGATTATAA
AGTTTATGATGTTCGTAAAATGATTGCTAAGTCTGAGCAAGAAATAGGCAAAGCAACCGCAA
AATATTTCTTTTACTCTAATATCATGAACTTCTTCAAAACAGAAATTACACTTGCAAATGGA
GAGATTCGCAAACGCCCTCTAATCGAAACTAATGGGGAAACTGGAGAAATTGTCTGGGATAA
AGGGCGAGATTTTGCCACAGTGCGCAAAGTATTGTCCATGCCCCAAGTCAATATTGTCAAGA
AAACAGAAGTACAGACAGGCGGATTCTCCAAGGAGTCAATTTTACCAAAAAGAAATTCGGAC
AAGCTTATTGCTCGTAAAAAAGACTGGGATCCAAAAAAATATGGTGGTTTTGATAGTCCAAC
GGTAGCTTATTCAGTCCTAGTGGTTGCTAAGGTGGAAAAAGGGAAATCGAAGAAGTTAAAAT
CCGTTAAAGAGTTACTAGGGATCACAATTATGGAAAGAAGTTCCTTTGAAAAAAATCCGATT
GACTTTTTAGAAGCTAAAGGATATAAGGAAGTTAAAAAAGACTTAATCATTAAACTACCTAA
ATATAGTCTTTTTGAGTTAGAAAACGGTCGTAAACGGATGCTGGCTAGTGCCGGAGAATTAC
AAAAAGGAAATGAGCTGGCTCTGCCAAGCAAATATGTGAATTTTTTATATTTAGCTAGTCAT
TATGAAAAGTTGAAGGGTAGTCCAGAAGATAACGAACAAAAACAATTGTTTGTGGAGCAGCA
TAAGCATTATTTAGATGAGATTATTGAGCAAATCAGTGAATTTTCTAAGCGTGTTATTTTAG
CAGATGCCAATTTAGATAAAGTTCTTAGTGCATATAACAAACATAGAGACAAACCAATACGT
GAACAAGCAGAAAATATTATTCATTTATTTACGTTGACGAATCTTGGAGCTCCCGCTGCTTT
TAAATATTTTGATACAACAATTGATCGTAAACGATATACGTCTACAAAAGAAGTTTTAGATG
CCACTCTTATCCATCAATCCATCACTGGTCTTTATGAAACACGCATTGATTTGAGTCAGCTA
GGAGGTGACTGA
(SEQ ID NO: 2)
MDKKYSIGLDIGTNSVGWAVITDDYKVPSKKFKVLGNTDRHSIKKNLIGALLFGSGETAEAT
RLKRTARRRYTRRKNRICYLQEIFSNEMAKVDDSFFHRLEESFLVEEDKKHERHPIFGNIVD
EVAYHEKYPTIYHLRKKLADSTDKADLRLIYLALAHMIKERGHFLIEGDLNPDNSDVDKLFI
QLVQIYNQLFEENPINASRVDAKAILSARLSKSRRLENLIAQLPGEKRNGLFGNLIALSLGL
TPNFKSNFDLAEDAKLQLSKDTYDDDLDNLLAQIGDQYADLFLAAKNLSDAILLSDILRVNS
EITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPELK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNRK
VTVKQLKEDYFKKIECFDSVEISGVEDRENASLGAYHDLLKIIKDKDFLDNEENEDILEDIV
LTLTLFEDRGMIEERLKTYAHLEDDKVMKQLKRRRYTGWGRLSRKLINGIRDKQSGKTILDE
LKSDGFANRNFMQLIHDDSLTFKEDIQKAQVSGQGHSLHEQIANLAGSPAIKKGILQTVKIV
DELVKVMGHKPENIVIEMARENQTTQKGQKNSRERMKRIEEGIKELGSQILKEHPVENTQLQ
NEKLYLYYLQNGRDMYVDQELDINRLSDYDVDHIVPQSFIKDDSIDNKVLTRSDKNRGKSDN
VPSEEVVKKMKNYWRQLLNAKLITQRKEDNLTKAERGGLSELDKAGFIKRQLVETRQITKHV
AQILDSRMNTKYDENDKLIREVKVITLKSKLVSDFRKDFQFYKVREINNYHHAHDAYLNAVV
GTALIKKYPKLESEFVYGDYKVYDVRKMIAKSEQEIGKATAKYFFYSNIMNFFKTEITLANG
EIRKRPLIETNGETGEIVWDKGRDFATVRKVLSMPQVNIVKKTEVQTGGFSKESILPKRNSD
KLIARKKDWDPKKYGGFDSPTVAYSVLVVAKVEKGKSKKLKSVKELLGITIMERSSFEKNPI
DFLEAKGYKEVKKDLIIKLPKYSLFELENGRKRMLASAGELQKGNELALPSKYVNFLYLASH
YEKLKGSPEDNEQKQLFVEQHKHYLDEIIEQISEFSKRVILADANLDKVLSAYNKHRDKPIR
EQAENIIHLFTLTNLGAPAAFKYFDTTIDRKRYTSTKEVLDATLIHQSITGLYETRIDLSQL
GGD (single underline: HNH domain; double underline: RuvC domain)

In some embodiments, wild type Cas9 corresponds to, or comprises SEQ ID NO:3 (nucleotide) and/or SEQ ID NO: 4 (amino acid):

(SEQ ID NO: 3)
ATGGATAAAAAGTATTCTATTGGTTTAGACATCGGCACTAATTCCGTTGGATGGGCTGTCAT
AACCGATGAATACAAAGTACCTTCAAAGAAATTTAAGGTGTTGGGGAACACAGACCGTCATT
CGATTAAAAAGAATCTTATCGGTGCCCTCCTATTCGATAGTGGCGAAACGGCAGAGGCGACT
CGCCTGAAACGAACCGCTCGGAGAAGGTATACACGTCGCAAGAACCGAATATGTTACTTACA
AGAAATTTTTAGCAATGAGATGGCCAAAGTTGACGATTCTTTCTTTCACCGTTTGGAAGAGT
CCTTCCTTGTCGAAGAGGACAAGAAACATGAACGGCACCCCATCTTTGGAAACATAGTAGAT
GAGGTGGCATATCATGAAAAGTACCCAACGATTTATCACCTCAGAAAAAAGCTAGTTGACTC
AACTGATAAAGCGGACCTGAGGTTAATCTACTTGGCTCTTGCCCATATGATAAAGTTCCGTG
GGCACTTTCTCATTGAGGGTGATCTAAATCCGGACAACTCGGATGTCGACAAACTGTTCATC
CAGTTAGTACAAACCTATAATCAGTTGTTTGAAGAGAACCCTATAAATGCAAGTGGCGTGGA
TGCGAAGGCTATTCTTAGCGCCCGCCTCTCTAAATCCCGACGGCTAGAAAACCTGATCGCAC
AATTACCCGGAGAGAAGAAAAATGGGTTGTTCGGTAACCTTATAGCGCTCTCACTAGGCCTG
ACACCAAATTTTAAGTCGAACTTCGACTTAGCTGAAGATGCCAAATTGCAGCTTAGTAAGGA
CACGTACGATGACGATCTCGACAATCTACTGGCACAAATTGGAGATCAGTATGCGGACTTAT
TTTTGGCTGCCAAAAACCTTAGCGATGCAATCCTCCTATCTGACATACTGAGAGTTAATACT
GAGATTACCAAGGCGCCGTTATCCGCTTCAATGATCAAAAGGTACGATGAACATCACCAAGA
CTTGACACTTCTCAAGGCCCTAGTCCGTCAGCAACTGCCTGAGAAATATAAGGAAATATTCT
TTGATCAGTCGAAAAACGGGTACGCAGGTTATATTGACGGCGGAGCGAGTCAAGAGGAATTC
TACAAGTTTATCAAACCCATATTAGAGAAGATGGATGGGACGGAAGAGTTGCTTGTAAAACT
CAATCGCGAAGATCTACTGCGAAAGCAGCGGACTTTCGACAACGGTAGCATTCCACATCAAA
TCCACTTAGGCGAATTGCATGCTATACTTAGAAGGCAGGAGGATTTTTATCCGTTCCTCAAA
GACAATCGTGAAAAGATTGAGAAAATCCTAACCTTTCGCATACCTTACTATGTGGGACCCCT
GGCCCGAGGGAACTCTCGGTTCGCATGGATGACAAGAAAGTCCGAAGAAACGATTACTCCAT
GGAATTTTGAGGAAGTTGTCGATAAAGGTGCGTCAGCTCAATCGTTCATCGAGAGGATGACC
AACTTTGACAAGAATTTACCGAACGAAAAAGTATTGCCTAAGCACAGTTTACTTTACGAGTA
TTTCACAGTGTACAATGAACTCACGAAAGTTAAGTATGTCACTGAGGGCATGCGTAAACCCG
CCTTTCTAAGCGGAGAACAGAAGAAAGCAATAGTAGATCTGTTATTCAAGACCAACCGCAAA
GTGACAGTTAAGCAATTGAAAGAGGACTACTTTAAGAAAATTGAATGCTTCGATTCTGTCGA
GATCTCCGGGGTAGAAGATCGATTTAATGCGTCACTTGGTACGTATCATGACCTCCTAAAGA
TAATTAAAGATAAGGACTTCCTGGATAACGAAGAGAATGAAGATATCTTAGAAGATATAGTG
TTGACTCTTACCCTCTTTGAAGATCGGGAAATGATTGAGGAAAGACTAAAAACATACGCTCA
CCTGTTCGACGATAAGGTTATGAAACAGTTAAAGAGGCGTCGCTATACGGGCTGGGGACGAT
TGTCGCGGAAACTTATCAACGGGATAAGAGACAAGCAAAGTGGTAAAACTATTCTCGATTTT
CTAAAGAGCGACGGCTTCGCCAATAGGAACTTTATGCAGCTGATCCATGATGACTCTTTAAC
CTTCAAAGAGGATATACAAAAGGCACAGGTTTCCGGACAAGGGGACTCATTGCACGAACATA
TTGCGAATCTTGCTGGTTCGCCAGCCATCAAAAAGGGCATACTCCAGACAGTCAAAGTAGTG
GATGAGCTAGTTAAGGTCATGGGACGTCACAAACCGGAAAACATTGTAATCGAGATGGCACG
CGAAAATCAAACGACTCAGAAGGGGCAAAAAAACAGTCGAGAGCGGATGAAGAGAATAGAAG
AGGGTATTAAAGAACTGGGCAGCCAGATCTTAAAGGAGCATCCTGTGGAAAATACCCAATTG
CAGAACGAGAAACTTTACCTCTATTACCTACAAAATGGAAGGGACATGTATGTTGATCAGGA
ACTGGACATAAACCGTTTATCTGATTACGACGTCGATCACATTGTACCCCAATCCTTTTTGA
AGGACGATTCAATCGACAATAAAGTGCTTACACGCTCGGATAAGAACCGAGGGAAAAGTGAC
AATGTTCCAAGCGAGGAAGTCGTAAAGAAAATGAAGAACTATTGGCGGCAGCTCCTAAATGC
GAAACTGATAACGCAAAGAAAGTTCGATAACTTAACTAAAGCTGAGAGGGGTGGCTTGTCTG
AACTTGACAAGGCCGGATTTATTAAACGTCAGCTCGTGGAAACCCGCCAAATCACAAAGCAT
GTTGCACAGATACTAGATTCCCGAATGAATACGAAATACGACGAGAACGATAAGCTGATTCG
GGAAGTCAAAGTAATCACTTTAAAGTCAAAATTGGTGTCGGACTTCAGAAAGGATTTTCAAT
TCTATAAAGTTAGGGAGATAAATAACTACCACCATGCGCACGACGCTTATCTTAATGCCGTC
GTAGGGACCGCACTCATTAAGAAATACCCGAAGCTAGAAAGTGAGTTTGTGTATGGTGATTA
CAAAGTTTATGACGTCCGTAAGATGATCGCGAAAAGCGAACAGGAGATAGGCAAGGCTACAG
CCAAATACTTCTTTTATTCTAACATTATGAATTTCTTTAAGACGGAAATCACTCTGGCAAAC
GGAGAGATACGCAAACGACCTTTAATTGAAACCAATGGGGAGACAGGTGAAATCGTATGGGA
TAAGGGCCGGGACTTCGCGACGGTGAGAAAAGTTTTGTCCATGCCCCAAGTCAACATAGTAA
AGAAAACTGAGGTGCAGACCGGAGGGTTTTCAAAGGAATCGATTCTTCCAAAAAGGAATAGT
GATAAGCTCATCGCTCGTAAAAAGGACTGGGACCCGAAAAAGTACGGTGGCTTCGATAGCCC
TACAGTTGCCTATTCTGTCCTAGTAGTGGCAAAAGTTGAGAAGGGAAAATCCAAGAAACTGA
AGTCAGTCAAAGAATTATTGGGGATAACGATTATGGAGCGCTCGTCTTTTGAAAAGAACCCC
ATCGACTTCCTTGAGGCGAAAGGTTACAAGGAAGTAAAAAAGGATCTCATAATTAAACTACC
AAAGTATAGTCTGTTTGAGTTAGAAAATGGCCGAAAACGGATGTTGGCTAGCGCCGGAGAGC
TTCAAAAGGGGAACGAACTCGCACTACCGTCTAAATACGTGAATTTCCTGTATTTAGCGTCC
CATTACGAGAAGTTGAAAGGTTCACCTGAAGATAACGAACAGAAGCAACTTTTTGTTGAGCA
GCACAAACATTATCTCGACGAAATCATAGAGCAAATTTCGGAATTCAGTAAGAGAGTCATCC
TAGCTGATGCCAATCTGGACAAAGTATTAAGCGCATACAACAAGCACAGGGATAAACCCATA
CGTGAGCAGGCGGAAAATATTATCCATTTGTTTACTCTTACCAACCTCGGCGCTCCAGCCGC
ATTCAAGTATTTTGACACAACGATAGATCGCAAACGATACACTTCTACCAAGGAGGTGCTAG
ACGCGACACTGATTCACCAATCCATCACGGGATTATATGAAACTCGGATAGATTTGTCACAG
CTTGGGGGTGACGGATCCCCCAAGAAGAAGAGGAAAGTCTCGAGCGACTACAAAGACCATGA
CGGTGATTATAAAGATCATGACATCGATTACAAGGATGACGATGACAAGGCTGCAGGA
(SEQ ID NO: 4)
MDKKYSIGLAIGTNSVGWAVITDEYKVPSKKEKVLGNTDRHSIKKNLIGALLEDSGETAEAT
RLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKERGHFLIEGDLNPDNSDVDKLFI
QLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNRK
VTVKQLKEDYFKKIECFDSVEISGVEDRENASLGTYHDLLKIIKDKDELDNEENEDILEDIV
LTLTLFEDREMIEERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDERKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDKPI
REQAENIIHLFTLTNLGAPAAFKYEDTTIDRKRYTSTKEVLDATLIHQSITGLYETRIDLSQ
LGGD (single underline: HNH domain; double underline: RuvC domain)

In some embodiments, dCas9 corresponds to, or comprises in part or in whole, a Cas9 amino acid sequence having one or more mutations that inactivate the Cas9 nuclease activity. For example, in some embodiments, a dCas9 domain comprises D10A and/or H820A mutation. dCas9 (D10A and H840A):

(SEQ ID NO: 5)
MDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKERGHFLIEGDLNPDNSDVDKLFI
QLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEF
YKFIKPILEKMDGTEELLVKLNREDLLRKQRTEDNGSIPHQIHLGELHAILRRQEDFYPELK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDFLDNEENEDILEDIV
LTLTLFEDREMIEERLKTYAHLEDDKVMKQLKRRRYTGWGRLSRKLINGIRDKQSGKTILDE
LKSDGFANRNEMQLIHDDSLTEKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENTQL
QNEKLYLYYLQNGRDMYVDQELDINRLSDYDVDAIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGESKESILPKRNS
DKLIARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDKPI
REQAENIIHLFTLTNLGAPAAFKYEDTTIDRKRYTSTKEVLDATLIHQ
SITGLYETRIDLSQLGGD (single underline: HNH domain; double underline: RuvC
domain)

In other embodiments, dCas9 variants having mutations other than D10A and H820A are provided, which e.g., result in nuclease inactivated Cas9 (dCas9). Such mutations, by way of example, include other amino acid substitutions at D10 and H820, or other substitutions within the nuclease domains of Cas9 (e.g., substitutions in the HNH nuclease subdomain and/or the RuvC1 subdomain). In some embodiments, variants or homologues of dCas9 (e.g., variants of SEQ ID NO: 5) are provided which are at least about 70% identical, at least about 80% identical, at least about 90% identical, at least about 95% identical, at least about 98% identical, at least about 99% identical, at least about 99.5% identical, or at least about 99.9% to SEQ ID NO:5. In some embodiments, variants of dCas9 (e.g., variants of SEQ ID NO: 5) are provided having amino acid sequences which are shorter, or longer than SEQ ID NO: 5, by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

In some embodiments. Cas9 fusion proteins as provided herein comprise the full-length amino acid of a Cas9 protein, e.g., one of the sequences provided above. In other embodiments, however, fusion proteins as provided herein do not comprise a full-length Cas9 sequence, but only a fragment thereof. For example, in some embodiments, a Cas9 fusion protein provided herein comprises a Cas9 fragment, wherein the fragment binds crRNA and tracrRNA or sgRNA, but does not comprise a functional nuclease domain, e.g., in that it comprises only a truncated version of a nuclease domain or no nuclease domain at all. Exemplary amino acid sequences of suitable Cas9 domains and Cas9 fragments are provided herein, and additional suitable sequences of Cas9 domains and fragments will be apparent to those of skill in the art. In some of these embodiments, the fusion protein comprises a transcriptional activator (e.g., VP64), a transcriptional repressor (e.g., KRAB, SID), a nuclease domain (e.g., FokI), base editors, prime editors, a recombinase domain (e.g., Hin, Gin, or Tn3), a deaminase (e.g., a cytidine deaminase or an adenosine deaminase) or an epigenetic modifier domain (e.g., TET1, p300).

In some embodiments, Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref NC_021284.1); Prevotella intermedia (NCBI Ref: NC_017861.1); Spiroplasma taiwanense (NCBI Ref NC_021846.1); Streptococcus iniae (NCBI Ref: NC_021314.1); Belliella baltica (NCBI Ref: NC_018010.1); Psychroflexus torquisl (NCBI Ref: NC_018721.1): Streptococcus thermophilus (NCBI Ref: YP_820832.1); Listeria innocua (NCBI Ref: NP 472073.1); Campylobacter jejuni (NCBI Ref: YP_002344900.1); or Neisseria meningitidis (NCBI Ref: YP_002342100.1).

The term “deaminase” refers to an enzyme that catalyzes a deamination reaction. In some embodiments, the deaminase is a cytidine deaminase, catalyzing the hydrolytic deamination of cytidine or deoxycytidine to uracil or deoxyuracil, respectively.

The terms “RNA-programmable nuclease” and “RNA-guided nuclease” are used interchangeably herein and refer to a nuclease that forms a complex with (e.g., binds or associates with) one or more RNA molecules that are not a target for cleavage. In some embodiments, an RNA-programmable nuclease, when in a complex with an RNA, may be referred to as a nuclease:RNA complex. RNA-programmable nucleases include Cas9 nucleases. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as two or more molecules. Typically, gRNAs that exist as single RNA species comprise two domains: (1) a domain that shares homology to a target nucleic acid (e.g., directs binding of a Cas9 complex to the target); and (2) a domain that binds a Cas9 protein. The gRNA comprises a nucleotide sequence that complements a target site, which mediates binding of the nuclease/RNA complex to said target site and provides the sequence specificity of the nuclease:RNA complex.

The term “recombinase,” as used herein, refers to a site-specific enzyme that mediates the recombination of DNA between recombinase recognition sequences, which results in the excision, integration, inversion, or exchange (e.g., translocation) of DNA fragments between the recombinase recognition sequences. Recombinases can be classified into two distinct families: serine recombinases (e.g., resolvases and invertases) and tyrosine recombinases (e.g., integrases). Examples of serine recombinases include, without limitation, Hin, Gin, Tn3, β-six, CinH, ParA, γδ, Bxb1, φC31, TP901, TG1, φBT1, R4, φRV1, φFC1, MR11, A118, U153, and gp29. Examples of tyrosine recombinases include, without limitation, Cre, FLP, R, Lambda, HK101, HK022, and pSAM2. The serine and tyrosine recombinase names stem from the conserved nucleophilic amino acid residue that the recombinase uses to attack the DNA and which becomes covalently linked to the DNA during strand exchange. Recombinases have numerous applications, including the creation of gene knockouts/knock-ins and gene therapy applications. (See, e.g., Brown et al., “Serine recombinases as tools for genome engineering,” Methods, 53(4):372-379 (2011); Hirano et al., “Site-specific recombinases as tools for heterologous gene integration,” Appl. Microbiol. Biotechnol., 92(2):227-239 (2011); Chavez and Calos, “Therapeutic applications of the OC31 integrase system,” Curr. Gene Ther., 11(5):375-381 (2011); Turan and Bode, “Site-specific recombinases: from tag-and-target-to tag-and-exchange-based genomic modifications,” FASEB J., 25(12):4088-4107 (2011); Venken and Bellen, “Genome-wide manipulations of Drosophila melanogaster with transposons, Flp recombinase, and ΦC31 integrase,” Methods Mol. Biol., 859:203-228 (2012); Murphy, “Phage recombinases and their applications,” Adv. Virus Res., 83:367-414 (2012); Zhang et al., “Conditional gene manipulation: Cre-ating a new biological era,” J. Zhejiang Univ. Sci. B., 13(7):511-524 (2012); Karpenshif and Bernstein, “From yeast to mammals: recent advances in genetic control of homologous recombination.” DNA Repair (Amst), 1; 11(10):781-788 (2012); the entire contents of each are hereby incorporated by reference in their entirety). The recombinases provided herein are not meant to be exclusive examples of recombinases that can be used in embodiments of the invention. The methods and compositions of the invention can be expanded by mining databases for new orthogonal recombinases or designing synthetic recombinases with defined DNA specificities. (See, e.g., Groth et al., “Phage integrases: biology and applications,” J. Mol. Biol., 335, 667-678 (2004); Gordley et al., “Synthesis of programmable integrases,” Proc. Natl. Acad. Sci. USA., 106, 5053-5058 (2009); the entire contents of each are hereby incorporated by reference in their entirety). Other examples of recombinases that are useful in the methods and compositions described herein are known to those of skill in the art, and any new recombinase that is discovered or generated is expected to be able to be used in the different embodiments of the invention. In some embodiments, a recombinase (or catalytic domain thereof) is fused to a Cas9 protein (e.g., dCas9).

The terms “recombine” and “recombination.” in the context of a nucleic acid modification (e.g., a genomic modification), are used to refer to the process by which two or more nucleic acid molecules, or two or more regions of a single nucleic acid molecule, are modified by the action of a recombinase protein. Recombination can result in, inter alia, the insertion, inversion, excision, or translocation of a nucleic acid sequence, e.g., in or between one or more nucleic acid molecules.

As used herein, the terms “approximately,” or “about,” as applied to one or more values of interest, refers to a value that is similar to a stated reference value. In certain embodiments, the term “approximately” or “about” refers to a range of values that fall within 10%, 9%, 8%, 7%. 6%, 5%, 4%. 3%, 2%, 1%, or less in either direction (greater than or less than) of the stated reference value unless otherwise stated or otherwise evident from the context (for example, when such number would exceed 100% of a possible value).

### General Description of the Invention

ARMMs related compositions and methods for producing and using said particles are known in the art, and are described in, but not limited to, U.S. Pat. Nos. 9,737,480; 9,816,080; 10,260,055; PCT Application Publication WO2018/067546; and U.S. Pat. Pub. Nos. 20220119785; 20220170013; 20220220462; and 20220282275; the entire contents of each of which are hereby incorporated by reference in their entirety.

In one aspect of the instant invention, compositions and methods are provided for ARMMs-mediated delivery of therapeutic agents and payloads to cells of the eye, and in particular, to the retina. It is contemplated that the ARMMs based compositions and methods of the present invention provide certain advantages as compared to the administration of viral vectors including, but not limited to, transient expression, circumventing immune privilege, repeat disability, among other advantages. ARMMs comprising an ARRDC1-Cre fusion protein have been functionally administered into the eyes (e.g., subretinal space) of Ai14 Cre reporter mice. Briefly, in the Ai14 system, the expression of the fluorescent protein tdTomato, is activated only by exogenously derived functional Cre recombinase protein. In Ai14 animals, Cre-dependent TdTomato expression in target cells demonstrates uptake of A1-cre loaded ARMM particles by those cells. Preferred embodiments of the invention thusly provide delivery of nuclear cargos capable of editing the nuclear genome of target cells.

### Representative Genetic Diseases of the Eye

In preferred embodiments, the compositions and methods of the present invention are directed to treating, mitigating, ameliorating, or curing a disease or condition caused by one or more abnormalities in a patient's genome. A non-limiting example of genetic targets for ARMM delivered therapeutics include, but are not limited to: CFH (i.e. age-related macular degeneration), ABCA4 (i.e., Stargardt disease, cone-rod dystrophy, age-related macular degeneration, and retinitis pigmentosa); ELOVL4 (i.e., Stargardt disease); USH2A (i.e., autosomal recessive retinitis pigmentosa, and Usher Syndrome); RPGR and RP2 (i.e., x-linked retinitis pigmentosa); GUCY2D, A1PL1, RDH12, RPGR1P1, and CEP290 (i.e., Leber Congenital Amaurosis (“LCA”)); and TULP1, LRAT, IMPDH1 (i.e., potential LCA-like phenotypes). In additional embodiments, the GUCY2D (i.e., autosomal dominant cone-rod dystrophy), RPE63 (i.e., retinitis pigmentosa), and A1PL1 (i.e., cone-rod dystrophy and retinitis pigmentosa) genes are also thought to be associated with additional conditions.

In still further embodiments, compositions and methods of the present invention are contemplated for treating or ameliorating one or more conditions associated with genes, including, but not limited to: BEST1 (i.e., vitelliform macular dystrophy, age-related macular degeneration, autosomal dominant vitreoretinochoroidopathy, and retinitis pigmentosa, nanophthalmos, autosomal recessive, and bestrophinopathy (ARB)); CLRN1 (i.e., Usher syndrome type IIIA (USH3A), and retinitis pigmentosa); CRB1 (i.e., Leber Congenital Amaurosis, cone-rod dystrophy, retinitis pigmentosa); CRX (i.e., Leber Congenital Amaurosis, cone-rod dystrophy, retinitis pigmentosa); PDE6B (i.e., autosomal dominant congenital stationary night blindness, retinitis pigmentosa); PRPH2 (i.e., vitelliform macular dystrophy, cone-rod dystrophy, retinitis pigmentosa); RHO (i.e., autosomal dominant congenital stationary night blindness, autosomal dominant retinitis pigmentosa); RPE65 (i.e., Leber Congenital Amaurosis, fundus albipunctatus, retinitis pigmentosa); and WDR19 (i.e., cranioectodermal dysplasia, asphyxiating thoracic dystrophy, nephronophthisis, retinitis pigmentosa, Senior-Loken syndrome).

Additional embodiments are directed to delivering ARMMs associated payloads and therapeutic agents to retinal pigment epithelium cells and tissues to treat diseases and conditions including, but not limited to, age-related macular degeneration, Best Vitelliform Macular Dystrophy, Sorsby's Fundus Dystrophy, choroidal neovascularization, diabetic macular edema, MERTK retinitis pigmentosa. RPE65 Leber's Congenital Amaurosis, and Bestrophinopathies (e.g., autosomal dominant vitreoretinochoroidopathy). In other embodiments, ARMMs are directed to delivering payloads and therapeutic agents to photoreceptor cells to treat diseases and conditions including, but not limited to, retinitis pigmentosa (e.g., autosomal dominant, X-linked, and/or recessive type), Stargardt Macular Dystrophy. Achromatopsia, Usher Syndrome 1c, and Cone-Rod Dystrophy. In still further embodiments, to delivering ARMMs associated payloads and therapeutic agents to retinal pigment epithelium cells and photoreceptors to treat diseases and conditions including, but not limited to, Usher Syndrome 1b, Choroideremia, Bardet-Biedl Syndrome, and retinitis pigmentosa.

Thus, in some cases, the compositions described here are administered to a subject having or suspected to have a disease of the eve (e.g., as described herein).

In some cases, the compositions described herein are administered to a subject having or suspected to have Stargardt disease. In some cases, the Stargardt disease is Stargardt disease-1, Stargardt disease-3, Stargardt disease-4, or fundulus flavimaculatus (FFM). In some cases, the subject having or suspected to have Stargardt disease has one or more genetic variations in the ABCA4 gene, ELOVL4 gene, BEST1 gene, and/or PRPH2 genes.

In some cases, the compositions described herein are administered to a subject having or suspected to have Usher Syndrome. In some cases, the Usher Syndrome is Usher Syndrome Type 1, Usher Syndrome Type 2, or Usher Syndrome Type 3. In some cases, the subject having or suspected to have Usher Syndrome has one or more genetic variations in the MYO7A, USH1C, CDH23, PCDHJ5, USH2A, ADGRV1, WHRN, and/or CLRN1 genes.

In some cases, the compositions described herein are administered to a patient having one or more of the following genetic variants relative to human Retinal-specific phospholipid-transporting ATPase ABCA4 (UniProt ID P78363), encoded by human ABCA4:

| POS | CHANGE |
|---|---|
| 13-15 | missing |
| 14 | N > K |
| 18 | R > P |
| 18 | R > W |
| 21-2273 | missing |
| 24 | R > H |
| 54 | C > Y |
| 58 | N > K |
| 60 | A > E |
| 60 | A > T |
| 60 | A > V |
| 65 | G > E |
| 68 | P > L |
| 68 | P > R |
| 72 | G > R |
| 72 | G > V |
| 75 | C > G |
| 77 | V > E |
| 89-2273 | missing |
| 96 | N > D |
| 96 | N > H |
| 96 | N > K |
| 97 | Y > C |
| 100 | S > P |
| 108 | D > V |
| 143 | P > L |
| 152 | R > Q |
| 156 | I > V |
| 172 | G > S |
| 184 | S > F |
| 184 | S > R |
| 185-2273 | missing |
| 190 | Q > H |
| 192 | A > T |
| 206 | S > R |
| 212 | R > C |
| 212 | R > H |
| 220 | R > C |
| 223 | K > Q |
| 230 | C > S |
| 240 | I > R |
| 241 | E > D |
| 244 | L > P |
| 245-2273 | missing |
| 246 | A > T |
| 247 | N > S |
| 249 | D > G |
| 290 | R > W |
| 291 | P > L |
| 300 | T > N |
| 309 | P > R |
| 326-2273 | missing |
| 328 | E > V |
| 333 | R > W |
| 336 | S > C |
| 340 | Y > D |
| 345 | Y > S |
| 380 | N > K |
| 407 | A > V |
| 410 | I > T |
| 415 | N > K |
| 418 | F > S |
| 424 | V > A |
| 431-2273 | missing |
| 445 | S > R |
| 471 | E > K |
| 498 | D > E |
| 508 | R > C |
| 511 | R > C |
| 519 | C > R |
| 523 | D > E |
| 525 | F > C |
| 533-2273 | missing |
| 537 | R > C |
| 541 | L > P |
| 548 | W > R |
| 549 | A > P |
| 550 | G > R |
| 572 | R > P |
| 572 | R > Q |
| 572-2273 | missing |
| 593 | P > L |
| 602 | R > Q |
| 602 | R > W |
| 603 | Y > C |
| 607 | G > R |
| 607 | G > W |
| 608 | F > I |
| 616 | E > K |
| 635 | Q > K |
| 636 | Q > H |
| 639-2273 | missing |
| 640 | P > L |
| 641 | C > S |
| 643 | V > M |
| 645 | D > N |
| 653 | R > C |
| 653 | R > H |
| 686 | L > S |
| 690 | G > V |
| 700-2273 | missing |
| 716 | T > M |
| 754 | F > S |
| 764 | C > Y |
| 765 | S > N |
| 2221 | L > P |
| 2229 | L > P |
| POS | CHANGE |
| 765 | S > R |
| 767 | V > D |
| 779-2273 | missing |
| 782-2273 | missing |
| 797 | L > P |
| 808-2273 | missing |
| 816 | G > V |
| 818 | G > E |
| 821 | W > R |
| 824 | I > T |
| 840 | M > R |
| 849 | V > A |
| 851 | G > D |
| 854 | A > T |
| 863 | G > A |
| 863 | missing |
| 873 | F > L |
| 876-2273 | missing |
| 897 | T > I |
| 931 | V > M |
| 935 | V > A |
| 943 | R > Q |
| 943 | R > W |
| 954 | Y > D |
| 957 | Q > R |
| 959 | T > I |
| 965 | N > S |
| 965 | N > Y |
| 970 | T > P |
| 971 | T > N |
| 972 | T > N |
| 973 | L > S |
| 974 | S > P |
| 977 | T > P |
| 978 | G > C |
| 978 | G > D |
| 989 | V > A |
| 991 | G > R |
| 1014 | L > R |
| 1019 | T > A |
| 1019 | T > M |
| 1022 | E > G |
| 1022 | E > K |
| 1029-2273 | missing |
| 1031 | K > E |
| 1036 | E > K |
| 1038 | A > V |
| 1050 | G > D |
| 1055 | R > W |
| 1063 | S > P |
| 1071 | S > L |
| 1072 | V > A |
| 1074 | I > L |
| 1078 | G > E |
| 1087 | E > D |
| 1087 | E > K |
| 1091 | G > E |
| 1094 | P > T |
| 1097 | R > C |
| 1097 | R > S |
| 1098 | R > C |
| 1099 | S > P |
| 1099-2273 | missing |
| 1108 | R > C |
| 1108 | R > H |
| 1108 | R > L |
| 1112 | T > N |
| 1122 | E > K |
| 1129 | R > C |
| 1129 | R > L |
| 1130 | I > T |
| 1140 | C > W |
| 1159 | L > S |
| 1161 | R > H |
| 1177-2273 | missing |
| 1201 | L > R |
| 1203 | G > D |
| 1203 | G > R |
| 1204 | D > N |
| 1250 | L > P |
| 1300 | R > Q |
| 1300-2273 | missing |
| 1332-2273 | missing |
| 1371 | K > N |
| 1380 | P > L |
| 1388 | L > P |
| 1399 | E > K |
| 1406 | H > Y |
| 1408 | W > L |
| 1408 | W > R |
| 1408-2273 | missing |
| 1416 | missing |
| 1429 | V > A |
| 1430 | L > P |
| 1433 | V > I |
| 1439 | G > D |
| 1440 | F > S |
| 1440 | F > V |
| 1442 | N > K |
| 1443 | R > H |
| 1453-2273 | missing |
| 1461-2273 | missing |
| 1479-2273 | missing |
| 1484 | P > S |
| 1486 | P > L |
| 2237 | T > P |
| 2241 | L > V |
| POS | CHANGE |
| 1488 | C > F |
| 1488 | C > R |
| 1488 | C > Y |
| 1490 | C > Y |
| 1503 | P > L |
| 1511 | P > H |
| 1512 | P > R |
| 1513 | Q > R |
| 1525 | L > P |
| 1526 | T > M |
| 1532 | D > N |
| 1537 | T > M |
| 1551 | missing |
| 1556 | R > T |
| 1562 | I > T |
| 1591 | G > R |
| 1598 | A > D |
| 1631 | L > P |
| 1640 | R > Q |
| 1640 | R > W |
| 1652 | Y > D |
| 1652-2273 | missing |
| 1681-1685 | missing |
| 1689 | S > P |
| 1693 | V > I |
| 1696 | S > N |
| 1703 | Q > E |
| 1703 | Q > K |
| 1705 | R > L |
| 1705 | R > Q |
| 1724-2273 | missing |
| 1729 | L > P |
| 1733 | M > T |
| 1736 | S > P |
| 1748 | G > R |
| 1754 | Y > D |
| 1761-1763 | missing |
| 1762 | A > D |
| 1763 | L > P |
| 1773 | A > V |
| 1775 | I > N |
| 1776 | P > L |
| 1779 | Y > H |
| 1779-2273 | missing |
| 1780 | P > A |
| 1794 | A > D |
| 1794 | A > P |
| 1799 | N > D |
| 1805 | N > D |
| 1820 | R > P |
| 1838 | H > D |
| 1838 | H > N |
| 1838 | H > Y |
| 1843 | R > W |
| 1846 | I > T |
| 1868 | N > I |
| 1884 | V > E |
| 1885 | E > K |
| 1886 | G > E |
| 1890 | missing |
| 1896 | V > D |
| 1898 | R > H |
| 1921 | V > G |
| 1921 | V > M |
| 1940 | L > P |
| 1942 | E > Q |
| 1961 | G > E |
| 1961 | G > R |
| 1970 | L > F |
| 1975 | G > R |
| 1977 | G > S |
| 2017 | C > Y |
| 2023 | I > T |
| 2027 | L > F |
| 2030 | R > Q |
| 2030-2273 | missing |
| 2032 | H > R |
| 2033 | L > R |
| 2035 | L > P |
| 2038 | R > W |
| 2040 | R > Q |
| 2040-2273 | missing |
| 2042 | V > G |
| 2050 | V > L |
| 2064 | A > T |
| 2071 | Y > F |
| 2074 | G > V |
| 2077 | R > G |
| 2077 | R > W |
| 2078 | K > E |
| 2096 | E > K |
| 2097 | P > S |
| 2106 | R > C |
| 2107 | R > C |
| 2107 | R > H |
| 2128 | H > R |
| 2131 | E > K |
| 2139 | R > W |
| 2140 | L > Q |
| 2149 | R > L |
| 2150 | C > R |
| 2150 | C > Y |
| 2160 | K > R |
| 2177 | D > N |
| 2188 | F > S |
| 2263 | R > L |
| POS | CHANGE |
| 11 | L > P |
| 339 | W > G |
| 541 | L > P |
| 863 | G > A |
| 943 | R > W |
| 991 | G > R |
| 1038 | A > V |
| 1091 | G > E |
| 1108 | R > C |
| 1253 | T > M |
| 1488 | C > R |
| 1508 | G > C |
| 1562 | I > T |
| 1640 | R > Q |
| 1652-2273 | missing |
| 1940 | L > P |
| 1961 | G > E |
| 1970 | L > F |
| 1971 | L > R |
| 2027 | L > F |
| 2030 | R > Q |
| 2106 | R > C |
| 11 | L > P |
| 339 | W > G |
| 541 | L > P |
| 863 | G > A |
| 943 | R > W |
| 991 | G > R |
| 1038 | A > V |
| 1091 | G > E |
| 1108 | R > C |
| 1253 | T > M |
| 1488 | C > R |
| 1508 | G > C |
| 1562 | I > T |
| 1640 | R > Q |
| 1652-2273 | missing |
| 1940 | L > P |
| 1961 | G > E |
| 1970 | L > F |
| 1971 | L > R |
| 2027 | L > F |
| 2030 | R > Q |
| 2106 | R > C |

| POS | CHANGE |
|---|---|
| 471 | E > K |
| 762 | A > E |
| 818 | G > E |
| 1129 | R > L |
| 1517 | R > S |
| 1562 | I > T |
| 1578 | G > R |
| 1724 | W > C |
| 1898 | R > H |
| 1970 | L > F |
| 1977 | G > S |
| 2047 | I > N |
| 2137 | C > Y |
| 2177 | D > N |

| POS | CHANGE |
|---|---|
| 53-2273 | missing |
| 55 | H > R |
| 63 | S > P |
| 65 | G > E |
| 107-2273 | missing |
| 212 | R > C |
| 218-2273 | missing |
| 320 | S > C |
| 339-2273 | missing |
| 407 | A > V |
| 440 | Y > C |
| 541 | L > P |
| 605-2273 | missing |
| 636 | Q > K |
| 643 | V > G |
| 661 | L > R |
| 863 | G > A |
| 863 | missing |
| 1038 | A > V |
| 1122 | E > K |
| 1145 | L > H |
| 1183 | G > C |
| 1203 | G > E |
| 1368 | R > C |
| 1479-2273 | missing |
| 1490 | C > Y |
| 1562 | I > T |
| 1598 | A > D |
| 1640 | R > Q |
| 1640 | R > W |
| 1650-2273 | missing |
| 1882 | M > I |
| 1961 | G > E |
| 2030-2273 | missing |
| 2043 | P > S |
| 2050 | V > L |
| 2060 | L > R |
| 2107 | R > H |
| 2146 | G > D |
| 2150 | C > Y |
| 2177 | D > N |

| POS | CHANGE |
|---|---|
| 424 | V > A |
| 455 | L > M |
| 552 | V > I |

In some cases, the compositions described herein are administered to a patient having one or more of the following genetic variants relative to human Unconventional myosin-VIIa (UniProt ID Q13402), encoded by human MYO7A:

| POS | CHANGE |
|---|---|
| 25 | G > R |
| 26 | A > E |
| 67 | V > M |
| 90 | R > P |
| 133 | H > D |
| 134 | I > N |
| 163 | G > R |
| 164 | K > R |
| 165 | T > M |
| 198 | A > T |
| 204 | T > A |
| 212 | R > C |
| 212 | R > H |
| 214 | G > R |
| 218-219 | missing |
| 241 | R > C |
| 241 | R > S |
| 269 | missing |
| 397 | A > D |
| 450 | E > Q |
| 457 | A > V |
| 468 | H > HQ |
| 503 | P > L |
| 519 | G > D |
| 651 | L > P |
| 756 | R > W |
| 826 | A > T |
| 946 | M > R |
| 955 | G > S |
| 968 | E > D |
| 1087 | L > P |
| 1170 | E > K |
| 1240 | R > Q |
| 1248 | E > K |
| 1288 | A > P |
| 1327 | E > K |
| 1343 | R > S |
| 1346 | missing |
| 1347-1351 | missing |
| 1602 | R > Q |
| 1628 | A > S |
| 1743 | R > W |
| 1812 | E > K |
| 1858 | L > P |
| 1873 | R > W |
| 1883 | R > Q |
| 1887 | P > L |
| 1962 | missing |
| 2137 | G > E |
| 2163 | G > S |
| 2187 | G > D |

In some cases, the compositions described herein are administered to a patient having one or more of the following genetic variants relative to human Cadherin-23 (UniProt ID Q91H251), encoded by human CDH23:

| POS | CHANGE |
|---|---|
| 247 | E > K |
| 366 | A > T |
| 484 | A > P |
| 755 | H > Y |
| 1090 | V > I |
| 1098 | N > S |
| 1206 | P > R |
| 1209 | T > A |
| 1281 | missing |
| 1496 | Q > H |
| 1507 | R > Q |
| 1746 | R > Q |
| 1788 | P > L |
| 1912 | R > W |
| 1930 | D > N |
| 2017 | G > S |
| 2376 | D > V |
| 2517 | S > G |
| 2530 | T > I |
| 2744 | G > S |
| 2771 | G > S |
| 2833 | R > G |
| 2968 | V > A |
| 3175 | R > H |
| 3189 | R > W |
| 3245 | S > F |

In some cases, the compositions described herein are administered to a patient having one or more of the following genetic variants relative to human Protocadherin-15 (UniProt ID Q96QU1), encoded by human PCDH15:

| POS | CHANGE |
|---|---|
| 134 | R > Q |
| 1342 | Q > K |
| 1611 | E > A |
| 1867 | missing |
| 178 | D > G |

In some cases, the compositions described herein are administered to a patient having one or more of the following genetic variants relative to human Usherin (UniProt ID 075445), encoded by human USH2A:

| POS | CHANGE |
|---|---|
| 44 | G > R |
| 163 | C > Y |
| 180 | S > P |
| 218 | V > E |
| 230 | V > M |
| 268 | G > R |
| 280 | L > F |
| 284 | E > K |
| 303 | R > C |
| 303 | R > S |
| 307 | S > I |
| 319 | C > Y |
| 334 | R > Q |
| 334 | R > W |
| 346 | N > H |
| 352 | T > I |
| 357 | N > T |
| 382 | V > M |
| 391 | S > I |
| 419 | C > F |
| 464 | R > C |
| 478 | E > D |
| 516 | G > V |
| 517 | R > T |
| 536 | C > R |
| 555 | L > V |
| 575 | C > S |
| 587 | missing |
| 610 | H > P |
| 691 | C > Y |
| 759 | C > F |
| 761 | P > R |
| 1059 | P > L |
| 1212 | P > L |
| 1369 | missing |
| 1515 | T > M |
| 1734 | G > R |
| 1777 | R > W |
| 1833 | V > E |
| 1836 | P > T |
| 1840 | G > V |
| 1843 | P > L |
| 1861 | G > S |
| 1953 | A > G |
| 2080 | K > N |
| 2116 | H > R |
| 2128 | C > F |
| 2128 | C > Y |
| 2196 | S > T |
| 2238 | E > A |
| 2249 | A > D |
| 2260 | S > P |
| 2265-2266 | EY > D |
| 2292 | R > H |
| 2354 | R > H |
| 2562 | V > A |
| 2639 | S > P |
| 2738 | D > N |
| 2744 | W > C |
| 2752 | G > R |
| 2786 | F > S |
| 2795 | A > S |
| 3124 | R > G |
| 3251 | C > R |
| 3263-3269 | missing |
| 3267 | C > R |
| 3282 | C > R |
| 3448 | E > K |
| 3462 | T > I |
| 3472 | Y > YY |
| 3479 | W > C |
| 3504 | P > T |
| 3515 | D > G |
| 3521 | W > R |
| 3529 | G > S |
| 3546 | G > R |
| 3571 | T > M |
| 3747 | Y > C |
| 3844 | I > M |
| 3894 | N > D |
| 3895 | G > E |
| 3904 | R > K |
| 3976 | T > M |
| 4054 | S > I |
| 4115 | R > C |
| 4174 | S > R |
| 4232 | P > R |
| 4269 | P > R |
| 4337 | T > M |
| 4386 | I > F |
| 4425 | T > M |
| 4433 | V > L |
| 4439 | T > I |
| 4445-4449 | ENMDS > DL |
| 4487 | Y > C |
| 4570 | R > H |
| 4592 | Q > H |
| 4662 | Q > E |
| 4692 | G > R |
| 4763 | G > R |
| 4795 | L > R |
| 4808 | C > R |
| 4817 | G > R |
| 4818 | P > L |
| 4918 | T > M |
| 5143 | R > C |

In some cases, the compositions described herein are administered to a patient having one or more of the following genetic variants relative to human Adhesion G-protein coupled receptor V1 (UniProt ID Q8WXG9), encoded by human ADGRV1:

| POS | CHANGE |
|---|---|
| 4789 | R > W |
| 5978 | H > R |

In some cases, the compositions described herein are administered to a patient having one or more of the following genetic variants relative to human Clarin-1 (UniProt ID P58418), encoded by human CLRN1:

| CHANGE | DESCRIPTION |
|---|---|
| 40 | C > G |
| 48 | N > K |
| 105 | S > P |
| 120 | M > K |
| 150 | L > P |
| 153-154 | IL > M |
| 168 | I > N |

In some cases, the composition administered to the subject comprises a therapeutic payload that targets one or more of the genetic variants described herein. In some cases, the therapeutic payload targets one or more of the genetic variants described herein to modulate its expression (e.g., by overexpression or inhibition). In some cases, the therapeutic payload targets one or more of the genetic variants described herein for genome editing (e.g., of the genetic variant). In some cases, the therapeutic payload is a genome editor or portion thereof (e.g., as described herein, e.g., an RNA guided genome editor or portion thereof, e.g., a nuclease, a base editor, a prime editor, or a portion thereof).

### General Pharmaceutical Formulations and Compositions

Additional aspects of the present disclosure relate to pharmaceutical compositions comprising any of the ARMMs or microvesicle (e.g., ARMM) producing cells provided herein. The term “pharmaceutical composition,” as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, potency, efficacy, biological effect, and the like, as well as other therapeutic agents and compounds).

As used here, the term “pharmaceutically-acceptable carrier” means a pharmaceutically-acceptable material, composition, or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue, system, or portion of the body). A pharmaceutically acceptable carrier is “acceptable” in the sense of being compatible with the other ingredients of the formulation and not injurious to the cell and tissue of the subject (e.g., physiologically compatible, sterile, physiologic pH, etc.). Some examples of materials which can serve as pharmaceutically-acceptable carriers include, but are not limited to: (1) sugars, such as lactose, glucose and sucrose; (2) starches, such as com starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar: (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-CU alcohols, such as ethanol; and (24) other non-toxic compatible substances employed in pharmaceutical formulations. Wetting agents, coloring agents, release agents, coating agents, sweetening agents, flavoring agents, perfuming agents, preservatives, antioxidants, and the like, can also optionally be present in a formulation. Terms such as “excipient,” “carrier,” “pharmaceutically acceptable carrier,” and the like, are used interchangeably herein.

In some embodiments, the pharmaceutical composition is formulated for delivery to a subject e.g., for delivering a therapeutic agent, payload protein, or payload nucleic acid to a cell. Suitable routes of administrating of the pharmaceutical compositions described herein include, without limitation: subretinal, suprachoroidal, intrvitereal, topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. (See, e.g., Hartman, R. R., and Kompella, U. B., “Intravitreal, Subretinal, and Suprachoroidal Injections: Evolution of Microneedles for Drug Delivery,” J. Ocul. Pharmacol. Ther., 34(1-2):141-153 (2018)).

In some embodiments, a pharmaceutical composition described herein is administered locally to a diseased site (e.g., cell of the eye). In some embodiments, the pharmaceutical compositions described herein are administered to a subject by injection, by means of a catheter, by means of a suppository, or by means of an implant, the implant being of a porous, non-porous, or gelatinous material, including a membrane, such as a sialastic membrane, or a fiber.

In some embodiments, the compositions are formulated in accordance with routine procedures and is a composition adapted for injection, intravenous, or subcutaneous administration to a subject, e.g., a human. In some embodiments, the compositions for administration by injection comprise a solution in a sterile isotonic aqueous buffer. Where necessary, the formulations can also include a solubilizing agent and a local anesthetic such as lidocaine to ease pain at the site of the injection. Where the formulations are administered by injection, an ampoule of sterile water for injection or saline can be provided so that the ingredients can be mixed prior to administration. Where the formulations are to be administered by infusion, they can be dispensed with an infusion bottle containing sterile pharmaceutical grade water or saline.

A formulation (a pharmaceutical composition) for systemic administration may be a liquid, e.g., sterile saline, lactated Ringer's solution, or Hank's solution. In addition, the pharmaceutical composition can be in solid forms and re-dissolved or suspended immediately prior to use. Lyophilized forms of certain formulations are also contemplated.

The compositions described herein may be administered or packaged as a unit dose. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent. i.e., carrier, or vehicle.

Further, the compositions can be provided as kits comprising: 1) a container containing an ARMM or microvesicle producing cell of the invention; and 2) a second container containing a pharmaceutically acceptable diluent (e.g. sterile water) for injection. The pharmaceutically acceptable diluent can be used, e.g., for reconstitution or dilution of the ARMM or microvesicle producing cell of the invention. Optionally associated with such container(s) can be a notice in the form prescribed by a governmental agency regulating the manufacture, use, or sale of pharmaceuticals or biological products, which notice reflects approval by the agency of manufacture, use, or sale for human administration. In this regard, state-specific and regional regulatory agencies are understood to include, but are not limited to, the U.S. Food and Drug Administration, the U.S. Department of Agriculture, the European Medicines Agency, the United Kingdom Medicines and Healthcare Products Regulatory Agency, the National Medical Products Administration, and the like.

In another aspect, an article of manufacture containing materials useful for treating the diseases described herein is included. In some embodiments, the article of manufacture comprises a container and a label. Suitable containers include, for example, bottles, vials, syringes, and test tubes. The containers may be formed from various materials such as glass or plastic. Suitable containers are further understood to include materials that are sufficiently non-reactive and protective of the contents therein. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the invention. In some embodiments, the label on or associated with the container indicates that the composition is used for treating one or more diseases of choice. The article of manufacture may further comprise a second container comprising a pharmaceutically acceptable buffer, such as phosphate-buffered saline, Ringer's solution, or dextrose solution. It may further include other materials desirable from a commercial and user standpoint, including other buffers, diluents, filters, needles, syringes, and package inserts with instructions and indications for acceptable, recognized, or permitted use(s).

In some embodiments, treatments are contemplated wherein one therapeutic agent is administered using ARMM-mediated delivery of an agent to target cells, tissues, systems, or mammalian subjects.

In some other embodiments, treatments are contemplated wherein two therapeutic agents are administered using ARMM-mediated delivery of the respective agents to target cells, tissues, systems, or mammalian subjects.

In still some other embodiments, treatments are contemplated wherein three or more therapeutic agents are administered using ARMM-mediated delivery of the respective agents to target cells, tissues, systems, or mammalian subjects.

It is contemplated that within the compositions and methods described herein, when two or more respective therapeutic agents are administered (e.g., co-administered) to a target, cell, tissue, system, or subject that the respective (i.e., two or more) agents are provided in a single ARRDC1-mediated microvesicle (ARMM) particle for administration.

It is likewise further contemplated that within the compositions and methods described herein, when two or more respective therapeutic agents are administered (e.g., concomitantly administered) to a target cell, tissue, system, or subject that the respective (i.e., two or more) agents are provided respectively in different ARRDC1-mediated microvesicles (ARMMs) particles for administration.

### Kits, Vectors, and Cells

Some aspects of this disclosure provide kits comprising a nucleic acid construct comprising a nucleotide sequence encoding one or more of any of the proteins (e.g., ARRDC1, and TSG110), fusion proteins and/or nucleic acids provided herein. In some embodiments, the nucleotide sequence encodes any of the proteins, fusion proteins, and/or RNAs provided herein. In some embodiments, the nucleotide sequence comprises a heterologous promoter that drives the expression of any of the proteins, fusion proteins, and/or RNAs provided herein.

Some aspects of this disclosure provide microvesicle (e.g., ARMM) producing cells comprising any of the proteins, fusion proteins, and nucleic acids (e.g., RNAs) provided herein. In some embodiments, the cells specifically comprise a nucleotide that encodes any of the proteins, fusion proteins, and/or RNAs provided herein. In some embodiments, the cells comprise any of the nucleotides or vectors provided herein. In some embodiments, the vector comprises one or more cell targeting or cell entry proteins (e.g., viral and/or human fusogen(s)).

It should be appreciated, however, that additional proteins, fusion proteins, and RNAs would be apparent to the skilled artisan based on the present disclosure and knowledge in the art.

The function and advantage of these and other embodiments of the present invention will be more fully understood from the Examples below. The following Examples are intended to illustrate the benefits of the present invention and to describe particular embodiments but are not intended to exemplify the full scope of the invention.

Accordingly, it will be understood that the Examples are not meant to limit the scope of the invention.

### DETAILED DESCRIPTION OF CERTAIN EMBODIMENTS OF THE INVENTION

The present invention provides methods, systems, and compositions for ARRDC1-mediated microvesicles (“ARMMs”) delivery of molecules of interest (e.g., therapeutic agents) to cells and tissues of the eye. The present invention further relates to compositions and methods of producing, testing, and administering ARMMs to one or more internal structures of the eve. More particularly, the present invention provides compositions and methods of producing, testing, and administering ARMMs comprising one or more therapeutic agents (e.g., biological molecules including, but not limited to, CRISPR/Cas9, and other similar endonucleases, base editors, small molecules, proteins, and nucleic acids (e.g., DNA, RNA, siRNA, mRNA, miRNA, and the like)). Also provided are methods of administering therapeutic agents associated with ARMMs, including, but not limited to, methods of treating or contacting cells and tissues of the eye in one or more exemplary dosing regimens (e.g., 1) in vivo administration of ARMMs to a patient; 2) ex vivo administration of ARMMs to target cells and implantation of the ARMMs treated cells to a/the patient; and 3) in vivo and ex vivo regimens). In particular, the present invention provides methods of administering therapeutic agents via ARMMs to the cells and tissues that comprise the retina or into the subretinal space. Additionally, the present invention relates to methods of manufacturing (e.g., culturing, clarifying, separating, and concentrating) the inventive compositions obtained from stable producer cell lines and/or from transient cell cultures.

More particularly, the instant disclosure relates to the discovery that therapeutic agents associated with an ARRDC1 protein can be loaded into ARMMs and delivered to cells and tissues comprising the retina. In some embodiments, the uptake of these ARMMs and associated their payload(s) is enhanced by the presence of portions of viral envelope proteins, including, but not limited to, VSV-G on the surface of ARMMs. Different therapeutic agents, such as proteins and nucleic acids, including various RNAs, can be loaded in such ARMMs for delivery to retina cells. In preferred embodiments, various types of endonucleases (e.g., CRISPR/Cas9 with associated gRNA), base and prime editors with associated gRNA, and effector proteins, are contemplated as being suitable for delivery using the compositions and methods of the present invention to the cells and tissues of the eye, and more particularly, to the cells of the retina and subretinal space.

### ARMMs

Arrestin Domain Containing Protein 1 mediated microvesicles (“ARMMs”) are extracellular vesicles (“EVs”) that are distinct from exosomes. The budding of ARMMs requires ARRDC1, which is localized to the cytosolic side of the plasma membrane and, through a tetrapeptide motif, recruits the ESCRT-I complex protein TSG101 to the cell surface to initiate the outward membrane budding. Thus, in contrast to exosomes, the biogenesis of ARMMs occurs at the plasma membrane. ARMMs exhibit several additional features that make them potentially ideal vehicles for therapeutic delivery. ARRDC1 is not only necessary but also sufficient to drive ARMMs budding. Overexpression of the ARRDC1 protein increases the production of ARMMs in cells. This allows controlled production of ARMMs using modem biological manufacturing methods. Moreover, endogenous proteins such as cell surface receptors are actively recruited into ARMMs and can be delivered into recipient cells to initiate intercellular communication, suggesting that the exogenous payload molecules may be similarly packaged and delivered via ARMMs.

### ARRDC1

In preferred embodiments, ARRDC1 is a protein that comprises a PSAP (SEQ ID NO: 1) motif and a PPXY (SEQ ID NO: 2) motif in its C-terminus, and interacts with TSG101 as shown herein. It should be appreciated that the PSAP (SEQ ID NO: 1) motif and the PPXY (SEQ ID NO: 2) motif are not required to be at the absolute C-terminal end of the ARRDC1. Rather, they may be at a C-terminal portion of the ARRDC1 protein (e.g., the C-terminal half of the ARRDC1). The disclosure also contemplates variants of ARRDC1, such as fragments of ARRDC1 and/or ARRDC1 proteins that have a degree of identity (e.g., 60%, 70%, 80%, 85%, 90%, 95%, 98%, or 99% identity) to an ARRDC1 protein and are capable if interacting with TSG101. Accordingly, an ARRDC1 protein may be a protein that comprises a PSAP (SEQ ID NO: 1) motif and a PPXY (SEQ ID NO: 2) motif and interacts with TSG101. In some embodiments, the ARRDC1 protein is at least 60%, 65%, 70%, 75%, 80%, 85%, 90%, 95%, 98%, or 99% identical to the amino acid sequence of any one of SEQ ID NOs: 42-44, comprises a PSAP (SEQ ID NO: 1) motif and a PPXY (SEQ ID NO: 2) motif, and interacts with TSG101. In some embodiments, the ARRDC1 protein has at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, or at least 430 identical contiguous amino acids of any one of SEQ ID NOs: 42-44, comprises a PSAP (SEQ ID NO: 1) motif and a PPXY (SEQ ID NO: 2) motif, and interacts with TSG101. In some embodiments, the ARRDC1 protein has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to any one of the amino acid sequences set forth in SEQ ID NOs: 42-44 comprises a PSAP (SEQ ID NO: 1) motif and a PPXY (SEQ ID NO: 2) motif, and interacts with TSG101. In some embodiments, the ARRDC1 protein comprises any one of the amino acid sequences set forth in SEQ ID NOs: 42-44. Exemplary, non-limiting ARRDC1 protein sequences are provided herein, and additional, suitable ARRDC1 protein variants according to aspects of this invention are known in the art. It will be appreciated by those of skill in the art that this invention is not limited in this respect. Exemplary ARRDC1 sequences include the following (PSAP (SEQ ID NO: 1) and PPXY (SEQ ID NO: 2) motifs are marked):

>gi|22748653|ref NP_689498.1| arrestin domain-containing protein 1 [Homo
sapiens]
(SEQ ID NO: 42)
MGRVOLFEISLSHGRVVYSPGEPLAGTVRVRLGAPLPFRAIRVTCIGSCGVSNKANDTAWVV
EEGYENSSLSLADKGSLPAGEHSFPFQFLLPATAPTSFEGPFGKIVHQVRAAIHTPRESKDH
KCSLVFYILSPLNLNSIPDIEQPNVASATKKESYKLVKTGSVVLTASTDLRGYVVGQALQLH
ADVENQSGKDTSPVVASLLQKVSYKAKRWIHDVRTIAEVEGAGVKAWRRAQWHEQILVPALP

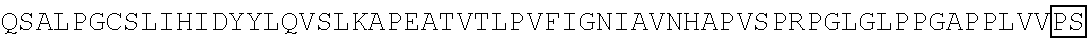

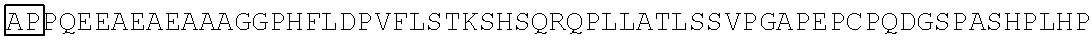

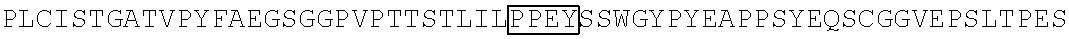

>gi|244798004|ref|NP_001155957.1| arrestin domain-containing protein 1 isoform
a [Mus musculus]
(SEQ ID NO: 43)
MGRVOLFEIRLSQGRVVYGPGEPLAGTVHLRLGAPLPFRAIRVTCMGSCGVSTKANDGAWVV
EESYFNSSLSLADKGSLPAGEHNFPFQFLLPATAPTSFEGPFGKIVHQVRASIDTPRESKDH
KCSLVFYILSPLNLNSIPDIEQPNVASTTKKFSYKLVKTGNVVLTASTDLRGYVVGQVLRLQ
ADIENQSGKDTSPVVASLLQKVSYKAKRWIYDVRTIAEVEGTGVKAWRRAQWQEQILVPALP

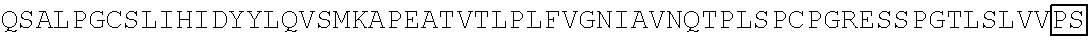

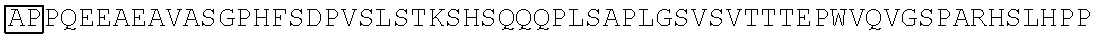

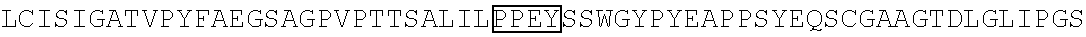

>gi|244798112|ref|NP_848495.2| arrestin domain-containing protein 1 isoform b
[Mus musculus]
(SEQ ID NO: 44)
MGRVOLFEIRLSQGRVVYGPGEPLAGTVHLRLGAPLPFRAIRVTCMGSCGVSTKANDGAWVV
EESYENSSLSLADKGSLPAGEHNFPFQFLLPATAPTSFEGPFGKIVHQVRASIDTPRESKDH
KCSLVFYILSPLNLNSIPDIEQPNVASTTKKFSYKLVKTGNVVLTASTDLRGYVVGQVLRLQ
ADIENQSGKDTSPVVASLLQVSYKAKRWIYDVRTIAEVEGTGVKAWRRAQWQEQILVPALPQ

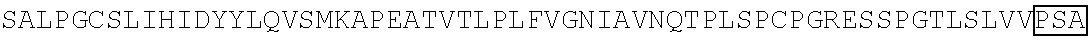

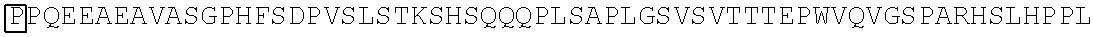

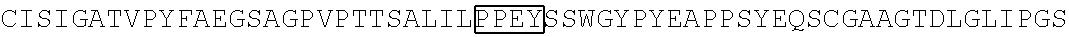

### TSG101

In certain embodiments, the inventive microvesicles further comprise TSG101 (tumor susceptibility gene 101) TSG101 belongs to a group of putative inactive homologs of ubiquitin-conjugating enzymes. The protein contains a coiled-coil domain that interacts with stathmin, a cytosolic phosphoprotein implicated in tumorigenesis. TSG101 is a protein that comprises a UEV domain and interacts with ARRDC1. As referred to herein, UEV refers to the Ubiquitin E2 variant domain of approximately 145 amino acids. The structure of the domain contains a α/β fold similar to the canonical E2 enzyme but has an additional N-terminal helix and further lacks the two C-terminal helices. Often found in TSG101/Vps23 proteins, the UEV interacts with a ubiquitin molecule and is essential for the trafficking of a number of ubiquitylated payloads to multivesicular bodies (MVBs). Furthermore, the UEV domain can bind to Pro-Thr/Ser-Ala-Pro peptide ligands, a fact exploited by viruses such as HIV. Thus, the TSG101 UEV domain binds to the PTAP tetrapeptide motif in the viral Gag protein that is involved in viral budding. The disclosure also contemplates variants of TSG101, such as fragments of TSG101 and/or TSG101 proteins that have a degree of identity (e.g., 60%, 70%, 80%, 85%, 90%, 95%, 98%, or 99% identity) to a TSG101 protein and are capable of interacting with ARRDC1. Accordingly, a TSG101 protein may be a protein that comprises a UEV domain and interacts with ARRDC1. In some embodiments, the TSG101 protein is at least 50%, 55%, 60%, 65%, 70%, 75%, 80%, 85%, 90%, 95%. 98%, or 99% identical to the amino acid sequence of any one of SEQ ID NOs: 45-47, comprises a UEV domain, and interacts with ARRDC1. In some embodiments, the TSG101 protein has at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, or at least 390, and any integer between the numbers, identical contiguous amino acids of any one of SEQ ID NOs: 45-47, comprises a UEV domain and interacts with ARRDC1. In some embodiments, the TSG101 protein has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to any one of the amino acid sequences set forth in SEQ ID NOs: 45-47 and comprises a UEV domain. In some embodiments, the ARRDC1 protein comprises any one of the amino acid sequences set forth in SEQ ID NOs: 45-47. Exemplary, non-limiting TSG101 protein sequences are provided herein, and additional, suitable TSG101 protein sequences, isoforms, and variants are known in the art. It will be appreciated by those of skill in the art that this invention is not limited in this respect. Exemplary TSG101 sequences include the following sequences (the UEV domain in these sequences includes amino acids 1-145 and is underlined in the sequences below):

>gi|5454140|ref|NP_006283.1|
tumor susceptibility gene 101 protein [Homo
sapiens]
(SEQ ID NO: 45)
MAVSESQLKKMVSKYKYRDLTVRETVNVITLYKDLKPVLDSYVENDGSSRELMNLTGTIPVP
YRGNTYNIPICLWLLDTYPYNPPICFVKPTSSMTIKTGKHVDANGKIYLPYLHEWKHPQSDL
LGLIQVMIVVFGDEPPVFSRPISASYPPYQATGPPNTSYMPGMPGGISPYPSGYPPNPSGYP
GCPYPPGGPYPATTSSQYPSQPPVTTVGPSRDGTISEDTIRASLISAVSDKLRWRMKEEMDR
AQAELNALKRTEEDLKKGHQKLEEMVTRLDQEVAEVDKNIELLKKKDEELSSALEKMENQSE
NNDIDEVIIPTAPLYKQILNLYAEENAIEDTIFYLGEALRRGVIDLDVFLKHVRLLSRKQFQ
LRALMQKARKTAGLSDLY
>gi|11230780|ref|NP_068684.1|
tumor susceptibility gene 101 protein [Mus
musculus]
(SEQ ID NO: 46)
MAVSESQLKKMMSKYKYRDLTVRQTVNVIAMYKDLKPVLDSYVENDGSSRELVNLTGTIPVR
YRGNIYNIPICLWLLDTYPYNPPICFVKPTSSMTIKTGKHVDANGKIYLPYLHDWKHPRSEL
LELIQIMIVIFGEEPPVFSRPTVSASYPPYTATGPPNTSYMPGMPSGISAYPSGYPPNPSGY
PGCPYPPAGPYPATTSSQYPSQPPVTTVGPSRDGTISEDTIRASLISAVSDKLRWRMKEEMD
GAQAELNALKRTEEDLKKGHQKLEEMVTRLDQEVAEVDKNIELLKKKDEELSSALEKMENQS
ENNDIDEVIIPTAPLYKQILNLYAEENAIEDTIFYLGEALRRGVIDLDVELKHVRLLSRKQF
QLRALMQKARKTAGLSDLY
>gi|48374087|ref|NP_853659.2|
tumor susceptibility gene 101 protein [Rattus
norvegicus]
(SEQ ID NO: 47)
MAVSESQLKKMMSKYKYRDLTVRQTVNVIAMYKDLKPVLDSYVENDGSSRELVNLTGTIPVR
YRGNIYNIPICLWLLDTYPYNPPICFVKPTSSMTIKTGKHVDANGKIYLPYLHDWKHPRSEL
LELIQIMIVIFGEEPPVFSRPTVSASYPPYTAAGPPNTSYLPSMPSGISAYPSGYPPNPSGY
PGCPYPPAGPYPATTSSQYPSQPPVTTAGPSRDGTISEDTIRASLISAVSDKLRWRMKEEMD
GAQAELNALKRTEEDLKKGHQKLEEMVTRLDQEVAEVDKNIELLKKKDEELSSALEKMENQS
ENNDIDEVIIPTAPLYKQILNLYAEENAIEDTIFYLGEALRRGVIDLDVELKHVRLLSRKQF
QLRALMQKARKTAGLSDLY

The structure of UEV domains is known to those of skill in the art. (See, e.g., Owen Pornillos et al, Structure and functional interactions of the Tsg101 UEV domain, EMBO J., 21(10): 2397-2406 (2002), the entire contents of which are incorporated herein by reference).

### Expression Constructs

Some aspects of this invention provide expression constructs for encoding a gene product or gene products that induce or facilitate the generation of ARMMs in cells harboring such a construct. In some embodiments, the expression constructs described herein encode a fusion protein as described herein, such as ARRDC1 fusion proteins and TSG101 fusion proteins. In some embodiments, the expression constructs encode an ARRDC1 protein, or variant thereof, and/or a TSG101 protein, or variant thereof. In some embodiments, overexpression of either or both gene products in a cell increases the production of ARMMs in the cell, thus turning the cell into a microvesicle producing cell. In some embodiments, such an expression construct comprises at least one restriction or recombination site that allows in-frame cloning of a protein sequence to be fused, either at the C-terminus, or at the N-terminus of the encoded ARRDC1, or variant thereof. As another example, an expression construct comprises at least one restriction or recombination site that allows in-frame cloning of a protein sequence to be fused either at the C-terminus or at the N-terminus of one or more encoded WW domains.

In some embodiments, the expression construct comprises (a) a nucleotide sequence encoding an ARRDC1 protein, or variant thereof, operably linked to a heterologous promoter, and (b) a restriction site or a recombination site positioned adjacent to the ARRDC1-encoding nucleotide sequence allowing for the insertion of a nucleotide sequencing encoding a payload protein, or an RNA binding protein or RNA binding protein variant sequence, in frame with the ARRDC1-encoding nucleotide sequence. In some embodiments, the heterologous promoter may be a constitutive promoter, in some embodiments, the heterologous promoter may be an inducible promoter. Some aspects of this invention provide an expression construct comprising (a) a nucleotide sequence encoding a TSG101 protein, or variant thereof, operably linked to a heterologous promoter, and (b) a restriction site or a recombination site positioned adjacent to the TSG101-encoding nucleotide sequence allowing for the insertion of a nucleotide sequencing encoding a payload protein, or an RNA binding protein, DNA binding protein, or variant sequence thereof, in frame with the TSG101-encoding nucleotide sequence. In some embodiments, the heterologous promoter may be a constitutive promoter, in some embodiments, the heterologous promoter may be an inducible promoter.

Some aspects of this invention provide an expression construct comprising (a) a nucleotide sequence encoding a WW domain, or variant thereof, operably linked to a heterologous promoter, and (b) a restriction site or a recombination site positioned adjacent to the WW domain-encoding nucleotide sequence allowing for the insertion of a payload protein or RNA binding protein, or a protein variant sequence thereof in frame with the WW domain-encoding nucleotide sequence. In some embodiments, the heterologous promoter may be a constitutive promoter, in some embodiments, the heterologous promoter may be an inducible promoter. The expression constructs may encode a payload protein, or an RNA binding protein fused to at least one WW domain. In some embodiments, the expression constructs encode a payload protein or an RNA binding protein, or variant thereof, fused to at least one WW domain, or variant thereof. Any of the expression constructs, described herein, may encode any WW domain or variant thereof. In some embodiments, the heterologous promoter may be a constitutive promoter, in some embodiments, the heterologous promoter may be an inducible promoter.

The expression constructs, described herein, may comprise any nucleic acid sequence capable of encoding a WW domain or variant thereof. For example, a nucleic acid sequence encoding a WW domain or WW domain variant may be from the human ubiquitin ligase WWP1, WWP2, Nedd4-1, Nedd4-2, Smurf1. Smurf2, ITCH, NEDL1, or NEDL2. Exemplary nucleic acid sequences of WW domain containing proteins are listed below. It should be appreciated that any of the nucleic acids encoding WW domains or WW domain variants of the exemplary proteins may be used in the invention, described herein, and are not meant to be limiting.

Human WWP1 nucleic acid sequence
(uniprot.org/uniprot/Q9H0M0).
(SEQ ID NO: 48)
GAATTCGCGGCCGCGTCGACCGCTTCTGTGGCCACGGCAGATGAAACAGAAAGGCTAAAG
AGGGCTGGAGTCAGGGGACTTCTCTTCCACCAGCTTCACGGTGATGATATGGCATCTGCC
AGCTCTAGCCGGGCAGGAGTGGCCCTGCCTTTTGAGAAGTCTCAGCTCACTTTGAAAGTG
GTGTCCGCAAAGCCCAAGGTGCATAATCGTCAACCTCGAATTAACTCCTACGTGGAGGTG
GCGGTGGATGGACTCCCCAGTGAGACCAAGAAGACTGGGAAGCGCATTGGGAGCTCTGAG
CTTCTCTGGAATGAGATCATCATTTTGAATGTCACGGCACAGAGTCATTTAGATTTAAAG
GTCTGGAGCTGCCATACCTTGAGAAATGAACTGCTAGGCACCGCATCTGTCAACCTCTCC
AACGTCTTGAAGAACAATGGGGGCAAAATGGAGAACATGCAGCTGACCCTGAACCTGCAG
ACGGAGAACAAAGGCAGCGTTGTCTCAGGCGGAAAACTGACAATTTTCCTGGACGGGCCA
ACTGTTGATCTGGGAAATGTGCCTAATGGCAGTGCCCTGACAGATGGATCACAGCTGCCT
TCGAGAGACTCCAGTGGAACAGCAGTAGCTCCAGAGAACCGGCACCAGCCCCCCAGCACA
AACTGCTTTGGTGGAAGATCCCGGACGCACAGACATTCGGGTGCTTCAGCCAGAACAACC
CCAGCAACCGGCGAGCAAAGCCCCGGTGCTCGGAGCCGGCACCGCCAGCCCGTCAAGAAC
TCAGGCCACAGTGGCTTGGCCAATGGCACAGTGAATGATGAACCCACAACAGCCACTGAT
CCCGAAGAACCTTCCGTTGTTGGTGTGACGTCCCCACCTGCTGCACCCTTGAGTGTGACC
CCGAATCCCAACACGACTTCTCTCCCTGCCCCAGCCACACCGGCTGAAGGAGAGGAACCC
AGCACTTCGGGTACACAGCAGCTCCCAGCGGCTGCCCAGGCCCCCGACGCTCTGCCTGCT
GGATGGGAACAGCGAGAGCTGCCCAACGGACGTGTCTATTATGTTGACCACAATACCAAG
ACCACCACCTGGGAGCGGCCCCTTCCTCCAGGCTGGGAAAAACGCACAGATCCCCGAGGC
AGGTTTTACTATGTGGATCACAATACTCGGACCACCACCTGGCAGCGTCCGACCGCGGAG
TACGTGCGCAACTATGAGCAGTGGCAGTCGCAGCGGAATCAGCTCCAGGGGGCCATGCAG
CACTTCAGCCAAAGATTCCTATACCAGTTTTGGAGTGCTTCGACTGACCATGATCCCCTG
GGCCCCCTCCCTCCTGGTTGGGAGAAAAGACAGGACAATGGACGGGTGTATTACGTGAAC
CATAACACTCGCACGACCCAGTGGGAGGATCCCCGGACCCAGGGGATGATCCAGGAACCA
GCTTTGCCCCCAGGATGGGAGATGAAATACACCAGCGAGGGGGTGCGATACTTTGTGGAC
CACAATACCCGCACCACCACCTTTAAGGATCCTCGCCCGGGGTTTGAGTCGGGGACGAAG
CAAGGTTCCCCTGGTGCTTATGACCGCAGTTTTCGGTGGAAGTATCACCAGTTCCGTTTC
CTCTGCCATTCAAATGCCCTACCTAGCCACGTGAAGATCAGCGTTTCCAGGCAGACGCTT
TTCGAAGATTCCTTCCAACAGATCATGAACATGAAACCCTATGACCTGCGCCGCCGGCTT
TACATCATCATGCGTGGCGAGGAGGGCCTGGACTATGGGGGCATCGCCAGAGAGTGGTTT
TTCCTCCTGTCTCACGAGGTGCTCAACCCTATGTATTGTTTATTTGAATATGCCGGAAAG
AACAATTACTGCCTGCAGATCAACCCCGCCTCCTCCATCAACCCGGACCACCTCACCTAC
TTTCGCTTTATAGGCAGATTCATCGCCATGGCGCTGTACCATGGAAAGTTCATCGACACG
GGCTTCACCCTCCCTTTCTACAAGCGGATGCTCAATAAGAGACCAACCCTGAAAGACCTG
GAGTCCATTGACCCTGAGTTCTACAACTCCATTGTCTGGATCAAAGAGAACAACCTGGAA
GAATGTGGCCTGGAGCTGTACTTCATCCAGGACATGGAGATACTGGGCAAGGTGACGACC
CACGAGCTGAAGGAGGGCGGCGAGAGCATCCGGGTCACGGAGGAGAACAAGGAAGAGTAC
ATCATGCTGCTGACTGACTGGCGTTTCACCCGAGGCGTGGAAGAGCAGACCAAAGCCTTC
CTGGATGGCTTCAACGAGGTGGCCCCGCTGGAGTGGCTGCGCTACTTTGACGAGAAAGAG
CTGGAGCTGATGCTGTGCGGCATGCAGGAGATAGACATGAGCGACTGGCAGAAGAGCACC
ATCTACCGGCACTACACCAAGAACAGCAAGCAGATCCAGTGGTTCTGGCAGGTGGTGAAG
GAGATGGACAACGAGAAGAGGATCCGGCTGCTGCAGTTTGTCACCGGTACCTGCCGCCTG
CCCGTCGGGGGATTTGCCGAACTCATCGGTAGCAACGGACCACAGAAGTTTTGCATTGAC
AAAGTTGGCAAGGAAACCTGGCTGCCCAGAAGCCACACCTGCTTCAACCGTCTGGATCTT
CCACCCTACAAGAGCTACGAACAGCTGAGAGAGAAGCTGCTGTATGCCATTGAGGAGACC
GAGGGCTTTGGACAGGAGTAACCGAGGCCGCCCCTCCCACGCCCCCCAGCGCACATGTAG
TCCTGAGTCCTCCCTGCCTGAGAGGCCACTGGCCCCGCAGCCCTTGGGAGGCCCCCGTGG
ATGTGGCCCTGTGTGGGACCACACTGTCATCTCGCTGCTGGCAGAAAAGCCTGATCCCAG
GAGGCCCTGCAGTTCCCCCGACCCGCGGATGGCAGTCTGGAATAAAGCCCCCTAGTTGCC
TTTGGCCCCACCTTTGCAAAGTTCCAGAGGGCTGACCCTCTCTGCAAAACTCTCCCCTGT
CCTCTAGACCCCACCCTGGGTGTATGTGAGTGTGCAAGGGAAGGTGTTGCATCCCCAGGG
GCTGCCGCAGAGGCCGGAGACCTCCTGGACTAGTTCGGCGAGGAGACTGGCCACTGGGGG
TGGCTGTTCGGGACTGAGAGCGCCAAGGGTCTTTGCCAGCAAAGGAGGTTCTGCCTGTAA
TTGAGCCTCTCTGATGATGGAGATGAAGTGAAGGTCTGAGGGACGGGCCCTGGGGCTAGG
CCATCTCTGCCTGCCTCCCTAGCAGGCGCCAGCGGTGGAGGCTGAGTCGCAGGACACATG
CCGGCCAGTTAATTCATTCTCAGCAAATGAAGGTTTGTCTAAGCTGCCTGGGTATCCACG
GGACAAAAACAGCAAACTCCCTCCAGACTTTGTCCATGTTATAAACTTGAAAGTTGGTTG
TTGTTTGTTAGGTTTGCCAGGTTTTTTTGTTTACGCCTGCTGTCACTTTCCTGTC
Human WWP2 nucleic acid sequence
(uniprot.org/uniprot/000308).
(SEQ ID NO: 49)
GAATTCGCGGCCGCGTCGACCGCTTCTGTGGCCACGGCAGATGAAACAGAAAGGCTAAAG
AGGGCTGGAGTCAGGGGACTTCTCTTCCACCAGCTTCACGGTGATGATATGGCATCTGCC
AGCTCTAGCCGGGCAGGAGTGGCCCTGCCTTTTGAGAAGTCTCAGCTCACTTTGAAAGTG
GTGTCCGCAAAGCCCAAGGTGCATAATCGTCAACCTCGAATTAACTCCTACGTGGAGGTG
GCGGTGGATGGACTCCCCAGTGAGACCAAGAAGACTGGGAAGCGCATTGGGAGCTCTGAG
CTTCTCTGGAATGAGATCATCATTTTGAATGTCACGGCACAGAGTCATTTAGATTTAAAG
GTCTGGAGCTGCCATACCTTGAGAAATGAACTGCTAGGCACCGCATCTGTCAACCTCTCC
AACGTCTTGAAGAACAATGGGGGCAAAATGGAGAACATGCAGCTGACCCTGAACCTGCAG
ACGGAGAACAAAGGCAGCGTTGTCTCAGGCGGAAAACTGACAATTTTCCTGGACGGGCCA
ACTGTTGATCTGGGAAATGTGCCTAATGGCAGTGCCCTGACAGATGGATCACAGCTGCCT
TCGAGAGACTCCAGTGGAACAGCAGTAGCTCCAGAGAACCGGCACCAGCCCCCCAGCACA
AACTGCTTTGGTGGAAGATCCCGGACGCACAGACATTCGGGTGCTTCAGCCAGAACAACC
CCAGCAACCGGCGAGCAAAGCCCCGGTGCTCGGAGCCGGCACCGCCAGCCCGTCAAGAAC
TCAGGCCACAGTGGCTTGGCCAATGGCACAGTGAATGATGAACCCACAACAGCCACTGAT
CCCGAAGAACCTTCCGTTGTTGGTGTGACGTCCCCACCTGCTGCACCCTTGAGTGTGACC
CCGAATCCCAACACGACTTCTCTCCCTGCCCCAGCCACACCGGCTGAAGGAGAGGAACCC
AGCACTTCGGGTACACAGCAGCTCCCAGCGGCTGCCCAGGCCCCCGACGCTCTGCCTGCT
GGATGGGAACAGCGAGAGCTGCCCAACGGACGTGTCTATTATGTTGACCACAATACCAAG
ACCACCACCTGGGAGCGGCCCCTTCCTCCAGGCTGGGAAAAACGCACAGATCCCCGAGGC
AGGTTTTACTATGTGGATCACAATACTCGGACCACCACCTGGCAGCGTCCGACCGCGGAG
TACGTGCGCAACTATGAGCAGTGGCAGTCGCAGCGGAATCAGCTCCAGGGGGCCATGCAG
CACTTCAGCCAAAGATTCCTATACCAGTTTTGGAGTGCTTCGACTGACCATGATCCCCTG
GGCCCCCTCCCTCCTGGTTGGGAGAAAAGACAGGACAATGGACGGGTGTATTACGTGAAC
CATAACACTCGCACGACCCAGTGGGAGGATCCCCGGACCCAGGGGATGATCCAGGAACCA
GCTTTGCCCCCAGGATGGGAGATGAAATACACCAGCGAGGGGGTGCGATACTTTGTGGAC
CACAATACCCGCACCACCACCTTTAAGGATCCTCGCCCGGGGTTTGAGTCGGGGACGAAG
CAAGGTTCCCCTGGTGCTTATGACCGCAGTTTTCGGTGGAAGTATCACCAGTTCCGTTTC
CTCTGCCATTCAAATGCCCTACCTAGCCACGTGAAGATCAGCGTTTCCAGGCAGACGCTT
TTCGAAGATTCCTTCCAACAGATCATGAACATGAAACCCTATGACCTGCGCCGCCGGCTT
TACATCATCATGCGTGGCGAGGAGGGCCTGGACTATGGGGGCATCGCCAGAGAGTGGTTT
TTCCTCCTGTCTCACGAGGTGCTCAACCCTATGTATTGTTTATTTGAATATGCCGGAAAG
AACAATTACTGCCTGCAGATCAACCCCGCCTCCTCCATCAACCCGGACCACCTCACCTAC
TTTCGCTTTATAGGCAGATTCATCGCCATGGCGCTGTACCATGGAAAGTTCATCGACACG
GGCTTCACCCTCCCTTTCTACAAGCGGATGCTCAATAAGAGACCAACCCTGAAAGACCTG
GAGTCCATTGACCCTGAGTTCTACAACTCCATTGTCTGGATCAAAGAGAACAACCTGGAA
GAATGTGGCCTGGAGCTGTACTTCATCCAGGACATGGAGATACTGGGCAAGGTGACGACC
CACGAGCTGAAGGAGGGCGGCGAGAGCATCCGGGTCACGGAGGAGAACAAGGAAGAGTAC
ATCATGCTGCTGACTGACTGGCGTTTCACCCGAGGCGTGGAAGAGCAGACCAAAGCCTTC
CTGGATGGCTTCAACGAGGTGGCCCCGCTGGAGTGGCTGCGCTACTTTGACGAGAAAGAG
CTGGAGCTGATGCTGTGCGGCATGCAGGAGATAGACATGAGCGACTGGCAGAAGAGCACC
ATCTACCGGCACTACACCAAGAACAGCAAGCAGATCCAGTGGTTCTGGCAGGTGGTGAAG
GAGATGGACAACGAGAAGAGGATCCGGCTGCTGCAGTTTGTCACCGGTACCTGCCGCCTG
CCCGTCGGGGGATTTGCCGAACTCATCGGTAGCAACGGACCACAGAAGTTTTGCATTGAC
AAAGTTGGCAAGGAAACCTGGCTGCCCAGAAGCCACACCTGCTTCAACCGTCTGGATCTT
CCACCCTACAAGAGCTACGAACAGCTGAGAGAGAAGCTGCTGTATGCCATTGAGGAGACC
GAGGGCTTTGGACAGGAGTAACCGAGGCCGCCCCTCCCACGCCCCCCAGCGCACATGTAG
TCCTGAGTCCTCCCTGCCTGAGAGGCCACTGGCCCCGCAGCCCTTGGGAGGCCCCCGTGG
ATGTGGCCCTGTGTGGGACCACACTGTCATCTCGCTGCTGGCAGAAAAGCCTGATCCCAG
GAGGCCCTGCAGTTCCCCCGACCCGCGGATGGCAGTCTGGAATAAAGCCCCCTAGTTGCC
TTTGGCCCCACCTTTGCAAAGTTCCAGAGGGCTGACCCTCTCTGCAAAACTCTCCCCTGT
CCTCTAGACCCCACCCTGGGTGTATGTGAGTGTGCAAGGGAAGGTGTTGCATCCCCAGGG
GCTGCCGCAGAGGCCGGAGACCTCCTGGACTAGTTCGGCGAGGAGACTGGCCACTGGGGG
TGGCTGTTCGGGACTGAGAGCGCCAAGGGTCTTTGCCAGCAAAGGAGGTTCTGCCTGTAA
TTGAGCCTCTCTGATGATGGAGATGAAGTGAAGGTCTGAGGGACGGGCCCTGGGGCTAGG
CCATCTCTGCCTGCCTCCCTAGCAGGCGCCAGCGGTGGAGGCTGAGTCGCAGGACACATG
CCGGCCAGTTAATTCATTCTCAGCAAATGAAGGTTTGTCTAAGCTGCCTGGGTATCCACG
GGACAAAAACAGCAAACTCCCTCCAGACTTTGTCCATGTTATAAACTTGAAAGTTGGTTG
TTGTTTGTTAGGTTTGCCAGGTTTTTTTGTTTACGCCTGCTGTCACTTTCCTGTC
Human Nedd4-1 nucleic acid sequence
(uniprot.org/uniprot/P46934).
(SEQ ID NO: 50)
ACAGTTGCCTGCCCTGGGGGGGGCGAGCGCGTCCGGTTTGCTGGAAGCGTTCGGAAATG
GCAACTTGCGCGGTGGAGGTGTTCGGGCTCCTGGAGGACGAGGAAAATTCACGAATTGTG
AGAGTAAGAGTTATAGCCGGAATAGGCCTTGCCAAGAAGGATATATTGGGAGCTAGTGAT
CCTTACGTGAGAGTGACGTTATATGACCCAATGAATGGAGTTCTTACAAGTGTGCAAACA
AAAACCATTAAAAAGAGTTTGAATCCAAAGTGGAATGAAGAAATATTATTCAGAGTTCAT
CCTCAGCAGCACCGGCTTCTTTTTGAAGTGTTTGACGAAAACCGATTGACAAGAGATGAT
TTCCTAGGTCAAGTGGATGTTCCACTTTATCCATTACCGACAGAAAATCCAAGATTGGAG
AGACCATATACATTTAAGGATTTTGTTCTTCATCCAAGAAGTCACAAATCAAGAGTTAAA
GGTTATCTGAGACTAAAAATGACTTATTTACCTAAAACCAGTGGCTCAGAAGATGATAAT
GCAGAACAGGCTGAGGAATTAGAGCCTGGCTGGGTTGTTTTGGACCAACCAGATGCTGCT
TGCCATTTGCAGCAACAACAAGAACCTTCTCCTCTACCTCCAGGGTGGGAAGAGAGGCAG
GATATCCTTGGAAGGACCTATTATGTAAACCATGAATCTAGAAGAACACAGTGGAAAAGA
CCAACCCCTCAGGACAACCTAACAGATGCTGAGAATGGCAACATTCAACTGCAAGCACAA
CGTGCATTTACCACCAGGCGGCAGATATCCGAGGAAACAGAAAGTGTTGACAACCAAGAG
TCTTCCGAGAACTGGGAAATTATAAGAGAAGATGAAGCCACCATGTATAGCAGCCAGGCC
TTCCCATCACCTCCACCGTCAAGTAACTTGGATGTTCCAACTCATCTTGCAGAAGAATTG
AATGCCAGACTCACCATTTTTGGAAATTCAGCCGTGAGCCAGCCAGCATCGAGCTCAAAT
CATTCCAGCAGAAGAGGCAGCTTACAAGCCTATACTTTTGAGGAACAACCTACACTTCCT
GTGCTTTTGCCTACTTCATCTGGATTACCACCAGGTTGGGAAGAAAAACAAGATGAAAGA
GGAAGATCATATTATGTAGATCACAATTCCAGAACGACTACTTGGACAAAGCCCACTGTA
CAGGCCACAGTGGAGACCAGTCAGCTGACCTCAAGCCAGAGTTCTGCAGGCCCTCAATCA
CAAGCCTCCACCAGTGATTCAGGCCAGCAGGTGACCCAGCCATCTGAAATTGAGCAAGGA
TTCCTTCCTAAAGGCTGGGAAGTCCGGCATGCACCAAATGGGAGGCCTTTCTTTATTGAC
CACAACACTAAAACCACCACCTGGGAAGATCCAAGATTGAAAATTCCAGCCCATCTGAGA
GGAAAGACATCACTTGATACTTCCAATGATCTAGGGCCTTTACCTCCAGGATGGGAAGAG
AGAACTCACACAGATGGAAGAATCTTCTACATAAATCACAATATAAAAAGAACACAATGG
GAAGATCCTCGGTTGGAGAATGTAGCAATAACTGGACCAGCAGTGCCCTACTCCAGGGAT
TACAAAAGAAAGTATGAGTTCTTCCGAAGAAAGTTGAAGAAGCAGAATGACATTCCAAAC
AAATTTGAAATGAAACTTCGCCGAGCAACTGTTCTTGAAGACTCTTACCGGAGAATTATG
GGTGTCAAGAGAGCAGACTTCCTGAAGGCTCGACTGTGGATTGAGTTTGATGGTGAAAAG
GGATTGGATTATGGAGGAGTTGCCAGAGAATGGTTCTTCCTGATCTCAAAGGAAATGTTT
AACCCTTATTATGGGTTGTTTGAATATTCTGCTACGGACAATTATACCCTACAGATAAAT
CCAAACTCTGGATTGTGTAACGAAGATCACCTCTCTTACTTCAAGTTTATTGGTCGGGTA
GCTGGAATGGCAGTTTATCATGGCAAACTGTTGGATGGTTTTTTCATCCGCCCATTTTAC
AAGATGATGCTTCACAAACCAATAACCCTTCATGATATGGAATCTGTGGATAGTGAATAT
TACAATTCCCTAAGATGGATTCTTGAAAATGACCCAACAGAATTGGACCTCAGGTTTATC
ATAGATGAAGAACTTTTTGGACAGACACATCAACATGAGCTGAAAAATGGTGGATCAGAA
ATAGTTGTCACCAATAAGAACAAAAAGGAATATATTTATCTTGTAATACAATGGCGATTT
GTAAACCGAATCCAGAAGCAAATGGCTGCTTTTAAAGAGGGATTCTTTGAACTAATACCA
CAGGATCTCATCAAAATTTTTGATGAAAATGAACTAGAGCTTCTTATGTGTGGACCGGGA
GATGTTGATGTGAATGACTGGAGGGAACATACAAAGTATAAAAATGGCTACAGTGCAAAT
CATCAGGTTATACAGTGGTTTTGGAAGGCTGTTTTAATGATGGATTCAGAAAAAAGAATA
AGATTACTTCAGTTTGTCACTGGCACATCTCGGGTGCCTATGAATGGATTTGCTGAACTA
TACGGTTCAAATGGACCACAGTCATTTACAGTTGAACAGTGGGGTACTCCTGAAAAGCTG
CCAAGAGCTCATACCTGTTTTAATCGCCTGGACTTGCCACCTTATGAATCATTTGAAGAA
TTATGGGATAAACTTCAGATGGCAATTGAAAACACCCAGGGCTTTGATGGAGTTGATTAG
ATTACAAATAACAATCTGTAGTGTTTTTACTGCCATAGTTTTATAACCAAAATCTTGACT
TAAAATTTTCCGGGGAACTACTAAAATGTGGCCACTGAGTCTTCCCAGATCTTGAAGAAA
ATCATATAAAAAGCATTTGAAGAAATAGTACGAC

Human Nedd4-2 nucleic acid sequence (>gi|345478679|ref|NM_015277.5| Homo sapiens neural precursor cell expressed, developmentally down-regulated 4-like, E3 ubiquitin protein ligase (NEDD4L), transcript variant d, mRNA).

(SEQ ID NO: 51)
ATGGCGACCGGGCTCGGGGAGCCGGTCTATGGACTTTCCGAAGACGAGGGAGAGTCCCGTAT
TCTCAGAGTAAAAGTTGTTTCTGGAATTGATCTCGCCAAAAAGGACATCTTTGGAGCCAGTG
ATCCGTATGTGAAACTTTCATTGTACGTAGCGGATGAGAATAGAGAACTTGCTTTGGTCCAG
ACAAAAACAATTAAAAAGACACTGAACCCAAAATGGAATGAAGAATTTTATTTCAGGGTAAA
CCCATCTAATCACAGACTCCTATTTGAAGTATTTGACGAAAATAGACTGACACGAGACGACT
TCCTGGGCCAGGTGGACGTGCCCCTTAGTCACCTTCCGACAGAAGATCCAACCATGGAGCGA
CCCTATACATTTAAGGACTTTCTCCTCAGACCAAGAAGTCATAAGTCTCGAGTTAAGGGATT
TTTGCGATTGAAAATGGCCTATATGCCAAAAAATGGAGGTCAAGATGAAGAAAACAGTGACC
AGAGGGATGACATGGAGCATGGATGGGAAGTTGTTGACTCAAATGACTCGGCTTCTCAGCAC
CAAGAGGAACTTCCTCCTCCTCCTCTGCCTCCCGGGTGGGAAGAAAAAGTGGACAATTTAGG
CCGAACTTACTATGTCAACCACAACAACCGGACCACTCAGTGGCACAGACCAAGCCTGATGG
ACGTGTCCTCGGAGTCGGACAATAACATCAGACAGATCAACCAGGAGGCAGCACACCGGCGC
TTCCGCTCCCGCAGGCACATCAGCGAAGACTTGGAGCCCGAGCCCTCGGAGGGCGGGGATGT
CCCCGAGCCTTGGGAGACCATTTCAGAGGAAGTGAATATCGCTGGAGACTCTCTCGGTCTGG
CTCTGCCCCCACCACCGGCCTCCCCAGGATCTCGGACCAGCCCTCAGGAGCTGTCAGAGGAA
CTAAGCAGAAGGCTTCAGATCACTCCAGACTCCAATGGGGAACAGTTCAGCTCTTTGATTCA
AAGAGAACCCTCCTCAAGGTTGAGGTCATGCAGTGTCACCGACGCAGTTGCAGAACAGGGCC
ATCTACCACCGCCATCAGTGGCCTATGTACATACCACGCCGGGTCTGCCTTCAGGCTGGGAA
GAAAGAAAAGATGCTAAGGGGCGCACATACTATGTCAATCATAACAATCGAACCACAACTTG
GACTCGACCTATCATGCAGCTTGCAGAAGATGGTGCGTCCGGATCAGCCACAAACAGTAACA
ACCATCTAATCGAGCCTCAGATCCGCCGGCCTCGTAGCCTCAGCTCGCCAACAGTAACTTTA
TCTGCCCCGCTGGAGGGTGCCAAGGACTCACCCGTACGTCGGGCTGTGAAAGACACCCTTTC
CAACCCACAGTCCCCACAGCCATCACCTTACAACTCCCCCAAACCACAACACAAAGTCACAC
AGAGCTTCTTGCCACCCGGCTGGGAAATGAGGATAGCGCCAAACGGCCGGCCCTTCTTCATT
GATCATAACACAAAGACTACAACCTGGGAAGATCCACGTTTGAAATTTCCAGTACATATGCG
GTCAAAGACATCTTTAAACCCCAATGACCTTGGCCCCCTTCCTCCTGGCTGGGAAGAAAGAA
TTCACTTGGATGGCCGAACGTTTTATATTGATCATAATAGCAAAATTACTCAGTGGGAAGAC
CCAAGACTGCAGAACCCAGCTATTACTGGTCCGGCTGTCCCTTACTCCAGAGAATTTAAGCA
GAAATATGACTACTTCAGGAAGAAATTAAAGAAACCTGCTGATATCCCCAATAGGTTTGAAA
TGAAACTTCACAGAAATAACATATTTGAAGAGTCCTATCGGAGAATTATGTCCGTGAAAAGA
CCAGATGTCCTAAAAGCTAGACTGTGGATTGAGTTTGAATCAGAGAAAGGTCTTGACTATGG
GGGTGTGGCCAGAGAATGGTTCTTCTTACTGTCCAAAGAGATGTTCAACCCCTACTACGGCC
TCTTTGAGTACTCTGCCACGGACAACTACACCCTTCAGATCAACCCTAATTCAGGCCTCTGI
AATGAGGATCATTTGTCCTACTTCACTTTTATTGGAAGAGTTGCTGGTCTGGCCGTATTTCA
TGGGAAGCTCTTAGATGGTTTCTTCATTAGACCATTTTACAAGATGATGTTGGGAAAGCAGA
TAACCCTGAATGACATGGAATCTGTGGATAGTGAATATTACAACTCTTTGAAATGGATCCTG
GAGAATGACCCTACTGAGCTGGACCTCATGTTCTGCATAGACGAAGAAAACTTTGGACAGAC
ATATCAAGTGGATTTGAAGCCCAATGGGTCAGAAATAATGGTCACAAATGAAAACAAAAGGG
AATATATCGACTTAGTCATCCAGTGGAGATTTGTGAACAGGGTCCAGAAGCAGATGAACGCC
TTCTTGGAGGGATTCACAGAACTACTTCCTATTGATTTGATTAAAATTTTTGATGAAAATGA
GCTGGAGTTGCTCATGTGCGGCCTCGGTGATGTGGATGTGAATGACTGGAGACAGCATTCTA
TTTACAAGAACGGCTACTGCCCAAACCACCCCGTCATTCAGTGGTTCTGGAAGGCTGTGCTA
CTCATGGACGCCGAAAAGCGTATCCGGTTACTGCAGTTTGTCACAGGGACATCGCGAGTACC
TATGAATGGATTTGCCGAACTTTATGGTTCCAATGGTCCTCAGCTGTTTACAATAGAGCAAT
GGGGCAGTCCTGAGAAACTGCCCAGAGCTCACACATGCTTTAATCGCCTTGACTTACCTCCA
TATGAAACCTTTGAAGATTTACGAGAGAAACTTCTCATGGCCGTGGAAAATGCTCAAGGATT
TGAAGGGGTGGATTAA
Human Smurf1 nucleic acid sequence (uniprot.org/uniprot/Q9HCE7).
(SEQ ID NO: 52)
ATGTCGAACCCCGGGACACGCAGGAACGGCTCCAGCATCAAGATCCGTCTGACAGTGTTA
TGTGCCAAGAACCTTGCAAAGAAAGACTTCTTCAGGCTCCCTGACCCTTTTGCAAAGATT
GTCGTGGATGGGTCTGGGCAGTGCCACTCAACCGACACTGTGAAAAACACATTGGACCCA
AAGTGGAACCAGCACTATGATCTATATGTTGGGAAAACGGATTCGATAACCATTAGCGTG
TGGAACCATAAGAAAATTCACAAGAAACAGGGAGCTGGCTTCCTGGGCTGTGTGCGGCTG
CTCTCCAATGCCATCAGCAGATTAAAAGATACCGGATACCAGCGTTTGGATCTATGCAAA
CTAAACCCCTCAGATACTGATGCAGTTCGTGGCCAGATAGTGGTCAGTTTACAGACACGA
GACAGAATAGGAACCGGCGGCTCGGTGGTGGACTGCAGAGGACTGTTAGAAAATGAAGGA
ACGGTGTATGAAGACTCCGGGCCTGGGAGGCCGCTCAGCTGCTTCATGGAGGAACCAGCC
CCTTACACAGATAGCACCGGTGCTGCTGCTGGAGGAGGGAATTGCAGGTTCGTGGAGTCC
CCAAGTCAAGATCAAAGACTTCAGGCACAGCGGCTTCGAAACCCTGATGTGCGAGGTTCA
CTACAGACGCCCCAGAACCGACCACACGGCCACCAGTCCCCGGAACTGCCCGAAGGCTAC
GAACAAAGAACAACAGTCCAGGGCCAAGTTTACTTTTTGCATACACAGACTGGAGTTAGC
ACGTGGCACGACCCCAGGATACCAAGTCCCTCGGGGACCATTCCTGGGGGAGATGCAGCT
TTTCTATACGAATTCCTTCTACAAGGCCATACATCTGAGCCCAGAGACCTTAACAGTGTG
AACTGTGATGAACTTGGACCACTGCCGCCAGGCTGGGAAGTCAGAAGTACAGTTTCTGGG
AGGATATATTTTGTAGATCATAATAACCGAACAACCCAGTTTACAGACCCAAGGTTACAC
CACATCATGAATCACCAGTGCCAACTCAAGGAGCCCAGCCAGCCGCTGCCACTGCCCAGT
GAGGGCTCTCTGGAGGACGAGGAGCTTCCTGCCCAGAGATACGAAAGAGATCTAGTCCAG
AAGCTGAAAGTCCTCAGACACGAACTGTCGCTTCAGCAGCCCCAAGCTGGTCATTGCCGC
ATCGAAGTGTCCAGAGAAGAAATCTTTGAGGAGTCTTACCGCCAGATAATGAAGATGCGA
CCGAAAGACTTGAAAAAACGGCTGATGGTGAAATTCCGTGGGGAAGAAGGTTTGGATTAC
GGTGGTGTGGCCAGGGAGTGGCTTTACTTGCTGTGCCATGAAATGCTGAATCCTTATTAC
GGGCTCTTCCAGTATTCTACGGACAATATTTACATGTTGCAAATAAATCCGGATTCTTCA
ATCAACCCCGACCACTTGTCTTATTTCCACTTTGTGGGGCGGATCATGGGGCTGGCTGTG
TTCCATGGACACTACATCAACGGGGGCTTCACAGTGCCCTTCTACAAGCAGCTGCTGGGG
AAGCCCATCCAGCTCTCAGATCTGGAATCTGTGGACCCAGAGCTGCATAAGAGCTTGGTG
TGGATCCTAGAGAACGACATCACGCCTGTACTGGACCACACCTTCTGCGTGGAACACAAC
GCCTTCGGGCGGATCCTGCAGCATGAACTGAAACCCAATGGCAGAAATGTGCCAGTCACA
GAGGAGAATAAGAAAGAATACGTCCGGTTGTATGTAAACTGGAGGTTTATGAGAGGAATC
GAAGCCCAGTTCTTAGCTCTGCAGAAGGGGTTCAATGAGCTCATCCCTCAACATCTGCTG
AAGCCTTTTGACCAGAAGGAACTGGAGCTGATCATAGGCGGCCTGGATAAAATAGACTTG
AACGACTGGAAGTCGAACACGCGGCTGAAGCACTGTGTGGCCGACAGCAACATCGTGCGG
TGGTTCTGGCAAGCGGTGGAGACGTTCGATGAAGAAAGGAGGGCCAGGCTCCTGCAGTTT
GTGACTGGGTCCACGCGAGTCCCGCTCCAAGGCTTCAAGGCTTTGCAAGGTTCTACAGGC
GCGGCAGGGCCCCGGCTGTTCACCATCCACCTGATAGACGCGAACACAGACAACCTTCCG
AAGGCCCATACCTGCTTTAACCGGATCGACATTCCACCATATGAGTCCTATGAGAAGCTC
TACGAGAAGCTGCTGACAGCCGTGGAGGAGACCTGCGGGTTTGCTGTGGAGTGA
Human Smurf2 nucleic acid sequence (uniprot.org/uniprot/Q9HAU4).
(SEQ ID NO: 53)
ATGTCTAACCCCGGACGCCGGAGGAACGGGCCCGTCAAGCTGCGCCTGACAGTACTCTGT
GCAAAAAACCTGGTGAAAAAGGATTTTTTCCGACTTCCTGATCCATTTGCTAAGGTGGTG
GTTGATGGATCTGGGCAATGCCATTCTACAGATACTGTGAAGAATACGCTTGATCCAAAG
TGGAATCAGCATTATGACCTGTATATTGGAAAGTCTGATTCAGTTACGATCAGTGTATGG
AATCACAAGAAGATCCATAAGAAACAAGGTGCTGGATTTCTCGGTTGTGTTCGTCTTCTT
TCCAATGCCATCAACCGCCTCAAAGACACTGGTTATCAGAGGTTGGATTTATGCAAACTC
GGGCCAAATGACAATGATACAGTTAGAGGACAGATAGTAGTAAGTCTTCAGTCCAGAGAC
CGAATAGGCACAGGAGGACAAGTTGTGGACTGCAGTCGTTTATTTGATAACGATTTACCA
GACGGCTGGGAAGAAAGGAGAACCGCCTCTGGAAGAATCCAGTATCTAAACCATATAACA
AGAACTACGCAATGGGAGCGCCCAACACGACCGGCATCCGAATATTCTAGCCCTGGCAGA
CCTCTTAGCTGCTTTGTTGATGAGAACACTCCAATTAGTGGAACAAATGGTGCAACATGT
GGACAGTCTTCAGATCCCAGGCTGGCAGAGAGGAGAGTCAGGTCACAACGACATAGAAAT
TACATGAGCAGAACACATTTACATACTCCTCCAGACCTACCAGAAGGCTATGAACAGAGG
ACAACGCAACAAGGCCAGGTGTATTTCTTACATACACAGACTGGTGTGAGCACATGGCAT
GATCCAAGAGTGCCCAGGGATCTTAGCAACATCAATTGTGAAGAGCTTGGTCCATTGCCT
CCTGGATGGGAGATCCGTAATACGGCAACAGGCAGAGTTTATTTCGTTGACCATAACAAC
AGAACAACACAATTTACAGATCCTCGGCTGTCTGCTAACTTGCATTTAGTTTTAAATCGG
CAGAACCAATTGAAAGACCAACAGCAACAGCAAGTGGTATCGTTATGTCCTGATGACACA
GAATGCCTGACAGTCCCAAGGTACAAGCGAGACCTGGTTCAGAAACTAAAAATTTTGCGG
CAAGAACTTTCCCAACAACAGCCTCAGGCAGGTCATTGCCGCATTGAGGTTTCCAGGGAA
GAGATTTTTGAGGAATCATATCGACAGGTCATGAAAATGAGACCAAAAGATCTCTGGAAG
CGATTAATGATAAAATTTCGTGGAGAAGAAGGCCTTGACTATGGAGGCGTTGCCAGGGAA
TGGTTGTATCTCTTGTCACATGAAATGTTGAATCCATACTATGGCCTCTTCCAGTATTCA
AGAGATGATATTTATACATTGCAGATCAATCCTGATTCTGCAGTTAATCCGGAACATTTA
TCCTATTTCCACTTTGTTGGACGAATAATGGGAATGGCTGTGTTTCATGGACATTATATT
GATGGTGGTTTCACATTGCCTTTTTATAAGCAATTGCTTGGGAAGTCAATTACCTTGGAT
GACATGGAGTTAGTAGATCCGGATCTTCACAACAGTTTAGTGTGGATACTTGAGAATGAT
ATTACAGGTGTTTTGGACCATACCTTCTGTGTTGAACATAATGCATATGGTGAAATTATT
CAGCATGAACTTAAACCAAATGGCAAAAGTATCCCTGTTAATGAAGAAAATAAAAAAGAA
TATGTCAGGCTCTATGTGAACTGGAGATTTTTACGAGGCATTGAGGCTCAATTCTTGGCT
CTGCAGAAAGGATTTAATGAAGTAATTCCACAACATCTGCTGAAGACATTTGATGAGAAG
GAGTTAGAGCTCATTATTTGTGGACTTGGAAAGATAGATGTTAATGACTGGAAGGTAAAC
ACCCGGTTAAAACACTGTACACCAGACAGCAACATTGTCAAATGGTTCTGGAAAGCTGTG
GAGTTTTTTGATGAAGAGCGACGAGCAAGATTGCTTCAGTTTGTGACAGGATCCTCTCGA
GTGCCTCTGCAGGGCTTCAAAGCATTGCAAGGTGCTGCAGGCCCGAGACTCTTTACCATA
CACCAGATTGATGCCTGCACTAACAACCTGCCGAAAGCCCACACTTGCTTCAATCGAATA
GACATTCCACCCTATGAAAGCTATGAAAAGCTATATGAAAAGCTGCTAACAGCCATTGAA
GAAACATGTGGATTTGCTGTGGAATGA
Human ITCH nucleic acid sequence (uniprot.org/uniprot/Q96J02).
(SEQ ID NO: 54)
GGAGTCGCCGCCGCCCCGAGTTCCGGTACCATGCATTTCACGGTGGCCTTGTGGAGACAA
CGCCTTAACCCAAGGAAGTGACTCAAACTGTGAGAACTCCAGGTTTTCCAACCTATTGGT
GGTATGTCTGACAGTGGATCACAACTTGGTTCAATGGGTAGCCTCACCATGAAATCACAG
CTTCAGATCACTGTCATCTCAGCAAAACTTAAGGAAAATAAGAAGAATTGGTTTGGACCA
AGTCCTTACGTAGAGGTCACAGTAGATGGACAGTCAAAGAAGACAGAAAAATGCAACAAC
ACAAACAGTCCCAAGTGGAAGCAACCCCTTACAGTTATCGTTACCCCTGTGAGTAAATTA
CATTTTCGTGTGTGGAGTCACCAGACACTGAAATCTGATGTTTTGTTGGGAACTGCTGCA
TTAGATATTTATGAAACATTAAAGTCAAACAATATGAAACTTGAAGAAGTAGTTGTGACT
TTGCAGCTTGGAGGTGACAAAGAGCCAACAGAGACAATAGGAGACTTGTCAATTTGTCTT
GATGGGCTACAGTTAGAGTCTGAAGTTGTTACCAATGGTGAAACTACATGTTCAGAAAGT
GCTTCTCAGAATGATGATGGCTCCAGATCCAAGGATGAAACAAGAGTGAGCACAAATGGA
TCAGATGACCCTGAAGATGCAGGAGCTGGTGAAAATAGGAGAGTCAGTGGGAATAATTCT
CCATCACTCTCAAATGGTGGTTTTAAACCTTCTAGACCTCCAAGACCTTCACGACCACCA
CCACCCACCCCACGTAGACCAGCATCTGTCAATGGTTCACCATCTGCCACTTCTGAAAGT
GATGGGTCTAGTACAGGCTCTCTGCCGCCGACAAATACAAATACAAATACATCTGAAGGA
GCAACATCTGGATTAATAATTCCTCTTACTATATCTGGAGGCTCAGGCCCTAGGCCATTA
AATCCTGTAACTCAAGCTCCCTTGCCACCTGGTTGGGAGCAGAGAGTGGACCAGCACGGG
CGAGTTTACTATGTAGATCATGTTGAGAAAAGAACAACATGGGATAGACCAGAACCTCTA
CCTCCTGGCTGGGAACGGCGGGTTGACAACATGGGACGTATTTATTATGTTGACCATTTC
ACAAGAACAACAACGTGGCAGAGGCCAACACTGGAATCCGTCCGGAACTATGAACAATGG
CAGCTACAGCGTAGTCAGCTTCAAGGAGCAATGCAGCAGTTTAACCAGAGATTCATTTAT
GGGAATCAAGATTTATTTGCTACATCACAAAGTAAAGAATTTGATCCTCTTGGTCCATTG
CCACCTGGATGGGAGAAGAGAACAGACAGCAATGGCAGAGTATATTTCGTCAACCACAAC
ACACGAATTACACAATGGGAAGACCCCAGAAGTCAAGGTCAATTAAATGAAAAGCCCTTA
CCTGAAGGTTGGGAAATGAGATTCACAGTGGATGGAATTCCATATTTTGTGGACCACAAT
AGAAGAACTACCACCTATATAGATCCCCGCACAGGAAAATCTGCCCTAGACAATGGACCT
CAGATAGCCTATGTTCGGGACTTCAAAGCAAAGGTTCAGTATTTCCGGTTCTGGTGTCAG
CAACTGGCCATGCCACAGCACATAAAGATTACAGTGACAAGAAAAACATTGTTTGAGGAT
TCCTTTCAACAGATAATGAGCTTCAGTCCCCAAGATCTGCGAAGACGTTTGTGGGTGATT
TTTCCAGGAGAAGAAGGTTTAGATTATGGAGGTGTAGCAAGAGAATGGTTCTTTCTTTTG
TCACATGAAGTGTTGAACCCAATGTATTGCCTGTTTGAATATGCAGGGAAGGATAACTAC
TGCTTGCAGATAAACCCCGCTTCTTACATCAATCCAGATCACCTGAAATATTTTCGTTTT
ATTGGCAGATTTATTGCCATGGCTCTGTTCCATGGGAAATTCATAGACACGGGTTTTTCT
TTACCATTCTATAAGCGTATCTTGAACAAACCAGTTGGACTCAAGGATTTAGAATCTATT
GATCCAGAATTTTACAATTCTCTCATCTGGGTTAAGGAAAACAATATTGAGGAATGTGAT
TTGGAAATGTACTTCTCCGTTGACAAAGAAATTCTAGGTGAAATTAAGAGTCATGATCTG
AAACCTAATGGTGGCAATATTCTTGTAACAGAAGAAAATAAAGAGGAATACATCAGAATG
GTAGCTGAGTGGAGGTTGTCTCGAGGTGTTGAAGAACAGACACAAGCTTTCTTTGAAGGC
TTTAATGAAATTCTTCCCCAGCAATATTTGCAATACTTTGATGCAAAGGAATTAGAGGTC
CTTTTATGTGGAATGCAAGAGATTGATTTGAATGACTGGCAAAGACATGCCATCTACCGT
CATTATGCAAGGACCAGCAAACAAATCATGTGGTTTTGGCAGTTTGTTAAAGAAATTGAT
AATGAGAAGAGAATGAGACTTCTGCAGTTTGTTACTGGAACCTGCCGATTGCCAGTAGGA
GGATTTGCTGATCTCATGGGGAGCAATGGACCACAGAAATTCTGCATTGAAAAAGTTGGG
AAAGAAAATTGGCTACCCAGAAGTCATACCTGTTTTAATCGCCTGGACCTGCCACCATAC
AAGAGCTATGAGCAACTGAAGGAAAAGCTGTTGTTTGCCATAGAAGAAACAGAAGGATTT
GGACAAGAGTAACTTCTGAGAACTTGCACCATGAATGGGCAAGAACTTATTTGCAATGTT
TGTCCTTCTCTGCCTGTTGCACATCTTGTAAAATTGGACAATGGCTCTTTAGAGAGTTAT
CTGAGTGTAAGTAAATTAATGTTCTCATTTAAAAAAAAAAAAAAAAAAA
Human NEDLI nucleic acid sequence (uniprot.org/uniprot/Q76N89).
(SEQ ID NO: 55)
GCGCATCAGGCGCTGTTGTTGGAGCCGGAACACCGTGCGACTCTGACCGAACCGGCCCCC
TCCTCGCGCACACACTCGCCGAGCCGCGCGCGCCCCTCCGCCGTGACAGTGGCCGTGGCC
TCCGCTCTCTCGGGGCACCCGGCAGCCAGAGCGCAGCGAGAGCGGGCGGTCGCCAGGGTC
CCCTCCCCAGCCAGTCCCAGGCGCCCGGTGCACTATGCGGGGCACGTGCGCCCCCCAGCT
CTAATCTGCGCGCTGACAGGAGCATGATCTGTGCCCAGGCCAGGGCTGCCAAGGAATTGA
TGCGCGTACACGTGGTGGGTCATTATGCTGCTACACCTGTGTAGTGTGAAGAATCTGTAC
CAGAACAGGTTTTTAGGCCTGGCCGCCATGGCGTCTCCTTCTAGAAACTCCCAGAGCCGA
CGCCGGTGCAAGGAGCCGCTCCGATACAGCTACAACCCCGACCAGTTCCACAACATGGAC
CTCAGGGGCGGCCCCCACGATGGCGTCACCATTCCCCGCTCCACCAGCGACACTGACCTG
GTCACCTCGGACAGCCGCTCCACGCTCATGGTCAGCAGCTCCTACTATTCCATCGGGCAC
TCTCAGGACCTGGTCATCCACTGGGACATAAAGGAGGAAGTGGACGCTGGGGACTGGATT
GGCATGTACCTCATTGATGAGGTCTTGTCCGAAAACTTTCTGGACTATAAAAACCGTGGA
GTCAATGGTTCTCATCGGGGCCAGATCATCTGGAAGATCGATGCCAGCTCGTACTTTGTG
GAACCTGAAACTAAGATCTGCTTCAAATACTACCATGGAGTGAGTGGGGCCCTGCGAGCA
ACCACCCCCAGTGTCACGGTCAAAAACTCGGCAGCTCCTATTTTTAAAAGCATTGGTGCT
GATGAGACCGTCCAAGGACAAGGAAGTCGGAGGCTGATCAGCTTCTCTCTCTCAGATTTC
CAAGCCATGGGGTTGAAGAAAGGGATGTTTTTCAACCCAGACCCTTATCTGAAGATTTCC
ATTCAGCCTGGGAAACACAGCATCTTCCCCGCCCTCCCTCACCATGGACAGGAGAGGAGA
TCCAAGATCATAGGCAACACCGTGAACCCCATCTGGCAGGCCGAGCAATTCAGTTTTGTG
TCCTTGCCCACTGACGTGCTGGAAATTGAGGTGAAGGACAAGTTTGCCAAGAGCCGCCCC
ATCATCAAGCGCTTCTTGGGAAAGCTGTCGATGCCCGTTCAAAGACTCCTGGAGAGACAC
GCCATAGGGGATAGGGTGGTCAGCTACACACTTGGCCGCAGGCTTCCAACAGATCATGTG
AGTGGACAGCTGCAATTCCGATTTGAGATCACTTCCTCCATCCACCCAGATGATGAGGAG
ATTTCCCTGAGTACCGAGCCTGAGTCAGCCCAAATTCAGGACAGCCCCATGAACAACCTG
ATGGAAAGCGGCAGTGGGGAACCTCGGTCTGAGGCACCAGAGTCCTCTGAGAGCTGGAAG
CCAGAGCAGCTGGGTGAGGGCAGTGTCCCCGATGGTCCAGGGAACCAAAGCATAGAGCTT
TCCAGACCAGCTGAGGAAGCAGCAGTCATCACGGAGGCAGGAGACCAGGGCATGGTCTCT
GTGGGACCTGAAGGGGCTGGGGAGCTCCTGGCCCAGGTGCAAAAGGACATCCAGCCTGCC
CCCAGTGCAGAAGAGCTGGCCGAGCAGCTGGACCTGGGTGAGGAGGCATCAGCACTGCTG
CTGGAAGACGGTGAAGCCCCAGCCAGCACCAAGGAGGAGCCCTTGGAGGAGGAAGCAACG
ACCCAGAGCCGGGCTGGAAGGGAAGAAGAGGAGAAGGAGCAGGAGGAGGAGGGAGATGTG
TCTACCCTGGAGCAGGGAGAGGGCAGGCTGCAGCTGCGGGCCTCGGTGAAGAGAAAAAGC
AGGCCCTGCTCCTTGCCTGTGTCCGAGCTGGAGACGGTGATCGCGTCAGCCTGCGGGGAC
CCCGAGACCCCGCGGACACACTACATCCGCATCCACACCCTGCTGCACAGCATGCCCTCC
GCCCAGGGCGGCAGCGCGGCAGAGGAGGAGGACGGCGCGGAGGAGGAGTCCACCCTCAAG
GACTCCTCGGAGAAGGATGGGCTCAGCGAGGTGGACACGGTGGCCGCTGACCCGTCTGCC
CTGGAAGAGGACAGAGAAGAGCCCGAGGGGGCTACTCCAGGCACGGCGCACCCTGGCCAC
TCCGGGGGCCACTTCCCCAGCCTGGCCAATGGCGCGGCCCAGGATGGCGACACGCACCCC
AGCACCGGGAGCGAGAGCGACTCCAGCCCCAGGCAAGGCGGGGACCACAGTTGCGAGGGC
TGTGACGCGTCCTGCTGCAGCCCCTCGTGCTACAGCTCCTCGTGCTACAGCACGTCCTGC
TACAGCAGCTCGTGCTACAGCGCCTCGTGCTACAGCCCCTCCTGCTACAACGGCAACAGG
TTCGCCAGCCACACGCGCTTCTCCTCCGTGGACAGCGCCAAGATCTCCGAGAGCACGGTC
TTCTCCTCGCAAGACGACGAGGAGGAGGAGAACAGCGCGTTCGAGTCGGTACCCGACTCC
ATGCAGAGCCCTGAGCTGGACCCGGAGTCCACGAACGGCGCTGGGCCGTGGCAAGACGAG
CTGGCCGCCCCTAGCGGGCACGTGGAAAGAAGCCCGGAAGGTCTGGAATCCCCCGTGGCA
GGTCCAAGCAATCGGAGAGAAGACTGGGAAGCTCGAATTGACAGCCACGGGCGGGTCTTT
TATGTGGACCACGTGAACCGCACAACCACCTGGCAGCGTCCGACGGCAGCAGCCACCCCG
GATGGCATGCGGAGATCGGGGTCCATCCAGCAGATGGAGCAACTCAACAGGCGGTATCAA
AACATTCAGCGAACCATTGCAACAGAGAGGTCCGAAGAAGATTCTGGCAGCCAAAGCTGC
GAGCAAGCCCCAGCAGGAGGAGGCGGAGGTGGAGGGAGTGACTCAGAAGCCGAATCTTCC
CAGTCCAGCTTAGATCTAAGGAGAGAGGGGTCACTTTCTCCAGTGAACTCACAAAAAATC
ACCTTGCTGCTGCAGTCCCCAGCGGTCAAGTTCATCACCAACCCCGAGTTCTTCACTGTG
CTACACGCCAATTATAGTGCCTACCGAGTCTTCACCAGTAGCACCTGCTTAAAGCACATG
ATTCTGAAAGTCCGACGGGATGCTCGCAATTTTGAACGCTACCAGCACAACCGGGACTTG
GTGAATTTCATCAACATGTTCGCAGACACTCGGCTGGAACTGCCCCGGGGCTGGGAGATC
AAAACGGACCAGCAGGGAAAGTCTTTTTTCGTGGACCACAACAGTCGAGCTACCACTTTC
ATTGACCCCCGAATCCCTCTTCAGAACGGTCGTCTTCCCAATCATCTAACTCACCGACAG
CACCTCCAGAGGCTCCGAAGTTACAGCGCCGGAGAGGCCTCAGAAGTTTCTAGAAACAGA
GGAGCCTCTTTACTGGCCAGGCCAGGACACAGCTTAGTAGCTGCTATTCGAAGCCAACAT
CAACATGAGTCATTGCCACTGGCATATAATGACAAGATTGTGGCATTTCTTCGCCAGCCA
AACATTTTTGAAATGCTGCAAGAGCGTCAGCCAAGCTTAGCAAGAAACCACACACTCAGG
GAGAAAATCCATTACATTCGGACTGAGGGTAATCACGGGCTTGAGAAGTTGTCCTGTGAT
GCGGATCTGGTCATTTTGCTGAGTCTCTTTGAAGAAGAGATTATGTCCTACGTCCCCCTG
CAGGCTGCCTTCCACCCTGGGTATAGCTTCTCTCCCCGATGTTCACCCTGTTCTTCACCT
CAGAACTCCCCAGGTTTACAGAGAGCCAGTGCAAGAGCCCCTTCCCCCTACCGAAGAGAC
TTTGAGGCCAAGCTCCGCAATTTCTACAGAAAACTGGAAGCCAAAGGATTTGGTCAGGGT
CCGGGGAAAATTAAGCTCATTATTCGCCGGGATCATTTGTTGGAGGGAACCTTCAATCAG
GTGATGGCCTATTCGCGGAAAGAGCTCCAGCGAAACAAGCTCTACGTCACCTTTGTTGGA
GAGGAGGGCCTGGACTACAGTGGCCCCTCGCGGGAGTTCTTCTTCCTTCTGTCTCAGGAG
CTCTTCAACCCTTACTATGGACTCTTTGAGTACTCGGCAAATGATACTTACACGGTGCAG
ATCAGCCCCATGTCCGCATTTGTAGAAAACCATCTTGAGTGGTTCAGGTTTAGCGGTCGC
ATCCTGGGTCTGGCTCTGATCCATCAGTACCTTCTTGACGCTTTCTTCACGAGGCCCTTC
TACAAGGCACTCCTGAGACTGCCCTGTGATTTGAGTGACCTGGAATATTTGGATGAGGAA
TTCCACCAGAGTTTGCAGTGGATGAAGGACAACAACATCACAGACATCTTAGACCTCACT
TTCACTGTTAATGAAGAGGTTTTTGGACAGGTCACGGAAAGGGAGTTGAAGTCTGGAGGA
GCCAACACACAGGTGACGGAGAAAAACAAGAAGGAGTACATCGAGCGCATGGTGAAGTGG
CGGGTGGAGCGCGGCGTGGTACAGCAGACCGAGGCGCTGGTGCGCGGCTTCTACGAGGTT
GTAGACTCGAGGCTGGTGTCCGTGTTTGATGCCAGGGAGCTGGAGCTGGTGATAGCTGGC
ACCGCGGAAATCGACCTAAATGACTGGCGGAATAACACTGAGTACCGGGGAGGTTACCAC
GATGGGCATCTTGTGATCCGCTGGTTCTGGGCTGCGGTGGAGCGCTTCAATAATGAGCAG
AGGCTGAGATTACTGCAGTTTGTCACGGGAACATCCAGCGTGCCCTACGAAGGCTTCGCA
GCCCTCCGTGGGAGCAATGGGCTTCGGCGCTTCTGCATAGAGAAATGGGGGAAAATTACT
TCTCTCCCCAGGGCACACACATGCTTCAACCGACTGGATCTTCCACCGTATCCCTCGTAC
TCCATGTTGTATGAAAAGCTGTTAACAGCAGTAGAGGAAACCAGCACCTTTGGACTTGAG
TGAGGACATGGAACCTCGCCTGACATTTTCCTGGCCAGTGACATCACCCTTCCTGGGATG
ATCCCCTTTTCCCTTTCCCTTAATCAACTCTCCTTTGATTTTGGTATTCCATGATTTTTA
TTTTCAAAC
Human NEDL2 nucleic acid sequence (uniprot.org/uniprot/Q9P2P5).
(SEQ ID NO: 56)
AGAGTTCCATCAGAGCCTGCAGTGGATGAAAGACAATGATATCCATGACATCCTAGACCT
CACGTTCACTGTGAACGAAGAAGTATTTGGGCAGATAACTGAACGAGAATTAAAGCCAGG
GGGTGCCAATATCCCAGTTACAGAGAAGAACAAGAAGGAGTACATCGAGAGGATGGTGAA
GTGGAGGATTGAGAGGGGTGTTGTACAGCAAACAGAGAGCTTAGTGCGTGGCTTCTATGA
GGTGGTGGATGCCAGGCTGGTATCTGTTTTTGATGCAAGAGAACTGGAATTGGTCATCGC
AGGCACAGCTGAAATAGACCTAAGTGATTGGAGAAACAACACAGAATATAGAGGAGGATA
CCATGACAATCATATTGTAATTCGGTGGTTCTGGGCTGCAGTGGAAAGATTCAACAATGA
ACAACGACTAAGGTTGTTACAGTTTGTTACAGGCACATCCAGCATTCCCTATGAAGGATT
TGCTTCACTCCGAGGGAGTAACGGCCCAAGAAGATTCTGTGTGGAGAAATGGGGGAAAAT
CACTGCTCTTCCCAGAGCGCATACATGTTTTAACCGTCTGGATCTGCCTCCCTACCCATC
CTTTTCCATGCTTTATGAAAAACTGTTGACAGCAGTTGAAGAAACCAGTACTTTTGGACT
TGAGTGACCTGGAAGCTGAATGCCCATCTCTGTGGACAGGCAGTTTCAGAAGCTGCCTTC
TAGAAGAATGATTGAACATTGGAAGTTTCAAGAGGATGCTTCCTTTAGGATAAAGCTACG
TGCTGTTGTTTTCCAGGAACAAGTGCTCTGTCACATTTGGGGACTGGAGATGAGTCCTCT
TGGAAGGATTTGGGTGAGCTTGATGCCCAGGGAACAACCCAACCGTCTTTCAATCAACAG
TTCTTGACTGCCAAACTTTTTCCATTTGTTATGTTCCAAGACAAAGATGAACCCATACAT
GATCAGCTCCACGGTAATTTTTAGGGACTCAGGAGAATCTTGAAACTTACCCTTGAACGT
GGTTCAAGCCAAACTGGCAGCATTTGGCCCAATCTCCAAATTAGAGCAAGTTAAATAATA
TAATAAAAGTAAATATATTTCCTGAAAGTACATTCATTTAAGCCCTAAGTTATAACAGAA
TATTCATTTCTTGCTTATGAGTGCCTGCATGGTGTGCACCATAGGTTTCCGCTTTCATGG
GACATGAGTGAAAATGAAACCAAGTCAATATGAGGTACCTTTACAGATTTGCAATAAGAT
GGTCTGTGACAATGTATATGCAAGTGGTATGTGTGTAATTATGGCTAAAGACAAACCATT
ATTCAGTGAATTACTAATGACAGATTTTATGCTTTATAATGCATGAAAACAATTTTAAAA
TAACTAGCAATTAATCACAGCATATCAGGAAAAAGTACACAGTGAGTTCTGTTTATTTTT
TGTAGGCTCATTATGTTTATGTTCTTTAAGATGTATATAAGAACCTACTTATCATGCTGT
ATGTATCACTCATTCCATTTTCATGTTCCATGCATACTCGGGCATCATGCTAATATGTAT
CCTTTTAAGCACTCTCAAGGAAACAAAAGGGCCTTTTATTTTTATAAAGGTAAAAAAAAT
TCCCCAAATATTTTGCACTGAATGTACCAAAGGTGAAGGGACATTACAATATGACTAACA
GCAACTCCATCACTTGAGAAGTATAATAGAAAATAGCTTCTAAATCAAACTTCCTTCACA
GTGCCGTGTCTACCACTACAAGGACTGTGCATCTAAGTAATAATTTTTTAAGATTCACTA
TATGTGATAGTATGATATGCATTTATTTAAAATGCATTAGACTCTCTTCCATCCATCAAA
TACTTTACAGGATGGCATTTAATACAGATATTTCGTATTTCCCCCACTGCTTTTTATTTG
TACAGCATCATTAAACACTAAGCTCAGTTAAGGAGCCATCAGCAACACTGAAGAGATCAG
TAGTAAGAATTCCATTTTCCCTCATCAGTGAAGACACCACAAATTGAAACTCAGAACTAT
ATTTCTAAGCCTGCATTTTCACTGATGCATAATTTTCTTATTAATATTAAGAGACAGTTT
TTCTATGGCATCTCCAAAACTGCATGACATCACTAGTCTTACTTCTGCTTAATTTTATGA
GAAGGTATTCTTCATTTTAATTGCTTTTGGGATTACTCCACATCTTTGTTTATTTCTTGA
CTAATCAGATTTTCAATAGAGTGAAGTTAAATTGGGGGTCATAAAAGCATTGGATTGACA
TATGGTTTGCCAGCCTATGGGTTTACAGGCATTGCCCAAACATTTCTTTGAGATCTATAT
TTATAAGCAGCCATGGAATTCCTATTATGGGATGTTGGCAATCTTACATTTTATAGAGGT
CATATGCATAGTTTTCATAGGTGTTTTGTAAGAACTGATTGCTCTCCTGTGAGTTAAGCT
ATGTTTACTACTGGGACCCTCAAGAGGAATACCACTTATGTTACACTCCTGCACTAAAGG
CACGTACTGCAGTGTGAAGAAATGTTCTGAAAAAGGGTTATAGAAATCTGGAAATAAGAA
AGGAAGAGCTCTCTGTATTCTATAATTGGAAGAGAAAAAAAGAAAAACTTTTAACTGGAA
ATGTTAGTTTGTACTTATTGATCATGAATACAAGTATATATTTAATTTTGCAAAAAAAAA
AAAAAAAAAAAAAAG

Some aspects of this invention provide expression constructs that encode any of the proteins, nucleic acids, such as RNAs, or fusions thereof described herein.

Nucleic acids encoding any of the proteins and/or nucleic acid (including RNA) described herein, may be in any number of nucleic acid vectors known in the art. Vectors suitable for use in the compositions and methods of the present invention include both viral and nonviral products and additional means for introducing nucleic acid(s) into cells.

Expression of any of the proteins and/or nucleic acid (including RNA) described herein, may be controlled by any regulatory sequence (e.g., a promoter sequence) known in the art. Regulatory sequences, as described herein, are nucleic acid sequences that regulate the expression of a nucleic acid sequence. A regulatory or control sequence may include sequences that are responsible for expressing a particular nucleic acid or may include other sequences, such as heterologous, synthetic, or partially synthetic sequences. The sequences can be of eukaryotic, prokaryotic, or viral origin that stimulate or repress transcription of a gene in a specific or non-specific manner and in an inducible or non-inducible manner. Regulatory or control regions may include origins of replication, RNA splice sites, introns, chimeric or hybrid introns, promoters, enhancers, transcriptional termination sequences, poly A sites, locus control regions, signal sequences that direct the polypeptide into the secretory pathways of the target cell. A heterologous regulatory region is a regulatory region not naturally associated with the expressed nucleic acid it is linked to. Included among the heterologous regulatory regions are regulatory regions from a different species, regulatory regions from a different gene, hybrid regulatory sequences, and regulatory sequences that do not occur in nature, but which are designed by one of ordinary skill in the art.

### Exemplary Cells Producing ARMMs Containing Payload

A microvesicle-producing cell of the present invention may be a cell containing any of the expression constructs, any of the fusion proteins, or any of the payloads of molecules (e.g., biological molecules, small molecules, proteins, and nucleic acids described herein. For example, an inventive microvesicle-producing cell may contain one or more recombinant expression constructs encoding (1) an ARRDC1 protein, or PSAP (SEQ ID NO: 1) motif-containing variant thereof and (2) an RNA binding protein (e.g., a Tat protein), that is associated with the ARRDC1 protein, or PSAP (SEQ ID NO: 1) motif-containing variant thereof. In some embodiments, a microvesicle-producing cell may contain one or more recombinant expression constructs encoding (1) an ARRDC1 protein, or PSAP (SEQ ID NO: 1) motif-containing variant thereof, and (2) a payload protein, such as an RNA binding protein fused to at least one WW domain, or variant thereof, under the control of a heterologous promoter. In certain embodiments, the expression construct in the microvesicle producing cell encodes a payload protein with one or more WW domains or variants thereof. In certain embodiments, an expression construct in the microvesicle producing cell encodes an RNA that associates with (e.g., binds specifically) an RNA binding protein, for example a therapeutic RNA.

Any of the expression constructs, described herein, may be stably inserted into the genome of the cell. In some embodiments, the expression construct is maintained in the cell, but not inserted into the genome of the cell. In some embodiments, the expression construct is in a vector, for example, a plasmid vector, a cosmid vector, a viral vector, or an artificial chromosome. In some embodiments, the expression construct further comprises additional sequences or elements that facilitate the maintenance and/or the replication of the expression construct in the microvesicle-producing cell, or that improve the expression of the fusion protein in the cell. Such additional sequences or elements may include, for example, an origin of replication, an antibiotic resistance cassette, a polyA sequence, and/or a transcriptional isolator. Some expression constructs suitable for the generation of microvesicle producing cells according to aspects of this invention are described elsewhere herein. Methods and reagents for the generation of additional expression constructs suitable for the generation of microvesicle producing cells according to aspects of this invention will be apparent to those of skill in the art based on the present disclosure. In some embodiments, the microvesicle producing cell is a mammalian cell, for example, a mouse cell, a rat cell, a hamster cell, a rodent cell, or a nonhuman primate cell. In some embodiments, the microvesicle producing cell is a human cell.

One skilled in the art may employ conventional techniques, such as molecular or cell biology, virology, microbiology, and recombinant DNA techniques. Exemplary techniques are explained fully in the literature. For example, one may rely on the following general texts to make and use the invention: Sambrook et al., Molecular Cloning: A Laboratory Manual, Second Edition (1989) Cold Spring Harbor Laboratory Press, Cold Spring Harbor, New York, and Sambrook et al., Third Edition (2001); DNA Cloning: A Practical Approach, Volumes I and II (D. N. Glover ed. 1985); Oligonucleotide Synthesis (M. J. Gaited. 1984); Nucleic Acid Hybridization (B. D. Hames & S. J. Higgins eds. (1985)); Transcription and Translation Hames & Higgins, eds. (1984); Animal Cell Culture (RI. Freshney, ed. (1986)); Immobilized Cells And Enzymes (IRL Press, (1986)): Gennaro et al., (eds.) Remington's Pharmaceutical Sciences, 18th edition: B. Perbal, A Practical Guide To Molecular Cloning (1984); F. M. Ausubel et al., (eds.), Current Protocols in Molecular Biology, John Wiley & Sons, Inc. (updates through 2001), Coligan et al., (eds.), Current Protocols in Immunology, John Wiley & Sons, Inc. (updates through 2001); W. Paul et al., (eds.) Fundamental Immunology, Raven Press; E. J. Murray et al., (ed.) Methods in Molecular Biology: Gene Transfer and Expression Protocols, The Humana Press Inc. (1991)(especially vol. 7); and J. E. Celis et al., Cell Biology: A Laboratory Handbook, Academic Press (1994).

### Delivery of ARMMs Containing Payload Molecules

The inventive microvesicles (e.g., ARMMs containing any of the expression constructs and/or any of the payload of molecules (e.g., therapeutic agents, biological molecules, small molecules, proteins, and nucleic acids (e.g., DNA, RNA), DNA plasmids, siRNA, shRNA, mRNA)), may optionally further comprise a targeting moiety. The targeting moiety may be used to target the delivery of ARMMs to specific cell types, resulting in the release of the contents of the ARMM into the cytoplasm of the specific targeted cell type. A targeting moiety may be a viral envelope protein, or portion thereof, that normally functions to aid viral attachment and entry into cells. The viral envelope protein may allow for the targeting of cells of the CNS. Viral envelope proteins include, but are not limited to, vesicular stomatitis virus G protein (VSV-G: Genbank Accession and Version Number: AJ318514.1) or rabies virus glycoprotein (RVG; Genbank Accession and Version Number: M38452.1). VSV-G protein, facilitates viral entry by mediating viral attachment to an LDL receptor (LDLR) or an LDLR family member present on a target cell. Subsequent to binding, the VSV-G-LDLR complex is rapidly endocytosed and proceeds to mediate the fusion of the viral envelope with the endosomal membrane. VSV-G enters the cell through partially clathrin-coated vesicles; virus-containing vesicles contain more clathrin and clathrin adaptor than conventional vesicles. VSV-G is a common coat protein for vector expression systems used to introduce genetic material into in vitro systems or animal models, mainly because of its extremely broad tropism. RVG is a trimeric and surface-exposed viral coat protein known to use the nicotinic acetylcholine receptor and the low affinity nerve growth factor receptor for viral entry. In some embodiments, a viral envelope protein (e.g., VSV-G, RVG) facilitates binding (e.g., targeting) of the ARMMs to targeted cells.

A targeting moiety may selectively bind a surface antigen of the target cell. For example, the targeting moiety may be a membrane-bound immunoglobulin, an integrin, a receptor, a receptor ligand, an aptamer, a small molecule, or a variant thereof. Any number of cell surface proteins may also be included in an ARMM to facilitate the binding of an ARMM to a target cell and/or to facilitate the uptake of an ARMM into a target cell. Integrins, receptor tyrosine kinases, G-protein coupled receptors, and membrane-bound immunoglobulins suitable for use with embodiments of this invention will be apparent to those of skill in the art and the invention is not limited in this respect. For example, in some embodiments, the integrin is an α1β1, α2β1, α4β1, α5β1, α6β1, α1β2, αMβ2, αIIbβ3, αVβ3, αVβ5, αVβ6, or a α6β4 integrin. In some embodiments, the receptor tyrosine kinase is a an EGF receptor (ErbB family), insulin receptor, PDGF receptor, FGF receptor. VEGF receptor, HGF receptor, Trk receptor, Eph receptor, AXL receptor, LTK receptor, TIE receptor, ROR receptor, DDR receptor, RET receptor, KLG receptor, RYK receptor, or MuSK receptor. In some embodiments, the G-protein coupled receptor is a rhodopsin-like receptor, the secretin receptor, metabotropic glutamate/pheromone receptor, cyclic AMP receptor, frizzled/smoothened receptor, CXCR4, CCR5, or beta-adrenergic receptor.

Additional molecules, such as synthetic small molecules or natural products, can be modified to associate with an ARMM protein (e.g., TSG101 or ARRDC1) for the purpose of targeting. This association can facilitate their incorporation with ARMMs, which in turn can be used to augment delivery of ARMMs to target cells. The incorporation of a cleavable linker may be used to allow the small molecule to be released upon delivery in or to a target cell. As a non-limiting example, a small molecule can be linked to biotin, thereby allowing it to associate with an ARRDC1 protein which is fused to a streptavidin. As another non-limiting example, a small molecule can be linked to synthetic high affinity ligand that specifically binds to a mutant form of FKBP12 such as FKBP12(F36V) (Yang, W., et al., “Investigating protein-ligand interactions with a mutant FKBP possessing a designed specificity pocket” J. Med. Chem., 43(6):1135-1142 (2000)), which will associate with an ARRDC1 protein which is fused to FKBP12(F36V). The association of the small molecule to an ARMM protein (e.g., TSG101 or ARRDC1), facilitates the loading of the small molecule into the ARRDC1-containing ARMM.

Some aspects of this invention relate to the recognition that ARMMs are taken up by target cells (e.g., cells of the retina, including, retinal pigmented epithelium cells, photoreceptor cells, horizontal cells, bipolar cells, amacrine cells, and/or ganglion cells) and ARMM uptake results in the release of the contents of the ARMM into the cytoplasm of the target cells. In some embodiments, the payload is an agent that affects a desired change in the target cell, for example, a change in cell survival, proliferation rate, a change in differentiation stage, a change in cell identity, a change in chromatin state, a change in the transcription rate of one or more genes, a change in the transcriptional profile, or a post-transcriptional change in gene compression of the target cell, and the like. It will be understood by those of skill in the art, that the agent to be delivered will be chosen according to the desired effect in the target cell (e.g., a base editor).

In some embodiments, cells from a subject are obtained and a payload is delivered to the cells by a system or method provided herein ex vivo. In some embodiments, the treated cells are selected for those cells in which a desired gene is expressed or repressed. In some embodiments, treated cells carrying a desired payload are returned to the subject they were obtained from.

In some embodiments, the ARMMs further comprise a detectable label. In certain embodiments, detectably labeled ARMMs allow for the labeling of target cells without genetic manipulation. Detectable labels suitable for direct delivery to target cells are known in the art, and include, but are not limited to, fluorescent proteins, fluorescent dyes, membrane-bound dyes, and enzymes, for example, membrane-bound or cytosolic enzymes, catalyzing the reaction resulting in a detectable reaction product. Detectable labels suitable according to some aspects of this invention further include membrane-bound antigens, for example, membrane-bound ligands that can be detected with commonly available antibodies or antigen binding agents. Detectably labeled ARRMs find use in various diagnostic and analytical methods and applications.

In some embodiments, ARMMs are provided that comprise a payload RNA that encodes a transcription factor, a transcriptional repressor, a fluorescent protein, a kinase, a phosphatase, a protease, a ligase, a chromatin modulator, a recombinase, and the like. In some embodiments, ARMMs are provided that comprise a payload RNA that inhibits the expression of a transcription factor, a transcriptional repressor, a fluorescent protein, a kinase, a phosphatase, a protease, a ligase, a chromatin modulator, or a recombinase. In some embodiments, the payload RNA is a therapeutic RNA. In some embodiments, the payload RNA is an RNA that affects a change in the state or identity of a target cell. For example, in some embodiments, the payload RNA encodes a reprogramming factor. Suitable transcription factors, transcriptional repressors, fluorescent proteins, kinases, phosphatases, proteases, ligases, chromatin modulators, recombinases, and reprogramming factors may be encoded by a payload RNA that is associated with a binding RNA to facilitate their incorporation into ARMMs and their function may be tested by any methods that are known to those skilled in the art, and the invention is not limited in this respect.

Methods for isolating the ARMMs described herein are also provided. One exemplary method includes employing conventional techniques of collecting culture medium, or supernatant, from a cell culture comprising microvesicle-producing cells. In some embodiments, the cell culture comprises cells obtained from a subject, for example, cells suspected to exhibit a pathological phenotype, for example, a hyperproliferative phenotype. In some embodiments, the cell culture comprises genetically engineered cells producing ARMMs, for example, cells expressing a recombinant protein, for example, a recombinant ARRDC1 or TSG101 protein, such as an ARRDC1 or TSG101 protein, optionally fused to an RNA binding protein (e.g., a Tat protein) or variant thereof. In some embodiments, the supernatant is pre-cleared of cellular debris by centrifugation, for example, by two consecutive centrifugations of increasing G value (e.g., 500 G and 2000 G). In some embodiments, the method comprises passing the supernatant through a 0.2 μm filter, eliminating all large pieces of cell debris and whole cells. In some embodiments, the supernatant is subjected to ultracentrifugation, for example, at 120,000 G for 2 hours, depending on the volume of centrifugate. The pellet obtained comprises microvesicles. In some embodiments, exosomes are depleted from the microvesicle pellet by staining and/or sorting (e.g., by FACS or MACS) using an exosome marker as described herein. Isolated or enriched ARMMs can be suspended in culture media or a suitable buffer, as described herein.

### Methods of ARMMs-Mediated Delivery of Payload to Cells

Some aspects of this invention provide a method of delivering an agent (e.g., a therapeutic agent or agents) to a target cell of the eye. The target cell can be contacted with an ARMM in different ways. For example, a target cell may be contacted directly with an ARMM as described herein, or with an isolated ARMM from a microvesicle producing cell. The contacting can be done in vitro by administering the ARMM to the target cell in a culture dish, or in vivo by administering the ARMM to a subject. In some embodiments, the ARMMs are produced from cells obtained from a subject. In some embodiments, ARMMs that are produced from a cell obtained from a particular subject are administered to the same subject. Conversely, in some other embodiments, ARMMs produced from a cell that was obtained from the subject are administered to a different subject. As one example, a cell may be obtained from a subject and engineered to express one or more of the constructs provided herein (e.g., engineered to express a payload RNA associated with a binding RNA, an ARRDC1 protein, an ARRDC1 protein fused to an RNA binding protein, an RNA binding protein fused to a WW domain, a Cas9 protein, a base editor, guide and regulator sequences, and the like).

Alternatively, a target cell can be contacted with a microvesicle producing cell as described herein, for example, contacted in vitro by co-culturing the target cell and the microvesicle producing cell, or contacted in vivo by administering a microvesicle producing cell to a subject harboring the target cell. Accordingly, the method may include contacting the target cell with a microvesicle, for example, an ARMM containing any of the payloads to be delivered, as described herein. The target cell may be contacted with a microvesicle-producing cell, as described herein, or with an isolated microvesicle that has a lipid bilayer, an ARRDC1 protein or variant thereof, a payload (e.g., therapeutic agent(s)), and optionally a viral envelope protein.

It should be appreciated that the target cell may be of any origin, for example, from an organism. In some embodiments, the target cell is a mammalian cell. Some non-limiting examples of a mammalian cell include, without limitation, a mouse cell, a rat cell, a hamster cell, a rodent cell, and a nonhuman primate cell. In some embodiments, the target cell is a human cell. It should also be appreciated that the target cell may be of any cell type, and preferentially, cells of the eye. In other cases, the target cell may be any differentiated cell type found in a subject. In some embodiments, the target cell is a cell in vitro, and the method includes administering the microvesicle to the cell in vitro, or co-culturing the target cell with the microvesicle-producing cell in vitro. In some embodiments, the target cell is a cell in a subject, and the method comprises administering the microvesicle or the microvesicle-producing cell to the subject. In some embodiments, the subject is a mammalian subject, for example, a rodent, a mouse, a rat, a hamster, or a non-human primate. In some embodiments, the subject is a human subject.

In some embodiments, the target cell is a pathological cell. In some embodiments, the target cell is a cancer cell. In some embodiments, the microvesicle is associated with a binding agent that selectively binds an antigen on the surface of the target cell. In some embodiments, the compositions and methods of the present invention comprise one or more targeting ligands that associate (e.g., bind) to one or more targeting receptors. In some embodiments, the antigen of the target cell is a cell surface antigen. In some embodiments, the binding agent is a membrane-bound immunoglobulin, an integrin, a receptor, a receptor ligand, or a lectin, among other suitable candidate molecules and moieties. Suitable surface antigens of target cells (e.g., cells of the eye) are known to those of skill in the art as are suitable binding agents that specifically bind such antigens. Methods for producing membrane-bound binding agents, for example, membrane-bound immunoglobulins, membrane-bound antibodies, or antibody fragments that specifically bind a surface antigen expressed on the surface of cells, are also known to those of skill in the art. The choice of the binding agent will depend, of course, on the identity or the type of target cell. Cell surface antigens specifically expressed on various types of optical tissues and cells that can be targeted by ARMMs comprising membrane-bound binding agents will be apparent to those of skill in the art. It will be appreciated that the present invention is not limited in this respect.

### EXAMPLES

### Example 1 ARMMs Production

Expi293™ cells (Thermo Fisher Scientific, Waltham, MA) were seeded in Expi293™ expression medium (Thermo Fisher Scientific) at a density of 36/ml in 10 ml. For each 10 ml of cells, 5 μg of ARRDC1-Cre plasmid (SEQ ID NO: 48) and 5 μg of VSVG plasmid (SEQ ID NO: 49), both below, were mixed with 600p1 OptiMEM™ (Thermo Fisher Scientific) for less than five (5) minutes. 33 μl of expifectamine was mixed with 567 μl of OptiMEM™ and then mixed with DNA and OptiMEM™ mixture. Samples were left at room temperature for 10 minutes and added to Expi™ cells for about 16-18 hours. After the incubation the media was changed the following morning and cells were centrifuged at 500×g for five (5) minutes and reseeded in a final volume of 40 ml medium. Cells were incubated for an additional 48 hours.

After the 48 hour incubation, cells were placed in a 50 ml centrifuge tubes and centrifuged for five (5) minutes at 500×g. The supernatant was removed and centrifuged again at 2000×g for 10 minutes. The supernatant was put through 0.22 μM vacuum filter. The supernatant was placed in 38.5 ml ultracentrifuge tubes (Beckman-Coulter Life Sciences, Brea, CA) and spun at 174,900×g for two (2) hours at 4° C. The supernatant was removed, and the pellet was resuspended in PBS and quantified by nanoparticle tracking analysis.

ARRDC1-Cre Sequence
(SEQ ID NO: 48)
atggggcgagtgcagctcttcgagatcagcctgagccacggccgcgtcgtctacagccccgg
ggagccgttggctgggaccgtgcgcgtgcgcctgggggcaccgctgccgttccgagccatcc
gggtgacctgcataggttcctgcggggtctccaacaaggctaatgacacagcgtgggtagtg
gaggagggttacttcaacagttccctgtcgctggcagacaaggggagcctgcccgctggaga
gcacagcttccccttccagttcctgcttcctgccactgcacccacgtcctttgagggtcctt
tcgggaagatcgtgcaccaggtgagggccgccatccacacgccacggttttccaaggatcac
aagtgcagcctcgtgttctatatcttgagccccttgaacctgaacagcatcccagacattga
gcaacccaacgtggcctctgccaccaagaagttctcctacaagctggtgaagacgggcagcg
tggtcctcacagccagcactgatctccgcggctatgtggtggggcaggcactgcagctgcat
gccgacgttgagaaccagtcaggcaaggacaccagccctgtggtggccagtctgctgcagaa
agtgtcctataaggccaagcgctggatccacgacgtacggaccattgcggaggtggagggtg
cgggcgtcaaggcctggcggcgggcgcagtggcacgagcagatcctggtgcctgccttgccc
cagtcggccctgccgggctgcagcctcatccacatcgactactacttacaggtctctctgaa
ggcgccggaagctactgtgaccctcccggtcttcattggcaatattgctgtgaaccatgccc
cagtgagcccccggccaggcctggggctgcctcctggggccccacccctggtggtgccttcc
gcaccaccccaggaggaggctgaggctgaggctgcggctggcggcccccacttcttggaccc
cgtcttcctctccaccaagagccattcgcagcggcagcccctgctggccaccttgagttctg
tgcctggtgcgccggagccctgccctcaggatggcagccctgcctcacacccgctgcaccct
cccttgtgcatttcaacaggtgccactgtcccctactttgcagagggctccggggggccagt
gcccactaccagcaccttgattcttcctccagagtacagttcttggggctacccctatgagg
ccccaccgtcttatgagcagagctgcggcggcgtggaacccagcctgacccctgagagctct
ggtggcggaggctcgggcggaggtgggtcgggtggcggcggatcaatgggcccaaagaagaa
gagaaaggtttcgaatttactgaccgtacaccaaaatttgcctgcattaccggtcgatgcaa
cgagtgatgaggttcgcaagaacctgatggacatgttcagggatcgccaggcgttttctgag
catacctggaaaatgcttctgtccgtttgccggtcgtgggcggcatggtgcaagttgaataa
ccggaaatggtttcccgcagaacctgaagatgttcgcgattatcttctatatcttcaggcgc
gcggtctggcagtaaaaactatccagcaacatttgggccagctaaacatgcttcatcgtcgg
tccgggctgccacgaccaagtgacagcaatgctgtttcactggttatgcggcggatccgaaa
agaaaacgttgatgccggtgaacgtgcaaaacaggctctagcgttcgaacgcactgatttcg
accaggttcgttcactcatggaaaatagcgatcgctgccaggatatacgtaatctggcattt
ctggggattgcttataacaccctgttacgtatagccgaaattgccaggatcagggttaaaga
tatctcacgtactgacggtgggagaatgttaatccatattggcagaacgaaaacgctggtta
gcaccgcaggtgtagagaaggcacttagcctgggggtaactaaactggtcgagcgatggatt
tccgtctctggtgtagctgatgatccgaataactacctgttttgccgggtcagaaaaaatgg
tgttgccgcgccatctgccaccagccagctatcaactcgcgccctggaagggatttttgaag
caactcatcgattgatttacggcgctaaggatgactctggtcagagatacctggcctggtct
ggacacagtgcccgtgtcggagccgcgcgagatatggcccgcgctggagtttcaataccgga
gatcatgcaagctggtggctggaccaatgtaaatattgtcatgaactatatccgtaacctgg
atagtgaaacaggggcaatggtgcgcctgctggaagatggcgattag
VSVG Sequence
(SEQ ID NO: 49)
atgaagtgccttttgtacttagcctttttattcattggggtgaattgcaagttcaccatagt
ttttccacacaaccaaaaaggaaactggaaaaatgttccttctaattaccattattgcccgt
caagctcagatttaaattggcataatgacttaataggcacagccttacaagtcaaaatgccc
aagagtcacaaggctattcaagcagacggttggatgtgtcatgcttccaaatgggtcactac
ttgtgatttccgctggtatggaccgaagtatataacacattccatccgatccttcactccat
ctgtagaacaatgcaaggaaagcattgaacaaacgaaacaaggaacttggctgaatccaggc
ttccctcctcaaagttgtggatatgcaactgtgacggatgccgaagcagtgattgtccaggt
gactcctcaccatgtgctggttgatgaatacacaggagaatgggttgattcacagttcatca
acggaaaatgcagcaattacatatgccccactgtccataactctacaacctggcattctgac
tataaggtcaaagggctatgtgattctaacctcatttccatggacatcaccttcttctcaga
ggacggagagctatcatccctgggaaaggagggcacagggttcagaagtaactactttgctt
atgaaactggaggcaaggcctgcaaaatgcaatactgcaagcattggggagtcagactccca
tcaggtgtctggttcgagatggctgataaggatctctttgctgcagccagattccctgaatg
cccagaagggtcaagtatctctgctccatctcagacctcagtggatgtaagtctaattcagg
acgttgagaggatcttggattattccctctgccaagaaacctggagcaaaatcagagcgggt
cttccaatctctccagtggatctcagctatcttgctcctaaaaacccaggaaccggtcctgc
tttcaccataatcaatggtaccctaaaatactttgagaccagatacatcagagtcgatattg
ctgctccaatcctctcaagaatggtcggaatgatcagtggaactaccacagaaagggaactg
tgggatgactgggcaccatatgaagacgtggaaattggacccaatggagttctgaggaccag
ttcaggatataagtttcctttatacatgattggacatggtatgttggactccgatcttcatc
ttagctcaaaggctcaggtgttcgaacatcctcacattcaagacgctgcttcgcaacttcct
gatgatgagagtttattttttggtgatactgggctatccaaaaatccaatcgagcttgtaga
aggttggttcagtagttggaaaagctctattgcctcttttttctttatcatagggttaatca
ttggactattcttggttctccgagttggtatccatctttgcattaaattaaagcacaccaag
aaaagacagatttatacagacatagagatgaaccgacttggaaagtaa

### Nanoparticle Tracking Analysis

ARMMs were analyzed and quantitated by the ZetaView® instrument (Particle Metrix GmbH, Inning am Ammersee, Germany). Samples containing vesicles were diluted with phosphate-buffered saline (PBS). The samples were subject to nanoparticle tracking analysis after dilution.

### Example 2

### Evaluation in Subretinal Injection of ARMMs into Göttingen Minipigs

Briefly, ocular biodistribution of ARMMs was evaluated in a Göttingen minipig model via subretinal delivery. Adult Göttingen minipigs were subretinally injected with ARMMs that were loaded with green fluorescent protein (ARRDC1-GFP-ARMMs). (SEQ ID NO: 50) and SEQ ID NO: 51). The dose was extrapolated from previously conducted mouse subretinal studies and was equivalent to a potential human dose, given the comparable size, physiology, and anatomy of pig and human eyes. For histological and immunohistochemical assessment, a time-course analysis was conducted by harvesting the eyes at 6 hours, 12 hours, and 24 hours after subretinal injection of ARMMs. GFP staining was used as a proxy for ARMMs uptake. Co-staining of GFP with the following cell type-specific markers were examined: RPE65 for Retinal Pigment Epithelium (RPE) and Rhodopsin for Rod and L/M Opsin for Cone photoreceptors, respectively. Wide-field color fundus imaging and confocal scanning laser ophthalmoscopy (cSLO) imaging was also taken as in-life readouts for the GFP signal. Eyes from uninjected animals were used as a negative control for GFP immunohistochemistry. The eye sections were assessed for colocalization of GFP with the cell type-specific markers and histopathological examination. Detailed data of GFP staining in ocular structures are presented as a function of time.

ARRDC1-GFP
(SEQ ID NO: 50)
atggggcgagtgcagctcttcgagatcagcctgagccacggccgcgtcgtctacagccccggggagccgttggctgggaccgtgc
gcgtgcgcctgggggcaccgctgccgttccgagccatccgggtgacctgcataggttcctgcggggtctccaacaaggctaatgaca
cagcgtgggtagtggaggagggttacttcaacagttccctgtcgctggcagacaaggggagcctgcccgctggagagcacagcttc
cccttccagttcctgcttcctgccactgcacccacgtcctttgagggtcctttcgggaagatcgtgcaccaggtgagggccgccatcca
cacgccacggttttccaaggatcacaagtgcagcctcgtgttctatatcttgagccccttgaacctgaacagcatcccagacattgagca
acccaacgtggcctctgccaccaagaagttctcctacaagctggtgaagacgggcagcgtggtcctcacagccagcactgatctccg
cggctatgtggtggggcaggcactgcagctgcatgccgacgttgagaaccagtcaggcaaggacaccagccctgtggtggccagtc
tgctgcagaaagtgtcctataaggccaagcgctggatccacgacgtacggaccattgcggaggtggagggtgcgggcgtcaaggc
ctggcggcgggcgcagtggcacgagcagatcctggtgcctgccttgccccagtcggccctgccgggctgcagcctcatccacateg
actactacttacaggtctctctgaaggcgccggaagctactgtgaccctcccggtcttcattggcaatattgctgtgaaccatgccccagt
gagcccccggccaggcctggggctgcctcctggggccccacccctggtggtgccttccgcaccaccccaggaggaggctgaggct
gaggctgcggctggcggcccccacttcttggaccccgtcttcctctccaccaagagccattcgcageggcagcccctgctggccacc
ttgagttctgtgcctggtgcgccggagccctgccctcaggatggcagccctgcctcacacccgctgcaccctcccttgtgcatttcaac
aggtgccactgtcccctactttgcagagggctccggggggccagtgcccactaccagcaccttgattcttcctccagagtacagttcttg
gggctacccctatgaggccccaccgtcttatgagcagagctgcggcggcgtggaacccagcctgacccctgagagcggcggcagc
agcggcatggtgagcaagggcgaggagctgttcaccggggtggtgcccatcctggtcgagctggacggcgacgtaaacggccac
aagttcagcgtgtccggcgagggcgagggcgatgccacctacggcaagctgaccctgaagttcatctgcaccaccggcaagctgcc
cgtgccctggcccaccctcgtgaccaccctgacctacggcgtgcagtgcttcagccgctaccccgaccacatgaagcagcacgactt
cttcaagtccgccatgcccgaaggctacgtccaggagcgcaccatcttcttcaaggacgacggcaactacaagacccgcgccgagg
tgaagttcgagggcgacaccctggtgaaccgcatcgagctgaagggcatcgacttcaaggaggacggcaacatcctggggcacaa
gctggagtacaactacaacagccacaacgtctatatcatggccgacaagcagaagaacggcatcaaggtgaacttcaagatccgcca
caacatcgaggacggcagcgtgcagctcgccgaccactaccagcagaacacccccatcggcgacggccccgtgctgclgcccga
caaccactacctgagcacccagtccgccctgagcaaagaccccaacgagaagcgcgatcacatggtcctgctggagttcgtgaccg
ccgccgggatcactctcggcatggacgagctgtacaagtaa
ARRDC1-GFP
(SEQ ID NO: 51)
MGRVOLFEISLSHGRVVYSPGEPLAGTVRVRLGAPLPFRAIRVTCIGSCGVSNKANDTAWVV
EEGYFNSSLSLADKGSLPAGEHSFPFQFLLPATAPTSFEGPFGKIVHQVRAAIHTPRFSKDH
KCSLVFYILSPLNLNSIPDIEQPNVASATKKFSYKLVKTGSVVLTASTDLRGYVVGQALQLH
ADVENQSGKDTSPVVASLLQKVSYKAKRWIHDVRTIAEVEGAGVKAWRRAQWHEQILVPALP
QSALPGCSLIHIDYYLQVSLKAPEATVTLPVFIGNIAVNHAPVSPRPGLGLPPGAPPLVVPS
APPQEEAEAEAAAGGPHFLDPVFLSTKSHSQRQPLLATLSSVPGAPEPCPQDGSPASHPLHP
PLCISTGATVPYFAEGSGGPVPTTSTLILPPEYSSWGYPYEAPPSYEQSCGGVEPSLTPESG

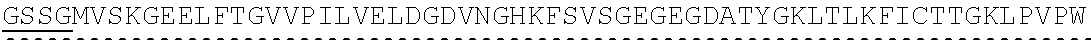

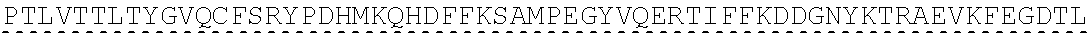

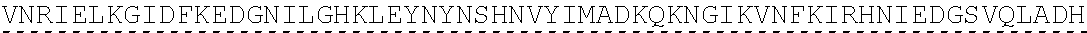

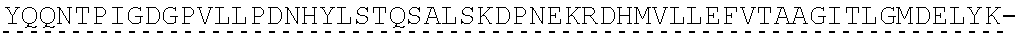

(ARRDC1: underlined; Linker: double underlined; GFP: dashed
underline).

### Surgical Dosing Procedures

Test animals were anesthetized and placed in lateral recumbency. Topical Proparacaine was applied to the eye. The conjunctival fornices were flushed with a 1:50 dilution of betadine solution/saline and the eyelid margins swabbed with undiluted 5% betadine solution. One eve was draped and a wire eyelid speculum is placed. A lateral canthotomy was performed using Stevens tenotomy scissors. A caliper was used to mark spots approximately 3 mm posterior to the limbus on the superotemporal and inferotemporal sclera. Bipolar cautery was used to cauterize the sclera under the marked spots, followed by topical application of undiluted 5% betadine solution. Scleral fixation forceps were used to fix the globe position while a microvitreoretinal blade with a valved cannula being inserted at each marked spot, pushed through the conjunctiva and sclera, and advanced into the vitreous humor. The trocar was positioned to face the posterior axis of the globe, and retracted to leave the scleral port in place. A 31 g needle was inserted tangentially through the limbus and into the anterior chamber to remove 100 μL aqueous humor. A direct contact surgical lens was placed on the cornea with sterile coupling gel. An endoilluminator probe was inserted through one of the scleral ports to facilitate direct visualization of the posterior segment through the microscope. A subretinal injection cannula was inserted through the second port and advanced into the mid-vitreous. The small diameter injection cannula was then advanced until it contacted the retinal surface. The ARMMs particles (i.e., ARRDC1-GFP) are then slowly delivered to induce and fill subretinal blebs. Upon appropriate bleb formation being visualized, the injection was continued to deliver the entire dose volume (e.g., 50 μL/bleb) into the subretinal space. If bleb formation was not visualized, the small diameter injection cannula was repositioned and the injection was attempted again at the same location or an alternative location depending on the surgeon's preference. Subretinal images were taken were taken to capture the location of blebs.

Once the injection dose(s) were delivered, the injection cannula and endoilluminator probe were removed from the scleral ports, and the contact lens was removed from the cornea. The scleral ports were then removed. The lateral canthotomy site was closed using a 7-0 Vicryl suture. Any timed functions are based on the last injection of the last eye dosed. An identical injection procedure was performed on the contralateral eve.

In one embodiment illustrating the techniques in this Example, four female and four male naïve Göttingen minipig test animals (Marshall Bioresources, North Rose, New York), at least four months of age at the time of study, were divided equally into four group of two were dosed subrationally with ARRDC1-GFP-ARMMS according to Table 1 below.

TABLE 1
Subretinal Injection of ARRDC1-GFP-ARMMs into Göttingen Minipigs

| | | Dose | Dose | Dose | | No. of | |
|---|---|---|---|---|---|---|---|
| Group | Treatment | Level | Volume | Concentration | No. of | Animals | |
| No. | Both Eyes | (EVs/eye) | (μL/eye) | (EVs/mL) | Animals | Male | Female |
| 1 | Control | 0 | 0 | 0 | 2 | 1 | 1 |
| | (Untreated) | | | | | | |
| 2 | ARRDC1-GFP | 1.5E12 | 150 μL | 1E13 | 2 | 1 | 1 |
| | | | (3 × 50 μL) | | | | |
| 3 | ARRDC1-GFP | 1.5E12 | 150 μL | 1E13 | 2 | 1 | 1 |
| | | | (3 × 50 μL) | | | | |
| 4 | ARRDC1-GFP | 1.5E12 | 150 μL | 1E13 | 2 | 1 | 1 |
| | | | (3 × 50 μL) | | | | |

Pre-operative in-life procedures were performed in accordance with standard operating procedures for the test facility. Test animals were housed, fed, watered, identified, and handled according to standard USDA protocols and methods before and during the study.

### Postoperative Procedures

Post-operative procedures were conducted in accordance with standard operating procedures for the test facility. Incision site checks and pain scoring were not required following this minimally invasive procedure. Ophthalmology endpoints were interpreted by an ophthalmologist.

The ophthalmoscopic examinations were conducted by an ophthalmologist via indirect ophthalmoscopy and slit-lamp biomicroscopy. Uveitis Scoring was performed, at the time of eye examinations. Wide-field, color fundus imaging was conducted on anesthetized animals using the Clarity RetCam Shuttle (MediMark, Grenoble, France). Confocal scanning laser ophthalmoscopy (cSLO) imaging with green fluorescent protein (GFP) imaging was conducted on anesthetized animals using the Spectralis HRA/OCT System (Heidelberg Engineering Inc., Franklin, MA).

### Terminal Procedures

Euthanasia will be by euthanasia solution administration, under sedation, if necessary, followed by a facility approved final procedure if required. Terminal procedures are summarized in Table 2 below.

TABLE 2
Terminal Procedures

| | | Necropsy | | |
|---|---|---|---|---|
| | Scheduled | Procedures | | |
| | Euthanasia | Tissue | Histology | Microscopic |
| Group No. | Day | Collection | Processing | Evaluation |
| 1 | Day 1 | Eyes Only | Eyes Only | Eyes Only |
| 2 | 6 hours | Eyes Only | Eves Only | Eyes Only |
| | Post Dose | | | |
| 3 | 12 hours | Eyes Only | Eyes Only | Eyes Only |
| | Post Dose | | | |
| 4 | 24 hours | Eyes Only | Eyes Only | Eyes Only |
| | Post Dose | | | |

“Histology Processing” comprised embedding in paraffin, sectioning, mounting on glass slides, and staining with hematoxylin and eosin.

Samples from control eyes (i.e., Group 1) were collected separately from those administered test article. Eyes from all animals were collected for ocular histology. Following termination, eyes were permanently marked for orientation at the 12 o'clock position. Eyes with proximal optic nerves were enucleated. After enucleation, orientation was remarked. The right and left eyes were labelled separately and stored in Davidson's fixative for 24-48 hours and subsequently stored in 70% ethanol for up to an additional 72 hours.

Ocular histology was conducted on trim eves with a single cut sagittal to the optic nerve. Standardly the left eye was trimmed with a single cut sagittal at the optic nerve at a plane that includes the optic disc. Standardly the right eye was trimmed with a single cut superior to the optic nerve at a plane that includes both optic disc. The halved globe containing the injection blebs were submitted with the cut side down in the cassette. The lens and a transverse section of the optic nerve were submitted in a separate block. The eyes were step sectioned as necessary to include sections through the subretinal injection blebs. Sections from each block were collected as follows: Level 1 (rough into the eye until the optic disc is exposed and captured (Booler H S, et al., “Scientific and Regulatory Policy Committee Points to Consider: Fixation, Trimming, and Sectioning of Nonrodent Eyes and Ocular Tissues for Examination in Ocular and General Toxicity Studies. Toxicol Pathol., 50(2):235-251 [2022])): Level 2 (sections taken approximately 500 microns past Level 1): Level 3 (sections taken approximately 500 microns past Level 2); Level 4 (sections taken approximately 500 microns past Level 3); Level 5 (sections taken approximately 500 microns past Level 4); Level 6 (rough the second block to obtain a full face of the tissue, collected after full exposure); Level 7 (sections taken approximately 500 microns past Level 6); Level 8 (sections taken approximately 500 microns past Level 7); Level 9 (sections taken approximately 500 microns past Level 8); and Level 10 (sections taken approximately 500 microns past Level 9). Each level consists of one hematoxylin and eosin stained and eight unstained slides. The block only containing the lens and optic nerve has only hematoxylin and eosin collected initially.

Collected tissues were then evaluated histopathologically by light microscopy using stains and other molecules to highlight moieties on cells and tissues of interest (e.g., RPE65 (retinal pigmented epithelium cells), L/M opsin (cone outer segments), Rhodopsin (rod outer segments), and the like) using standard procedures and protocols.

Additionally, tissues were selected for immunohistochemical analysis. These tissues were preserved in Davidson's fixative, or other designated fixatives for 24-48 hours. Tissues may also be held in 70% ethanol for up to an additional 72 hours prior to processing to paraffin.

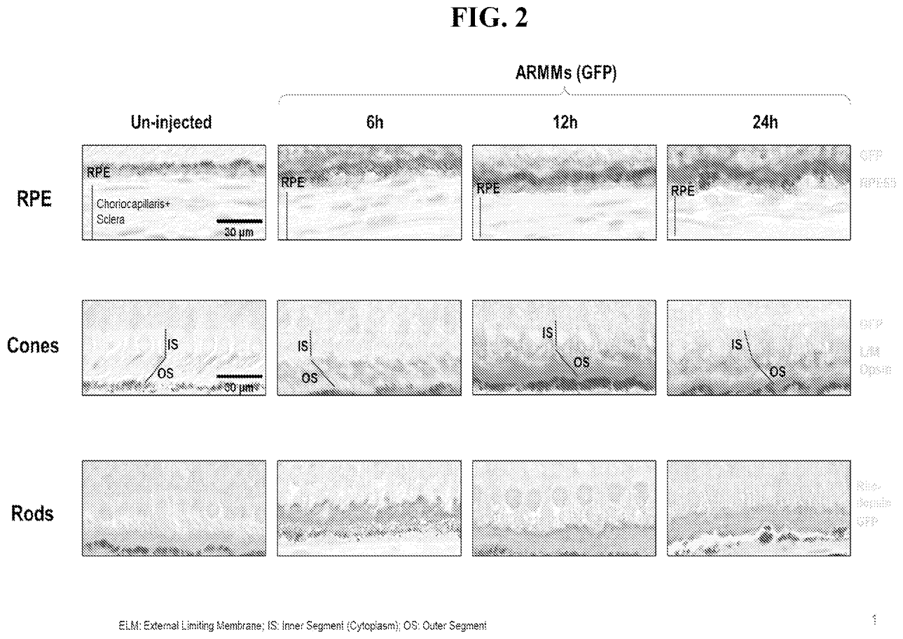

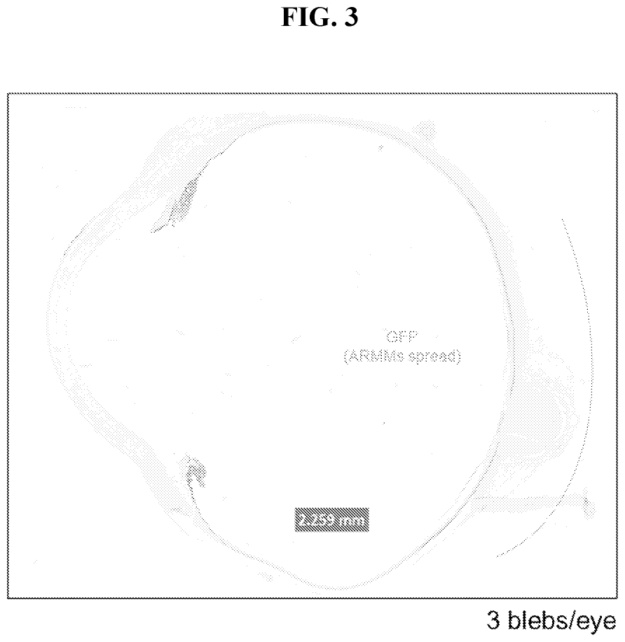

The results from this Example are shown in FIG. 2 and FIG. 3.

### Example 3

### Evaluation in Subretinal Injection of ARMMs into Nonhuman Primates

In this example, nonhuman primates, specifically. St. Kitt's African Green monkeys (Chlorocebus sabaeus) are selected using baseline health assessment techniques including, complete blood count (CBC), general well-being, and ocular health by tonometry, slit lamp biomicroscopy, fundoscopy, color and fluorescence fundus imaging for subsequent use in subretinal injection administration study with ARRDC1-mCherry-ARMMs (SEQ ID NO: 52 and SEQ ID NO: 53). Baseline screening and all subsequent procedures were performed under sedation with intramuscular ketamine (8 mg/kg) and xylazine (1.6 mg/kg) to effect, and pupil dilation with topical 10% phenylephrine, 1% tropicamide and/or 1% cyclopentolate. Animals with normal findings were enrolled in the administration study and assigned to treatment groups as described in Table 3.

TABLE 3
Nonhuman Primate Groups

| | | | | | | | Dose | No. of | |
|---|---|---|---|---|---|---|---|---|---|
| | | | | | Concentration | Volume | (vesicles/ | Animals | |
| Group | N | Eye | Treatment | Route | (vesicles/mL) | (μL) | eye) | Male | Female |
| 1 | 1 | OD | no treatment | — | — | — | — | 1 Female | |
| | | OS | PBS | SR | — | 100 | — | | |
| 2 | 2 | OU | ARRDC1-mCherry | SR | 1 × 10¹³ | 100 | 1 × 10¹² | 1 | 1 |
| 3 | 2 | OU | ARRDC1-mCherry | SR | 1 × 10¹³ | 100 | 1 × 10¹² | 1 | 1 |

ARRDC1-mCherry
(SEQ ID NO: 52)
atggggcgagtgcagctcttcgagatcagcctgagccacggccgcgtcgtctacagccccgg
ggagccgttggctgggaccgtgcgcgtgcgcctgggggcaccgctgccgttccgagccatcc
gggtgacctgcataggttcctgcggggtctccaacaaggctaatgacacagcgtgggtagtg
gaggagggttacttcaacagttccctgtcgctggcagacaaggggagcctgcccgctggaga
gcacagcttccccttccagttcctgcttcctgccactgcacccacgtcctttgagggtcctt
tcgggaagatcgtgcaccaggtgagggccgccatccacacgccacggttttccaaggatcac
aagtgcagcctcgtgttctatatcttgagccccttgaacctgaacagcatcccagacattga
gcaacccaacgtggcctctgccaccaagaagttctcctacaagctggtgaagacgggcagcg
tggtcctcacagccagcactgatctccgcggctatgtggtggggcaggcactgcagctgcat
gccgacgttgagaaccagtcaggcaaggacaccagccctgtggtggccagtctgctgcagaa
agtgtcctataaggccaagcgctggatccacgacgtacggaccattgcggaggtggagggtg
cgggcgtcaaggcctggcggcgggcgcagtggcacgagcagatcctggtgcctgccttgccc
cagtcggccctgccgggctgcagcctcatccacatcgactactacttacaggtctctctgaa
ggcgccggaagctactgtgaccctcccggtcttcattggcaatattgctgtgaaccatgccc
cagtgagcccccggccaggcctggggctgcctcctggggccccacccctggtggtgccttcc
gcaccaccccaggaggaggctgaggctgaggctgcggctggcggcccccacttcttggaccc
cgtcttcctctccaccaagagccattcgcagcggcagcccctgctggccaccttgagttctg
tgcctggtgcgccggagccctgccctcaggatggcagccctgcctcacacccgctgcaccct
cccttgtgcatttcaacaggtgccactgtcccctactttgcagagggctccggggggccagt
gcccactaccagcaccttgattcttcctccagagtacagttcttggggctacccctatgagg
ccccaccgtcttatgagcagagctgcggcggcgtggaacccagcctgacccctgagagcggc
ggcagcagcggcatggtgagcaagggcgaggaggataacatggccatcatcaaggagttcat
gcgcttcaaggtgcacatggagggctccgtgaacggccacgagttcgagatcgagggcgagg
gcgagggccgcccctacgagggcacccagaccgccaagctgaaggtgaccaagggtggcccc
ctgcccttcgcctgggacatcctgtcccctcagttcatgtacggctccaaggcctacgtgaa
gcaccccgccgacatccccgactacttgaagctgtccttccccgagggcttcaagtgggagc
gcgtgatgaacttcgaggacggcggcgtggtgaccgtgacccaggactcctccctgcaggac
ggcgagttcatctacaaggtgaagctgcgcggcaccaacttcccctccgacggccccgtaat
gcagaagaagaccatgggctgggaggcctcctccgagcggatgtaccccgaggacggcgccc
tgaagggcgagatcaagcagaggctgaagctgaaggacggcggccactacgacgctgaggtc
aagaccacctacaaggccaagaagcccgtgcagctgcccggcgcctacaacgtcaacatcaa
gttggacatcacctcccacaacgaggactacaccatcgtggaacagtacgaacgcgccgagg
gccgccactccaccggcggcatggacgagctgtacaagtag
(SEQ ID NO: 53)
MGRVQLFEISLSHGRVVYSPGEPLAGTVRVRLGAPLPFRAIRVTCIGSCGVSNKANDTAWVV
EEGYFNSSLSLADKGSLPAGEHSFPFQFLLPATAPTSFEGPFGKIVHQVRAAIHTPRESKDH
KCSLVFYILSPLNLNSIPDIEQPNVASATKKESYKLVKTGSVVLTASTDLRGYVVGQALQLH
ADVENQSGKDTSPVVASLLQKVSYKAKRWIHDVRTIAEVEGAGVKAWRRAQWHEQILVPALP
QSALPGCSLIHIDYYLQVSLKAPEATVTLPVFIGNIAVNHAPVSPRPGLGLPPGAPPLVVPS
APPQEEAEAEAAAGGPHELDPVFLSTKSHSQROPLLATLSSVPGAPEPCPODGSPASHPLHP
PLCISTGATVPYFAEGSGGPVPTTSTLILPPEYSSWGYPYEAPPSYEQSCGGVEPSLTPESG

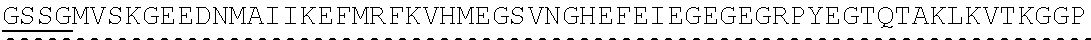

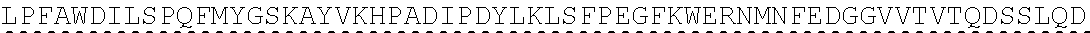

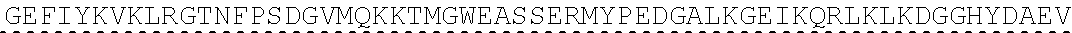

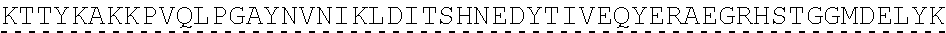

(ARRDC1: underlined; Linker: double underlined; mCherry: dashed
underline)

### Surgical Dosing Procedures

Topical 1% atropine gel was administered to each eye one day prior to subretinal dosing. This procedure was followed with topical 10% phenylephrine, 1% tropicamide and/or 1% cyclopentolate on the day of dosing in eyes not fully dilated. Test animals received one subretinal injection in both eyes oculus uterque (OU) in accordance with the treatment assignments in Table 3. After eye speculum placement, a drop of proparacaine hydrochloride 0.5% was administered. After 30 seconds, a 5% Betadine® solution was administered, followed by a sterile saline rinse. A sterile eye drape and lid speculum was then placed on the respective animals. A 25-gauge vitrectomy port, Alcon© Surgery Valved Entry System 1-CT (Alcon Surgery Inc., Fort Worth, TX) was placed via port introducer device at the level of the Ora serrata in the superotemporal quadrant. A second vitrectomy port was placed at the level of the Ora serrata in the inferotemporal quadrant. After vitrectomy port placement, a contact vitrectomy lens was placed and centered on the cornea, employing 0.9% saline as a coupling agent. With the surgeon positioned temporally, a 25-gauge light pipe was inserted through the vitrectomy port on the left into the vitreous cavity for intraocular illumination, keeping the tip in the anterior vitreous. A subretinal cannula, MedOne Surgical 25/38 g, part number 3237. (MedOne Surgical Inc., Sarasota, FL) attached to a MedOne microfluid injector, part number 3275, coupled to an Alcon Constellation Vision System was introduced through the second vitrectomy port. The 38-gauge flexible microtip was advanced to gently touch the retinal surface, targeting a point in the superotemporal region within approximately 2 disc diameters superior to the fovea. Upon observing slight blanching of the retinal surface at the point of contact, test article was injected at a pressure sufficient to elevate the bleb then adjusted to maintain within 4-6 mmHg. Pressure threshold was set at 14 mmHg. When an initial bleb raised, a target volume (Table 1) of test article was administered. The surgical instruments were removed following injection and the sclerotomies self-sealed. A topical antibiotic ointment was instilled in the eye after post-operative fundus imaging to document subretinal bleb location and dimension.

### Postoperative Procedures

Intraocular pressure (IOP) was measured OU using a Tonovet Plus tonometer (iCare Inc., Raleigh, NC) set to the cat (c) calibration setting. Three measures were taken from each eye and the mean IOP calculated.

Eyes oculus uterque were examined by slit lamp biomicroscopy and retinoscopy using a 90-diopter lens. Scoring was applied to qualitative clinical ophthalmic findings using a nonhuman primate ophthalmic scoring system and summary score derived from exam components.

Color anterior and fundus imaging and fluorescent fundus imaging was used to detect GFP expression performed OU with a 500 field of view centered on the macula with additional peripheral images acquired in each quadrant using a Topcon TRC-50EX (Topcon Medical Systems, Inc., Paramus. NJ) retinal camera with Canon 6D digital imaging hardware and New Vision Fundus Image Analysis System software (URAL Telekom, Antalya, Turkey). Color fundus photos were captured with shutter speed (Tv) 1/25 sec, ISO 400 and flash 18. Monochromatic and color fluorescent images were captured with exciter and barrier filters engaged (587 nm exciter/610 nm barrier filter), Tv 1/5 sec, ISO 3200 and flash 300. Fluorescence photographs were evaluated using a scoring system to define extent of mCherry expression with quantitative analysis applied as appropriate where a score of 0=absent; 1=trace; 2=slight; 3=moderate; 4=bright and 5=intense in the areas where bleb formation is seen post-dosing, foveal, peripheral, and perivascular regions of the eye.

The head, torso, limbs, and integument of each test animal were evaluated, auscultation performed, and vitals assessed, including temperature, and heart and respiratory rate measured manually over a 15 second interval.

0.5 mL blood, was transferred to K2EDTA lavender top vacutainers, gently inverted several times and retained on ice for complete blood count with differential analysis on an Abaxis VetScan HM5 hematology system (Abaxis. Inc., Union City. CA).

### Terminal Procedures

Test animals were euthanized at study terminus with ketamine (8 mg/kg IM) and xylazine (1.6 mg/kg IM) followed by sodium pentobarbital (100 mg/kg) administered intravascularly over a period of ˜10 seconds to effect. After loss of corneal reflex, and prior to sample collection, animals were exsanguinated by incising the caudal vena cava followed by immediate perforation of the diaphragm relieving negative intrathoracic pressure.

Post-mortem veterinary examination of organs including external features of the carcass, external body orifices, abdominal, thoracic, and cranial cavities, organs, and tissues were performed to identify and document any gross abnormality or pathology.

After placing a suture in the limbus at the 12 o'clock position both globes oculus uterque were enucleated, excess orbital tissue trimmed, immersion fixed in Davidson's for 24 hours before being transferred to phosphate buffered saline with 0.05% sodium azide.

Fixed globes were embedded in paraffin and sectioned in twenty stepped horizontal sections spanning peripheral and macular retinal regions and stained with hematoxylin and eosin for analysis by a board-certified veterinary pathologist. Globes were trimmed in a sagittal fashion in three sections prior to processing, with the mid-section encompassing the pupil-optic nerve.

Ocular specimens were sectioned with a microtome as follows: nasal and temporal calottes, and optic nerve three levels with a minimum of 80 μm between levels. One slide was collected at each level. The pupil-optic nerve was sectioned in five levels with a minimum of 80 μm in between each level, spanning macular and peripheral retinal regions with ten slides collected at each level. One slide from each level will be stained with hematoxylin and eosin.

A fit for purpose, monoplex, immunofluorescence assay for mCherry was optimized for specificity and sensitivity with clear signal:noise differentiation using existing tissue samples. One slide from each eye pupil-optic nerve section was selected for mCherry/cell marker duplex immunofluorescence. All stains and assays were performed according to routine laboratory methods and microscopically quality checked.

Digital whole slide images of all stained glass microscope slides were generated using a 3DHistech P150 digital slide scanner (3DHISTECH Ltd, Budapest, Hungary). Slides were scanned using the 20× objective (49× native magnification) and quality checked to ensure complete image capture and lack of significant scanning artifacts.

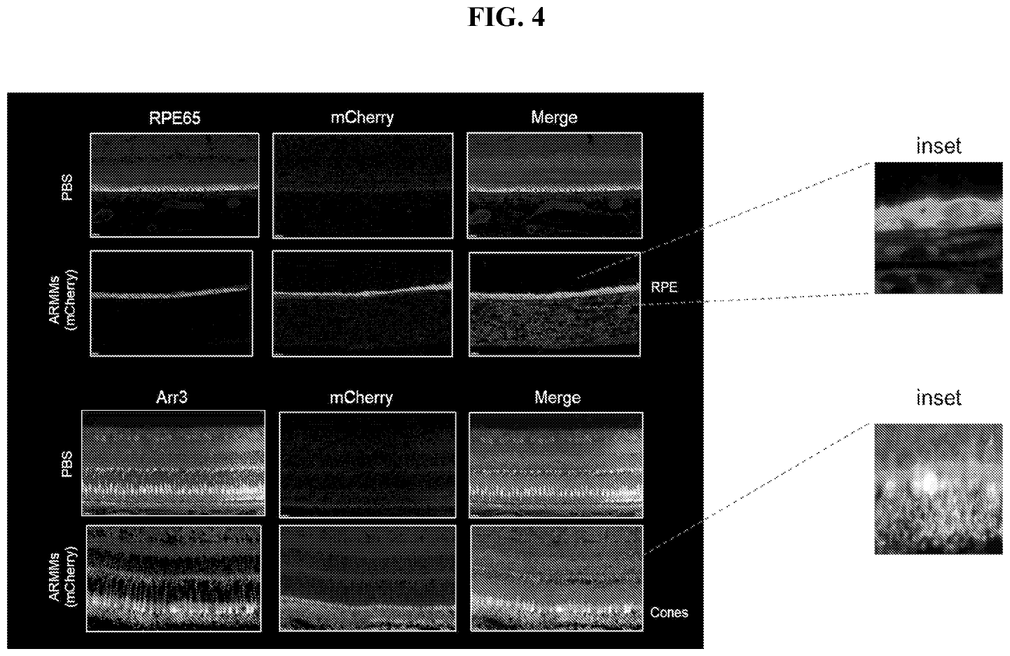

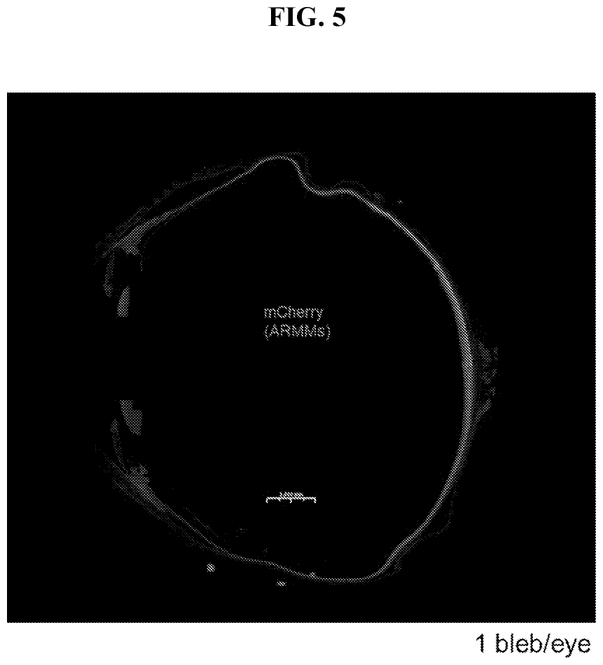

The results from this Example are shown in FIG. 4 and FIG. 5.

### REFERENCES

All publications, patents and sequence database entries mentioned herein, including those items listed above, are hereby incorporated by reference in their entirety as if each individual publication or patent was specifically and individually indicated to be incorporated by reference. In case of conflict, the present application, including any definitions herein, will control.

### EQUIVALENTS AND SCOPE

Those skilled in the art will recognize or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all the group members are present in, employed in, or otherwise relevant to a given product or process.

Additionally, although the terms “first,” “second,” etc., may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus, for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all the embodiments in which one or more elements, features, purposes, or aspects is excluded are not set forth explicitly herein.

What is claimed is:

1. An arrestin domain-containing protein 1 (ARRDC1)-mediated microvesicle (ARMM), comprising:
(i) a lipid bilayer and an ARRDC1 protein,
(ii) a molecule, and
(iii) a viral envelope protein.

2. The microvesicle of claim 1, wherein said viral envelope protein is vesicular stomatitis virus G (VSV-G).

3. The microvesicle of claim 1, wherein said viral envelope protein is rabies virus glycoprotein (RVG).

4. A microvesicle-producing cell comprising:
a recombinant expression construct encoding an ARRDC1 protein or a variant thereof under the control of a heterologous promoter,
and a viral envelope protein.

5. The microvesicle-producing cell of claim 4, wherein said viral envelope protein is VSV-G.

6. The microvesicle-producing cell of claim 4, wherein said viral envelope protein is RVG.

7. A method of delivering a molecule to a target cell, said method comprising contacting the target cell with the microvesicle of any of claims 1-3.

8. The method of claim 7, wherein said target cell is a cell of the eye.

9. The method of claim 8, wherein said cell of the eye is a cell of the retina.

10. A method of treating a disorder in a patient, the method consisting of administering to the patient a microvesicle of any of claims 1-3.

11. A method of treating a disorder in a patient, the method consisting of administering to the patient a microvesicle-producing cell of any of claims 4-6.

12. The method of claim 10 or 11, wherein said disorder is a disorder of the eve.

13. An arrestin domain-containing protein 1 (ARRDC1)-mediated microvesicle (ARMM), comprising:
(i) a lipid bilayer and an ARRDC1 protein, and
(ii) a therapeutic agent.

14. The method of claim 10 or 11, wherein said patient is a mammal.

15. The method of claim 14, wherein said mammal is a primate.

16. The method of claim 15, wherein said primate is a human.

17. An arrestin domain-containing protein 1 (ARRDC1)-mediated microvesicle (ARMM) for use a medicament.

18. An arrestin domain-containing protein 1 (ARRDC1)-mediated microvesicle (ARMM) for use in the treatment of diseases of the eye.

19. The use of said arrestin domain-containing protein 1 (ARRDC1)-mediated microvesicle (ARMM) of claim 18, wherein said diseases are Stargardt Macular Dystrophy or Age-Related Macular Degeneration.

20. A composition substantially as shown and described.

21. A method substantially as shown and described.
